(19) United States
(12) Patent Application Publication
(10) Pub. No.: US 2025/0262248 A1
(43) Pub. Date: Aug. 21, 2025

(54) METHODS AND COMPOSITIONS FOR TREATING VISION LOSS

(71) Applicants: CELL CURE NEUROSCIENCES, LTD., Jerusalem (IL); HADASIT MEDICAL RESEARCH SERVICS AND DEVELOPMENT LTD., Jerusalem (IL)

(72) Inventors: Rami SKALITER, Ness Ziona (IL); Dana HAYOUN NEEMAN, jerusalem (IL); Ofer WISER, Jerusalem (IL); Benjamin Eithan REUBINOFF, Jerusalem (IL); Hanita KHANER, Jerusalem (IL)

(21) Appl. No.: 18/859,306

(22) PCT Filed: Apr. 24, 2023
(86) PCT No.: PCT/US2023/019657

§ 371 Date: Oct. 23, 2024

Related U.S. Application Data
(60) Provisional application No. 63/334,593, filed on Apr. 25, 2022.

(51) Int. Cl. A61K 35/30; A61K 9/00; C12N 5/0735; C12N 5/074; C12N 5/079

(52) U.S. Cl. CPC A61K 35/30; A61K 9/0048; C12N 5/0606; C12N 5/0621; C12N 5/0696; C12N 2500/33; C12N 2500/38; C12N 2501/105; C12N 2506/02; C12N 2506/45

(57) ABSTRACT

Described herein are methods for differentiating human pluripotent stem cells into populations of retinal cells comprising photoreceptor neural cells (PNCs) in dynamic culture. Also provided are cells and cellular compositions obtained by such methods, and uses of such cells in a suspension or imbedded in a scaffold that can be administered to a subject suffering from a retinal disease or disorder.

### RELATED APPLICATIONS

This application claims priority to, and the benefit of, U.S. Provisional Application No. 63/334,593, filed on Apr. 25, 2022. The contents of the aforementioned patent application are incorporated herein by reference in their entirety.

### BACKGROUND

Globally, at least 2.2 billion people have a visual impairment. In the United States alone, cases of early age-related macular degeneration are expected to double by 2050, from 9.1 million to 17.8 million for those aged 50 years or older and cases of diabetic retinopathy among people aged 65 or older are expected to quadruple by 2050, from 2.5 million to 9.9 million (www.cdc.gov/visionhealth/pdf/vision_brief.pdf). The leading causes of blindness and low vision in the United States are primarily age-related eye diseases such as age-related macular degeneration and diabetic retinopathy, however genetic disorders also play a role. In many cases, the cause of the vision loss is related to the degeneration and death of cells in the retina and associated structures.

Retinal cells, such as photoreceptor neural cells (PNCs), are intended as cellular therapy for neurodegenerative patients that suffer from significant loss of visual acuity. The implanted retinal cells should present high survivability and connectivity to surrounding functional layers, including functional neural connectivity, for the restoration of vision. The cellular therapy using retinal cells may be used for a partly or fully blind patient with extensive damage, which may include irrecoverable damage to the photoreceptor cells, as well as patients of neurodegenerative diseases with direct retinal damage to the photoreceptor cells, such as retinitis pigmentosa or Stargardt's disease, both of which are genetic disorders that cause significant damage at a relatively early age. As such, improved methods and compositions comprising populations of retinal cells are needed.

### SUMMARY

Provided herein are, inter alia, methods for differentiating human pluripotent stem cells to populations of cells comprising retinal cells, such as photoreceptor neural cells (PNCs), in a dynamic culture, which may be adapted to a commercial scale process. Also provided herein are compositions comprising cells and cell populations obtained by the methods described herein. Also provided herein are methods for treating retinal diseases, including for example vision loss, using the cells and cell populations obtained by the methods described herein.

The disclosure provides a pharmaceutical composition comprising a population of retinal cells, wherein (a) greater than or equal to 10% of the cells in the population express Cone-rod homeobox (Crx); (b) greater than or equal to 3% of the cells in the population express Recoverin; (c) greater than or equal to 3% of the cells in the population express Cone arrestin (CAR); and (d) less than or equal to 1% of the cells in the population express TRA-1-60 and/or SSEA5; wherein the pharmaceutical composition comprises a pharmaceutically acceptable carrier.

In some embodiments of the pharmaceutical compositions of the disclosure, greater than or equal to 15% of the cells in the population express Crx. In some embodiments, greater than or equal to 5% of the cells in the population express Recoverin. In some embodiments, greater than or equal to 6% of the cells in the population express CAR.

In some embodiments, the population of retinal cells comprises cells that express at least one marker selected from the group consisting of SIX homeobox 3 (six3), SIX homeobox 6 (six6), phosphodiesterase 6H (PDE 6H), visual system homeobox 2 (CHX10 or VSX2), premelanosome protein (PMEL), protein kinase C alpha (PKCa), ELAV like RNA binding protein 3/4 (HuC/D), orthodenticle homeobox 2 (Otx2), neuronal differentiation 1 (NeuroD), B lymphocyte-induced maturation protein-1 (Blimp1), Transducin, Phosducin (PdC), retinoid X receptor gamma (RXRy), thyroid hormone receptor isoform (Tr-32), atonal bHLH transcription factor 7 (Atoh7), insulin gene enhancer protein (Isl-1), retinal homeobox gene 1 (Rx1), paired box 6 (Pax6), LIM homeobox 2 (LHX2), and retina and anterior neural fold homeobox (RAX).

In some embodiments of the pharmaceutical compositions of the disclosure, (e) greater than or equal to 30% of the cells in the population express Pax6; (f) less than or equal to 40% of the cells in the population express beta tubulin 3; (g) less than or equal to 30% of the cells in the population express PMEL; (h) less than or equal to 30% of the cells in the population express PKCa; or (i) less than or equal to 10% of the cells in the population express HuCD. In some embodiments, less than or equal to 0.1% of the cells in the population express TRA-1-60 and/or SSEA5.

In some embodiments of the pharmaceutical compositions of the disclosure, (a) between about 10% to 70% of the cells in the population express Crx; (b) between about 3% to 90% of the cells in the population express Recoverin; (c) between about 3% to 90% of the cells in the population express CAR; and (d) between 0 to about 0.1% of the cells in the population express TRA-1-60 and/or SSEA5.

In some embodiments of the pharmaceutical compositions of the disclosure, the population of retinal cells comprises early eye field cells, embryonic retinal cells, precursors of photoreceptor neuronal cells (PNCs), mature photoreceptor neuronal cells, or any combination thereof. In some embodiments, (i) the early eye field cells comprise cells expressing Six3, Six 6, Rx1, Pax6, RXRy, LHX2 and/or RAX; (ii) the embryonic retinal cells comprise cells express Otx2, NeuroD, Blimp1, Transducin, Phosducin (PdC), RXRy and Tr-32, Atoh7 and/or Isl-1; or (iii) the precursors of PNCS and mature PNCs comprise cells expressing Crx, Recoverin, and/or Cone arrestin (CAR). In some embodiments, the population of retinal cells comprises neuronal retina cells (NRCs), Muller glia cells, retinal pigmented epithelial (RPE) cells, or any combination thereof. In some embodiments, the NRCs comprise PNCs, retinal ganglion cells, horizontal neurons, amacrine neurons, rod bipolar cells, cone bipolar cells, rod photoreceptor cells, cone photoreceptor cells or any combination thereof.

In some embodiments of the pharmaceutical compositions of the disclosure, the pharmaceutical composition comprises a cryopreservation medium. In some embodiments, cryopreservation medium comprises a cryoprotective agent selected from the group consisting of glycerol, sucrose, dextran and dimethyl sulfoxide (DMSO). In some embodiments, the pharmaceutical composition comprises about 0.1% to about 40% of the cryoprotective agent. In some embodiments, the pharmaceutical composition comprises about 1% to about 10% of the cryoprotective agent.

In some embodiments of the pharmaceutical compositions of the disclosure, the population of retinal cells comprises between 5,000 cells and 25 million cells. In some embodiments, the population of retinal cells are at a concentration of 1×10⁵ cells per mL to about 100×10⁶ cells per mL. In some embodiments, the population of cells are in a suspension.

In some embodiments of the pharmaceutical compositions of the disclosure, the population of cells are in a scaffold. In some embodiments, the scaffold is biocompatible and/or biodegradable.

Provided herein are methods for obtaining a population of neural retina cells (NRCs) from undifferentiated pluripotent stem cells, the method comprising: (a) growing the undifferentiated pluripotent stem cells in a static adherent culture or optionally in a dynamic culture; (b) seeding the undifferentiated pluripotent stem cells as single cells in a culture vessel at cell concentration from about 100,000 cell/mL to about 2,000,000 cells/mL; and (c) differentiating the undifferentiated pluripotent stem cells in the culture vessel under conditions to obtain the population of NRCs. In some embodiments, the retinal cells neural retinal cells, for example PNCs, retinal ganglion cells, Horizontal neurons, Amacrine neurons, Rod Bipolar cells, Cone bipolar cells, Rods photoreceptors, and/or Cone photoreceptors, as well as Muller glia cells, retinal pigmented epithelial (RPE) cells. In embodiments, the NRCs comprise PNCs. In embodiments, the retinal cells comprise retinal pigment epithelial cells (RPEs). In embodiments, the retinal cells comprise a mixture of PNCs and RPEs. In embodiments, the retinal cells comprise one or more of PNCs, RPEs, bipolar cells, retinal ganglion cells, horizontal cells, and amacrine cells.

In embodiments, the undifferentiated pluripotent stem cells are human embryonic stem cells (hESCs). In other embodiments, the undifferentiated pluripotent stem cells are human induced pluripotent stem cells (hiPSCs).

In some embodiments, the undifferentiated pluripotent stem cells are seeded for differentiation as single cells and grown in suspension as cell aggregates for at least 14-18 weeks.

In some embodiments, the aggregates have a size controlled by a rotation velocity of the culture vessel. In embodiments, the size of the aggregates is from about 100 μm to about 800 μm. For example, the rotation velocity may be increased during the differentiation process.

In some embodiments, differentiating undifferentiated pluripotent stem cells includes the following:
i) culturing the undifferentiated pluripotent stem cells, for a first time period under culture conditions sufficient to induce the stem cells to form cell aggregates (e.g., organoids), wherein the cell aggregates comprise cells expressing one or more early eye field markers selected from Rx1, Pax6, RXRy, LHX2, Six3, Six6, Otx2 and RAX; and
ii) culturing the cell aggregates from step i) for a second time period under culture conditions sufficient to differentiate the cell aggregates to cells expressing one or more embryonic retina markers selected from Otx2, NeuroD, Blimp 1, Transducin, Phosducin (PdC), RXRy and Tr-32, Atoh7 or Isl-1, and iii) culturing the cells expressing the one or more embryonic retina markers from step ii) for a third time period under culture conditions sufficient to differentiate the cells to precursors of PNCs and mature photoreceptor neural cells, wherein the PNCs express one or more proteins selected from Crx, Recoverin, PDE 6H (phosphodiesterase 6H) and Cone arrestin (CAR). In some embodiments, the first time period is from about 3 days to about 120 days, or from about 5 days to about 15 days, or about 3 days. In embodiments, the second time period is from about 1 days to about 120 days, or from about 5 days to about 15 days, or about 11 days.

In embodiments, differentiating undifferentiated pluripotent stem cells includes the following: (a) differentiating for a first time period with a Rock Inhibitor (RI), nicotinamide (NIC), and a Wnt inhibitor (e.g. IWRe1); (b) differentiating for a second time period with NIC and a Wnt inhibitor (e.g. IWRe1); (c) differentiating for a third time period with NIC and insulin growth factor-1 (IGF-1); (d) differentiating for a fourth time period with NIC, IGF-1 and a Notch inhibitor (e.g. DAPT); and (e) differentiating for a fifth time period with IGF-1, retinoic acid (RA), taurine and NT4. In embodiments, the first time period is 0-7 days, and/or the second time period is 2-20 days, and/or the third time period is 10 days to 8 weeks, and/or the fourth time period is 4-10 weeks, and/or the fifth time period is 12-18 weeks.

The disclosure provides methods of making the pharmaceutical composition of the disclosure, comprising culturing a population of undifferentiated pluripotent stem cells under culture conditions sufficient to (i) differentiate cells in the population of the undifferentiated cells into retinal cells; and (ii) produce a cellular aggregate, wherein the cellular aggregate is between 100 μm-800 μm in diameter.

The disclosure provides methods of making compositions comprising populations of retinal cells, comprising (a) culturing a population of undifferentiated pluripotent stem cells in a first cell culture medium comprising Nicotinamide (NIC), rel-4-[(3aR,4S,7R,7aS)-1,3,3a,4,7,7a-Hexahydro-1,3-dioxo-4,7-methano-2H-isoindol-2-yl]-N-8-quinolinylbenzamide (IWRe), and Rock inhibitor (RI) for a first time period; (b) culturing the population of cells produced in step (a) in a second cell culture medium comprising NIC and IWRe, for a second time period; (c) culturing the population of cells produced in step (b) in a third cell culture medium comprising NIC and Insulin-like growth factor 1 (IGF-1) for a third time period; and (d) collecting the population of cells, thereby producing the composition comprising the population of retinal cells; wherein the populations of cells in are cultured for at least 14 weeks in a culture vessel under conditions sufficient to produce cellular aggregates that are 100 μm to 800 μm in diameter.

In some embodiments of the methods of the disclosure, the cells are cultured in dynamic culture conditions. In some embodiments, the culture vessel is a bioreactor. In some embodiments, the culture vessel comprises a vertical wheel bioreactor, a wave bioreactor, or a Gas Permeable Rapid Expansion bioreactor. In some embodiments, the culture vessel comprises a wheel bioreactor, and the rotational velocity of the wheel bioreactor is increased over time thereby producing cellular aggregates that are 100 μm to 800 μm in diameter that are suspended in the wheel bioreactor. In some embodiments, the rotational velocity of the wheel bioreactor subjects the cellular aggregates to controlled shear stress. In some embodiments, the rotational velocity is between 35 to 80 revolutions per minute (RPM). In some embodiments, the methods comprise an initial rotational velocity of about 30-50 RPM that is increased to about 60-80 RPM by the end of the differentiation process.

In some embodiments of the methods of the disclosure, the first time period is between 1-15 days, the second time period is between 1-30 days, and/or the third time period is between 5 days and 14 weeks.

In some embodiments of the methods of the disclosure, the methods comprise, before step (d), a step comprising (i) culturing the population of cells produced in step (c) in a fourth cell culture medium comprising IGF-1, NIC, and tert-Butyl (S)-{(2S)-2-[2-(3,5-difluorophenyl)acetamido]propanamido}phenylacetate (DAPT) for a fourth time period. In some embodiments, the fourth time period is between 1 week to 18 weeks.

In some embodiments of the methods of the disclosure, the methods comprise, after step (i), a step (ii) comprising culturing the population of cells in step (i) in a fifth cell culture medium comprising IGF-1, Retinoic acid (RA), Taurine (TA), Brain-derived neurotropic factor (BDNF), and Neurorophin-4 (NT4) for a fifth time period. In some embodiments, the fifth time period is 4 weeks to 24 weeks.

In some embodiments, the RI is at a concentration of between about 1-20 μM in the first cell culture medium. In some embodiments, the NIC is at a concentration of between about 1-50 mM in the first, second, third, and/or fourth cell culture medium. In some embodiments, the IWRe is at a concentration of between about 0.01-20 μM in the first and/or second cell culture medium. In some embodiments, the IGF-1 is at a concentration of between about 0.5-20 ng/mL in the third, fourth, and/or fifth cell culture medium. In some embodiments, the DAPT is at a concentration of between about 1-50 μM in the fourth cell culture medium. In some embodiments, the BDNF is at a concentration of between about 5-50 ng/mL in the fifth cell culture medium. In some embodiments, the NT4 is at a concentration of between about 2-200 ng/mL in the fifth cell culture medium. In some embodiments, the TA is at a concentration of between 10-400 μM in the fifth cell culture medium.

The disclosure provides methods of producing a composition comprising a population of retinal cells, the methods comprising: (a) culturing a population of undifferentiated pluripotent stem cells in a first cell culture medium comprising Nicotinamide (NIC) at a concentration of 10 mM, IWRe at a concentration of 3 μm, and Rock inhibitor (RI) at a concentration of 10 μm for at least one day; (b) culturing the population of cells produced in step (a) in a second cell culture medium comprising NIC at a concentration of 10 mM, and IWRe at a concentration of 3 μm for at least 2 days; (c) culturing the population of cells produced in step (b) in a third cell culture medium comprising NIC at a concentration of 10 mM and IGF-1 at a concentration of 5 ng/mL for at least 10 days; and (d) collecting the population of cells, thereby producing the composition comprising the population of retinal cells; wherein the populations of cells in are cultured for at least 14 weeks in a culture vessel under conditions sufficient to produce aggregates that are 100 μm to 800 μm in diameter.

The disclosure provides methods of producing a composition comprising a population of retinal cells, the methods comprising: (a) culturing a population of undifferentiated pluripotent stem cells in a first cell culture medium comprising Nicotinamide (NIC) at a concentration of 10 mM, IWRe at a concentration of 3 μm, and Rock inhibitor (RI) at a concentration of 10 μm for at least one day; (b) culturing the population of cells produced in step (a) in a second cell culture medium comprising NIC at a concentration of 10 mM and IWRe at a concentration of 3 μm, for at least 2 days; (c) culturing the population of cells produced in step (b) in a third cell culture medium comprising NIC at a concentration of 10 mM and IGF-1 at a concentration of 5 ng/mL for at least 10 days; (d) culturing the population of cells produced in step (c) in a fourth cell culture medium comprising IGF-1 at a concentration of 5 ng/mL, NIC at a concentration of 10 mM, and DAPT at a concentration of 10 μM for at least 2 weeks; and (e) collecting the population of cells, thereby producing the composition comprising the population of retinal cells; wherein the populations of cells in are cultured for at least 14 weeks in a culture vessel under conditions sufficient to produce aggregates that are 100 μm to 800 μm in diameter.

The disclosure provides methods of producing a composition comprising a population of retinal cells, the methods comprising (a) culturing a population of undifferentiated pluripotent stem cells in a first cell culture medium comprising Nicotinamide (NIC) at a concentration of 10 mM, IWRe at a concentration of 3 μm, and Rock inhibitor (RI) at a concentration of 10 μm for at last 1 day; (b) culturing the population of cells produced in step (a) in a second cell culture medium comprising NIC at a concentration of 10 mM and IWRe at a concentration of 3 μm for at least 2 days; (c) culturing the population of cells produced in step (b) in a third cell culture medium comprising NIC at a concentration of 10 mM and IGF-1 at a concentration of 5 ng/mL for at least 10 days; (d) culturing the population of cells produced in step (c) in a fourth cell culture medium comprising IGF-1 at a concentration of 5 ng/mL, NIC at a concentration of 10 mM, DAPT at a concentration of 10 μM for at least 2 weeks; (e) culturing the population of cells produced in step (d) in a fifth cell culture medium comprising IGF-1 at a concentration of 5 ng/mL, Retinoic acid (RA) at a concentration of 0.5 μM, Taurine (TA) at a concentration of 100 μM, Brain-derived neurotropic factor (BDNF) at a concentration of 20 ng/mL, and Neurorophin-4 (NT4) at a concentration of 20 ng/mL for at least 5 weeks; and (f) collecting the population of cells, thereby producing the composition comprising the population of retinal cells; wherein the populations of cells in are cultured for at least 14 weeks in a culture vessel under conditions described in claim 52 sufficient to produce aggregates that are 100 μm to 800 μm in diameter.

In some embodiments of the methods of the disclosure, the undifferentiated pluripotent stem cells comprise human embryonic stem cells (hESCs) or human induced pluripotent stem cells (hiPSCs). In some embodiments, the hESCs comprise HADC102 cells.

In some embodiments of the methods of the disclosure, the methods comprise, prior to step (a), seeding the undifferentiated pluripotent stem cells as single cells in a culture vessel at a density of 50,000 cell/mL to about 2,000,000 cells/mL.

In some embodiments of the methods of the disclosure, the population of retinal cells comprises early eye field cells, embryonic retinal cells, precursors of photoreceptor neuronal cells (PNCs), mature photoreceptor neuronal cells, or any combination thereof. In some embodiments, the population of retinal cells comprises neuronal retina cells (NRCs), Muller glia cells, retinal pigmented epithelial (RPE) cells, or any combination thereof. In some embodiments, the NRCs comprise PNCs, retinal ganglion cells, horizontal neurons, amacrine neurons, rod bipolar cells, cone bipolar cells, rod photoreceptor cells, cone photoreceptor cells or any combination thereof.

In some embodiments of the methods of the disclosure, the methods comprise, prior to step (d): (i) collecting the cellular aggregates; (ii) dissociating the cellular aggregates to produce dissociated cells; (iii) seeding the dissociated cells in tissue culture flasks; and (iv) culturing the dissociated cells under adherent static conditions for at least one week. In some embodiments, the dissociated cells are cultured for 1-3 weeks.

In embodiments, the cells produced in any step may be cryopreserved. In embodiments, the cells are cryopreserved in a cryosolution. For example, the cryosolution may include CRYOSTEM™. In other embodiments, CRYOSTEM™ may be used as the cryosolution for freezing cell aggregates, and in other embodiments may be used for freezing the hESC sub-colonies.

In embodiments, the cells may be cryopreserved between days 14 to 120 of the differentiation process. In embodiments, the step of cryopreserving the cells from can be at or about the completion of the first time period.

In some embodiments of the methods of the disclosure, the methods comprise cryopreserving the population of retinal cells. In some embodiments, the cryopreservation comprises suspending the population of cells in a cryopreservation medium to form a cell suspension and storing the cell suspension at less than or equal to −80° C., or less than or equal to −140° C. In some embodiments, the cryopreservation comprises a cryopreservation medium suitable for administration to the eye of a subject. In some embodiments, the cryopreservation medium comprises a cryoprotective agent selected from the group consisting of glycerol, sucrose, dextran, and dimethyl sulfoxide (DMSO). In some embodiments, the cryopreservation medium comprises about 0.1% to about 40% of a cryoprotective agent. In some embodiments, the pharmaceutical composition comprises about 1% to about 10% of the cryoprotective agent.

In some embodiments, the cryopreservation medium comprises CRYOSTOR® CS2, CRYOSTOR® CS5, CRYOSTOR® CS10, or CRYOSTEM™. In some embodiments, the cryopreservation medium comprises 2% DMSO, 5% DMSO or 10% DMSO, or any range in between.

In some embodiments, the cryopreserved cells are thawed, and wherein the thawed cells are cultured. For example, the thawed cells are cultured in accordance with step (iii) above, or in other embodiments, the cells from step (iii) are cryopreserved after the third time period.

In some embodiments provided herein are methods for preparing a photoreceptor neural cell (PNC) composition for administration to a subject directly after thawing, the method including: (a) suspending the cells (e.g., the retinal cells comprising PNCs) prepared according to the methods herein in a cryopreservation media to form a cell suspension, (b) storing the cell suspension at a cryopreservation temperature, and (c) thawing the cryopreserved suspension. In embodiments, the cryopreservation media comprises one or more of adenosine, dextran-40, lactobionic acid, HEPES (N-(2-Hydroxyethyl) piperazine-N′-(2-ethanesulfonic acid)), sodium hydroxide, L-glutathione, potassium chloride, potassium bicarbonate, potassium phosphate, dextrose, sucrose, mannitol, calcium chloride, magnesium chloride, potassium hydroxide, sodium hydroxide, dimethyl sulfoxide (DMSO), or water. In embodiments, the cryosolution comprises methylcellulose. In embodiments, the cryosolution comprises dimethylsulfoxide (DMSO). In embodiments, the cryosolution comprises about 0.1% to about 20% DMSO. In other examples, the cryosolution includes about 0.1 to 100% DMSO, or from about 1 to about 100%, or from about 10 to 100% or from about 20 to 100%, or from about 50 to 100%, or from about 0.5 to 1%, or from about 0.1 to 1%, or from about 0.1 to about 10%.

The disclosure provides pharmaceutical compositions produced by the methods of the disclosure.

In embodiments, provided herein are pharmaceutical compositions for administrating to a subject, where the composition includes the mature PNCs (or PNCs) prepared according to any one of methods described herein, and in embodiments the pharmaceutical composition further includes a cryopreservation media. For example, in embodiments, the cryopreservation media comprises one or more of adenosine, dextran-40, lactobionic acid, HEPES (N-(2-Hydroxyethyl) piperazine-N′-(2-ethanesulfonic acid)), sodium hydroxide, L-glutathione, potassium chloride, potassium bicarbonate, potassium phosphate, dextrose, sucrose, mannitol, calcium chloride, magnesium chloride, potassium hydroxide, sodium hydroxide, dimethyl sulfoxide (DMSO), and water.

In embodiments, the composition includes cells (e.g. retinal cells comprising PNCs) at a concentration of about 1×10⁵ cells per mL to about 100×10⁶ cells per mL.

In embodiments, the pharmaceutical composition is stored at a volume of about 100 μl, to about mL, or about 250 μL, or about 600 μL. In embodiments, the cryopreservation media in the pharmaceutical composition comprises a cryosolution. In embodiments, the cryosolution comprises DMSO. In embodiments the cryosolution includes CRYOSTOR® CS2, CRYOSTOR® CS5, CRYOSTOR® CS10, or CRYOSTEM™.

In some embodiments, the pharmaceutical composition including the mature PNCs (or PNCs) prepared according to any one of methods described herein, comprises cells that express one or more markers selected from Crx, Recoverin, Cone Arrestin (CAR), or combinations thereof. In other embodiments, the pharmaceutical composition includes about 1%-30% RPE cells.

In other embodiments, the composition includes seeding the PNC on a scaffold, optionally biodegradable, preparing implants from the PNC containing scaffold, and cryopreserving implants for treating eye related diseases.

In other embodiments, provided herein are methods for treating eye-related diseases, e.g., for treating vision loss in a subject, where the method includes administering to the subject a therapeutically effective amount of the composition including the mature PNCs (or PNCs) prepared according to any one of methods described herein. In embodiments, the administering includes administering the composition into or adjacent to the retina of the subject. In embodiments, the administering is by injection, implantation, or transplantation. The disclosure provides methods of treating a subject with a vision condition, comprising administering a therapeutically effective amount of the pharmaceutical composition of the disclosure to an eye of the subject.

The disclosure provides methods of treating a subject with a vision condition, comprising administering a therapeutically effective amount of a pharmaceutical composition comprising a population of retinal cells, wherein (a) between about 10% to 70% of the cells in the population express Crx; (b) between about 3% to 90% of the cells in the population express Recoverin; (c) between about 3% to 90% of the cells in the population express CAR; and (d) between 0 to about 0.1% of the cells in the population express TRA-1-60 and/or SSEA5; wherein the composition is administered eye of the subject.

In some embodiments of the methods of the disclosure, the composition is administered via injection to the retina. In some embodiments the injection comprises intravitreal, subretinal or suprachoroidal injection of a suspension comprising the population of cells. In some embodiments, the methods comprise implantation of a scaffold comprising the population of cells. In some embodiments, the scaffold is implanted into the subretinal space.

In some embodiments of the methods of the disclosure, the vision condition comprises a neurodegenerative disease of the retina or damage to the retina. In some embodiments, the neurodegenerative disease of the retina comprises Stargardt's disease, diabetic retinopathy, macular degeneration, retinitis pigmentosa, Leber congenital amaurosis, cone-rod dystrophy, choroideremia, or X-linked retinoschisis. In some embodiments, the macular degeneration comprises age related macular degeneration.

Other aspects of the invention are disclosed infra.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1 is a schematic showing a bioretina differentiation flow chart, indicating the following at each stage: the major cell population, the inducing factors, and exemplary identity markers.

FIGS. 2A-2D are images showing the design of a study designed induce differentiation retinal cells from undifferentiated pluripotent cells (Bioret-Pro-01 Study design, also referred to as PR-44), indicating seeding densities and rotation velocities, incubation durations and IGF-1 concentrations that were tested in the study. FIG. 2A is an image showing a study with an initial hESC (human embryonic stem cell) density of 250,000 cells/mL and a rotation velocity of 35, 50 and 70 revolutions per minute (RPM). FIG. 2B is an image showing a study with an initial hESC density of 150,000 cells/mL and a rotation velocity of 35, 50 and 70 RPM. FIG. 2C is an image showing a study with an initial hESC density of 250,000 cells/mL and a rotation velocity of 40, 60 and 80 RPM. FIG. 2D is an image showing an initial hESC cell density of 150,000 cells/mL and a rotation velocity of 40, 60 and 80 RPM.

FIGS. 3A-3D are phase contrast images showing the aggregated morphology at the different culture conditions derived from the studies of FIGS. 2A-2D. All images are shown at the same scale.

FIG. 4 are images showing the estimation of cell amounts at Week #7. Numbers below the matrix (bottom panel) indicate squares containing aggregates, letters (A-D) labeling the bottom images correspond to the conditions shown in panels A-D of FIGS. 2A-2D, respectively.

FIG. 5 are images showing the estimation of cell amounts at Week #16. Numbers below the matrix indicate squares containing aggregates.

FIG. 6 is a pair of graphs showing glucose (bottom) and lactate (top) levels throughout the differentiation process.

FIG. 7 is a series of representations from confocal imaging of the cells. Week 16 retinal cells grown in the PBS Wheel were harvested and seeded on PDL laminin coated cover glasses, and immunostained for CRX (red) and Recoverin (green) 14 days post seeding.

FIG. 8 is a series of representations from confocal imaging of the cells. Week 16 retinal cells grown in PBS Wheel were harvested and seeded on PDL laminin coated cover glasses, and immunostained for Rhodopsin (red) and Cone Arrestin (green) 14 days post seeding.

FIG. 9 is a series of representations from confocal imaging of the cells. Week 8 and week 16 retinal cells grown in PBS Wheel or static control were harvested and seeded on PDL laminin coated cover glasses, and immunostained for Rhodopsin, HuCD, CRX, βTub3, CHX10, OTX2 (red) and Cone Arrestin, OTX2, Six6, Rhodopsin, and Recoverin are in the colors indicated in each image.

FIG. 10 is a table showing quantification of retinal marker expression throughout the differentiation process.

FIG. 11 is a table showing quantification of non-targeted retinal marker expression throughout the differentiation process.

FIG. 12 is a table showing quantification of marker expression of non-target cell populations throughout the differentiation process.

### DETAILED DESCRIPTION

Provided herein are, inter alia, are methods for differentiating human pluripotent stem cells to populations of retinal cells, such as photoreceptor neural cells (PNCs), in a dynamic culture, which may be adapted to a commercial scale process. Also provided herein are compositions comprising cells and cell populations obtained by the methods described herein. Also provided herein are methods for treating retinal diseases, including for example vision loss, using the cells and cell populations obtained by the methods described herein. Embodiments herein generally relate to methods, compositions of matter, and methods of treating eye diseases using neural retinal cells (NRCs) or photoreceptor neural cells (PNCs) obtained by the methods described herein.

The methods described herein provide certain advantages over other methods, including for example, 1) increased duration or timing (e.g., at least 4 months), where the undifferentiated pluripotent stem cells are seeded for differentiation as single cells and grown in suspension as cell aggregates for at least 14-18 weeks, 2) no isolation step is required, for example, where the targeted cells are typically isolated using known techniques, 3) the cells are grown in a dynamic culture as opposed a non-adherent or adherent static culture, and 4) the aggregate size of the aggregates of differentiating cells can be controlled by rotational velocity, which becomes important for commercial viability, as well as for homogeneity of the resulting cell population. Other advantages include increased efficiency (e.g., increased efficiency of the culturing process), increased quality of the cells, and increased percentages of viable cells.

As described herein, methods for generating retinal cells, for example photoreceptor neural cells, which may be capable of forming reconstructed retina with a high survivability and neural connectivity to the surrounding functional layers are provided. Moreover, the manufacturing process for the cells and compositions described herein may be compatible with large-scale production (e.g., in a closed system), with improvements to enable industrial manufacturing. The photoreceptor neural cells' clinically compatible characteristics were established using the protocol herein to generate positive identity assays for key markers of both rod and cone photoreceptor populations. In addition retinal cells populations produced using the methods described herein may comprise markers indicating the presence of additional retinal cell types, such as, but not limited retinal pigment epithelial (RPE) cells, glia, and precursors of photoreceptor neural cells.

Furthermore, the data herein indicated that a single-cell suspension of photoreceptor precursor cells has the potential to survive and mature post-transplantation (e.g., in a rodent model of retinal degeneration).

The methods described herein provide new opportunities for clinical (and commercial) production of photoreceptor cells in areas of large unmet needs, such as, but not limited to, retinitis pigmentosa, Stargardt's disease, and retinal detachments and tears.

After reading this description it will become apparent to one skilled in the art how to implement the present disclosure in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present disclosure as set forth herein.

Before the present technology is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description divided into various sections only for the reader's convenience and disclosure found in any section may be combined with that in another section. Titles or subtitles may be used in the specification for the convenience of a reader, which are not intended to influence the scope of the present disclosure.

### Definitions

As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or sub-value there between. Preferably, the term “about” when used with regard to an amount means that the amount may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a composition as described herein (including embodiments and examples).

As used herein, “treating” or “treatment” of a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean inhibiting the progression of the condition, disorder or disease, slowing the progression of the condition, disorder or disease temporarily, although in some instances, it involves halting the progression of the condition, disorder or disease permanently.

As used herein, the terms “treat” and “prevent” are not intended to be absolute terms. In various embodiments, treatment can refer to a 10%, 20%, 30%, 40%, 50%, 60%, 70%, 80%, 90%, or 100% reduction in the severity of an established disease, condition, or symptom of the disease or condition. In embodiments, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. In embodiments, references to decreasing, reducing, or inhibiting include a change of 10%, 20%, 30%, 40%, 50%, 60%, 70%, 80%, 90% or greater as compared to a control level and such terms can include but do not necessarily include complete elimination. In embodiments, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

An “effective amount” is an amount sufficient for a composition to accomplish a stated purpose relative to the absence of the composition (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug (e.g., the cells described herein) is an amount of the drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. For any composition described herein, the therapeutically effective or prophylactically effective amount can be initially determined from cell culture assays, and/or in vivo experiments in animal models. Target concentrations will be those concentrations of active composition(s) (e.g., cell concentration or number) that are capable of achieving the methods described herein, as measured using the methods described herein or known in the art.

An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any composition described herein, the therapeutically effective amount can be initially determined from cell culture assays. Target concentrations will be those concentrations of active composition(s) (e.g., cell concentration or number) that are capable of achieving the methods described herein, as measured using the methods described herein or known in the art.

As used herein, “implantation” or “transplantation” refers to the administration of a cell population into a target tissue using a suitable delivery technique, (e.g., using an injection device). Transplantation or implantation can refer to the administration of a cell population as a liquid, for example a liquid comprising the population of cells in suspension. Alternatively, implantation of transplantation can refer to the administration of a cell population to a target tissue when the cells are part of (contained within, or attached to) a device such as a scaffold.

“Patient” or “subject” refers to a living member of the animal kingdom suffering from or who may suffer from the indicated disorder. In embodiments, the subject is a member of a species comprising individuals who may naturally suffer from the disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human.

As used herein, a “patient in need thereof” or “subject in need thereof” refers to an animal or a human with a damage to or degeneration of the retina. Degeneration of the retina can be caused by genetic disorders (Stargardt's disease, Leber congenital amaurosis, retinitis pigmentosa, e.g.), or environmental factors (age, diet, smoking, trauma), as well as underlying pathologies such as diabetes (diabetic retinopathy).

A “pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compositions of the disclosure. One of skill in the art will recognize that other pharmaceutical excipients are useful in the present disclosure.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Biocompatible” refers to the ability of the material of device, such as a scaffold described herein, to elicit an appropriate host response in a specific situation, e.g. when implanted into the retina of a subject. For example, a scaffold that is biocompatible with the retina may show reduced or absent induction of the immune response upon implantation.

“Biodegradable” refers to a material, such as material of a scaffold described herein, to break down upon implanted into a subject. Biodegradable materials may break down over the course of hours, days, weeks, months or even years, depending upon the material.

As used herein, “dynamic culture” includes any method of culturing cells that is not static (e.g., in petri dishes). For example, and without limitation, dynamic culture includes culture on a rotator or shaker, wave bioreactor, stirred-tank bioreactor, rocker, air lift bioreactor, vertical wheel bioreactor, and fixed-bed bioreactor. Dynamic culture is conducted with intentional active motion to enhance mass transfer and mechanotransductive effects (e.g., using bioreactors) which often results in higher numbers of functional cells. For example, in dynamic differentiation processes, bioreactors directly apply mechanical forces to generate physiologic conditions and enhance differentiation towards a specific cell lineage. In dynamic culture, cells may also have a more homogenous environment, that diffusion alone cannot provide in static culture. For example, cells that are grown in the vessel periphery vs. vessel inner areas. Further, static culture may generate various biologically separate niches, as it sustains microenvironments with various cell densities, that are not sustainable in dynamic culture.

As used herein, by “pluripotent stem cells,” “pluripotent cells” or “stem cells” it is meant a cell that has the ability to differentiate into all types of cells in an organism. Pluripotent cells are capable of forming teratomas and of contributing to ectoderm, mesoderm, or endoderm tissues in a living organism. Examples of pluripotent stem cells are embryonic stem (ES) cells, embryonic germ stem (EG) cells, and induced pluripotent stem (iPS) cells, adult stem cells, mesenchymal stem cells and hematopoietic stem cells. Stem cells are also capable of remaining in an undifferentiated state (e.g., pluripotent or multipotent stem cells) for extended periods of time in culture until induced to differentiate into other cell types having a particular, specialized function (e.g., fully differentiated cells). Embryonic germ stem cells are cells that (a) can self-renew, (b) can differentiate to produce all types of cells in an organism, and (c) is derived from germ cells and germ cell progenitors, e.g. primordial germ cells, i.e. those that would become sperm and eggs. Embryonic germ cells (EG cells) are thought to have properties similar to embryonic stem cells as described above. Examples of methods of generating and characterizing EG cells may be found in, for example, U.S. Pat. No. 7,153,684; Matsui, Y., et al., (1992) Cell 70:841; Shamblott, M., et al. (2001) Proc. Natl. Acad. Sci. USA 98: 113; Shamblott, M., et al. (1998) Proc. Natl. Acad. Sci. USA, 95:13726; and Koshimizu, U., et al. (1996) Development, 122:1235, the disclosures of which are incorporated herein by reference.

NIC, also known as “niacinamide,” “nicotinamide,” or “NA,” is the amide derivative form of Vitamin B3 (niacin) which is thought to preserve and improve beta cell ftunction. NIC has the chemical formula C6H6N20. NIC is essential for growth and the conversion of foods to energy, and it has been used in arthritis treatment and diabetes treatment and prevention.

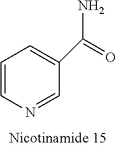

According to particular embodiments, the nicotinamide is a nicotinamide derivative or a nicotinamide mimic. The term “derivative of nicotinamide” as used herein denotes a compound which is a chemically modified derivative of the natural NA. In one embodiment, the chemical modification may be a substitution of the pyridine ring of the basic NA structure (via the carbon or nitrogen member of the ring), via the nitrogen or the oxygen atoms of the amide moiety.

When substituted, one or more hydrogen atoms may be replaced by a substituent and/or a substituent may be attached to a N atom to form a tetravalent positively charged nitrogen. Thus, the nicotinamide of the present invention includes a substituted or non-substituted nicotinamide. In another embodiment, the chemical modification may be a deletion or replacement of a single group, e.g., to form a thiobenzamide analog of NA, all of which being as appreciated by those versed in organic chemistry. The derivative in the context of the invention also includes the nucleoside derivative of NA (e.g., nicotinamide adenine). A variety of derivatives of NA are described, some also in connection with an inhibitory activity of the PDE4 enzyme (WO03/068233; WO02/060875; GB2327675A), or as VEGF-receptor tyrosine kinase inhibitors (WOO1/55114). For example, the process of preparing 4-aryl-nicotinamide derivatives (WO05/014549). Other exemplary nicotinamide derivatives are disclosed in WOO1/55114 and EP2128244.

Nicotinamide mimics include modified forms of nicotinamide, and chemical analogs of nicotinamide which recapitulate the effects of nicotinamide in the differentiation and maturation of RPE cells from pluripotent cells. Exemplary nicotinamide mimics include benzoic acid, 3-aminobenzoic acid, and 6-aminonicotinamide. Another class of compounds that may act as nicotinamide mimics are inhibitors of poly(ADP-ribose) polymerase (PARP). Exemplary PARP inhibitors include 3-aminobenzamide, Iniparib (BSI 201), Olaparib (AZD-2281), Rucaparib (AG014699, PF-01367338), Veliparib (ABT-888), CEP 9722, MK 4827, and BMN-673.

As used herein, “induced pluripotent stem cells” or “iPSCs” are cells that can be generated from somatic cells by genetic manipulation of somatic cells, e.g., by retroviral transduction of somatic cells such as fibroblasts, hepatocytes, gastric epithelial cells with transcription factors such as Oct-3/4, Sox2, c-Myc, and KLF4 (see Yamanaka S, Cell Stem Cell. 2007, 1 (1):39-49; Aoi T, et al., Generation of Pluripotent Stem Cells from Adult Mouse Liver and Stomach Cells. Science, 2008 Feb. 14; IH Park, Zhao R, West J A, et al. Reprogramming of human somatic cells to pluripotency with defined factors, Nature 2008; 451:141-146; K Takahashi, Tanabe K, Ohnuki M, et al. Induction of pluripotent stem cells from adult human fibroblasts by defined factors, Cell 2007; 131:861-872). Other embryonic-like stem cells can be generated by nuclear transfer to oocytes, fusion with embryonic stem cells or nuclear transfer into zygotes if the recipient cells are arrested in mitosis. In addition, iPSCs may be generated using non-integrating methods e.g., by using small molecules or RNA. Thus, iPSCs are cells that can (a) self-renew, (b) differentiate to produce all types of cells in an organism, and c) are derived from a somatic cell. iPS cells can have an ES cell-like morphology, growing as flat colonies with large nucleo-cytoplasmic ratios, defined borders and prominent nuclei. In addition, iPS cells express one or more key pluripotency markers known by one of ordinary skill in the art, including but not limited to Alkaline Phosphatase, SSEA3, SSEA4, SRY-box transcription factor 2 (Sox2), Oct-4, Nanog, TRA-1-60, TRA-1-81, teratocarcinoma-derived growth factor 1 (TDGF1), DNA methyltransferase 3 beta (Dnmt3b), forkhead box D3 (FoxD3), growth differentiation factor 3 (GDF3), cytochrome P450 family 26 subfamily A member 1 (Cyp26a1), telomerase reverse transcriptase (TERT), and ZFP42 zinc finger protein (zfp42). iPS cells may be generated by providing the cell with “reprogramming factors”, i.e. one or more, i.e. a cocktail, of biologically active factors that act on a cell to alter transcription, thereby reprogramming a cell to pluripotency. These reprogramming factors may be provided to the cells individually or as a single composition, that is, as a premixed composition, of reprogramming factors. The factors may be provided at the same molar ratio or at different molar ratios. The factors may be provided once or multiple times in the course of culturing the cells of the subject invention. Examples of methods of generating and characterizing iPS cells may be found in, for example, Application Nos. US20090047263, US20090068742, US20090191159, US20090227032, US20090246875, and US20090304646, the disclosures of which are incorporated herein by reference. Pluripotent stem cells express markers known to persons of skill in the art, including, but not limited to, TRA-1-60 antigen (TRA-1-60) and SSEA-5 glycan (SSEA5 or SSEA-5).

The term “embryonic stem cells” refers to embryonic cells that are capable of differentiating into cells of all three embryonic germ layers (i.e., endoderm, ectoderm and mesoderm), or remaining in an undifferentiated state. The term “embryonic stem cells” includes cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO 2006/040763) and embryonic germ (EG) cells which are obtained from the genital tissue of a fetus any time during gestation, preferably before 10 weeks of gestation. In embodiments, embryonic stem cells are obtained using well-known cell-culture methods. For example, human embryonic stem cells can be isolated from human blastocysts.

It is appreciated that commercially available stem cells can also be used in aspects and embodiments of the present disclosure. Human ES cells may be purchased from the NIH human embryonic stem cells registry, www.grants.nih.govstem_cells/or from other hESC registries. Suitable human ES cell lines will be apparent to the person of ordinary skill in the art, and are described in more detail below.

As used herein, “somatic cell” it is meant any cell in an organism that, in the absence of experimental manipulation, does not ordinarily give rise to all types of cells in an organism. In other words, somatic cells are cells that have differentiated sufficiently that they will not naturally generate cells of all three germ layers of the body, i.e. ectoderm, mesoderm and endoderm. For example, somatic cells include both neurons and neural progenitors, the latter of which may be able to self-renew and naturally give rise to all or some cell types of the central nervous system but cannot give rise to cells of the mesoderm or endoderm lineages.

As use herein, the term “retina” refers to the light sensitive layers of cells lining the back wall of the inside of the eye. Cells in the retina receive images through stimulation by light and transmit this information as electrical signals through the optic nerve to the brain. The retina and the optic nerve are considered outgrowths of the brain and are thus considered part of the central nervous system (CNS). The retina consists of retinal pigment epithelium (RPE) and the neural retina (NR), represented by interconnecting layers of specialized cells. From closest to farthest from the vitreous body, that is, from closest to the front exterior of the head towards the interior and back of the head, the retinal layers include the following (i) the inner limiting membrane comprised of Muller cell footplates, (ii) the nerve fiber layer containing axons of the ganglion cell nuclei, (iii) the ganglion cell layer, which contains nuclei of ganglion cells, the axons of which become the optic nerve fiber, (iv) the inner plexiform layer that contains the synapses between bipolar cell axons and the dendrites of the ganglion and amacrine cells, (v) the inner nuclear layer, which contains the nuclei and surrounding cell bodies (perikarva) of the bipolar cells, (vi) the outer plexiform layer, which contains projections of rods and cones ending in the rod spherule and cone pedicle, respectively, (vii) the outer nuclear layer, which contains cell bodies of rods and cones, (viii) the external limiting membrane, which separates the inner segment portions of the photoreceptors from their cell nucleus, (xi) the photoreceptor layer, and (x) the retinal pigment epithelium (RPE), which is a single layer of cuboidal cells. Some cells in the retina are photoreceptive, including rods, cones, and ganglion cells, which are directly sensitive to light. Rods primarily function in dim light to provide black-and-white vision. Cones primarily function in daytime vision and the perception of color. Photoreceptor is the photosensitive ganglion cell, is important for reflexive responses to bright daylight. The structure of the retinal, and retinal cell types, are described in US 20190085287, the contents of which are incorporated by reference herein in their entirety.

As used herein, “retinal cells” refers to all cell types found in the retina, in any combination. Retinal cells include, but are not limited to, neural retinal cells (NRCs), Muller glia cells, retinal pigmented epithelial (RPE) cells, or any combination thereof. The term “retinal cells” as used herein also encompasses “retinal progenitor cells,” or “RPCs.” The vertebrate retina develops from a domain in the anterior neural plate (the eye field). Bilateral optic vesicles form in this region, which develop into the optic cup through invagination. The retina is formed from the posterior wall of the optic cup. Retinal progenitor cells (RPCs) in the optic cup undergo active proliferation, producing the prospective RPE and NR layers of the retina. Retinal progenitor cells in the outer layer of the optic cup become RPE progenitors, while the inner layer become NR progenitors. RPCs are multipotent, and capable of producing the full range of retinal cell types when subject to appropriate conditions to induce differentiation.

As used herein “neural retinal cells,” neuronal retinal cells” and similar, abbreviated NRCs, refers to neuronal cells that arise from NR progenitors. There are six major types or neural retinal (NR) cells, all neurons, which include rod and cone photoreceptor cells, bipolar cells, horizontal cells, amacrine cells, and ganglion cells. Miller glial cells (macroglia), microglial cells (resident tissue-specific macrophages), astrocytes, and oligodendrocytes are non-neuronal cell that are integrated into the NR layer of the retina.

As used herein, “photoreceptor cells.” photoreceptor neural cells” or “PNC” and the like refers to a specialized type of neuroepithelial cell found in the retina that is capable of visual phototransduction. There are currently three known types of photoreceptor cells in mammalian eyes: rods, cones, and intrinsically photosensitive retinal ganglion cells. Rod and cone photoreceptor cells are the primary photoreceptor cells, while photosensitive retinal ganglion cells are thought to play a role in non-visual responses to light (e.g., circadian rhythms). Rod photoreceptor cells primarily mediate scotopic vision (i.e., under dim conditions) whereas cone photoreceptor cells primarily mediate to photopic vision (i.e., under bright conditions). Rod and cone photoreceptor cells have the same basic structure. Closest to the visual field is the outer segment, which contains a stack of membranous disks containing photopigments, followed by the inner segment, containing mitochondria, the nucleus, and finally the synaptic body.

“Photoreceptor cell precursors”, “precursors of photoreceptor neuronal cells,” and the refer to post-mitotic precursor cells which can differentiate into cone or rods photoreceptor cells, and that eventually express different photopigments. Photoreceptor cells, and photoreceptor cell precursors can express markers that include, but are not limited to cone-rod homeobox (Crx), Recoverin. and Cone Arrestin (CAR).

Bipolar cells are retinal interneurons, and provide one of the main pathways connecting the photoreceptor cells to the ganglion cells. Bipolar cells can be divided into rod and cone bipolar cells based on the type of photoreceptor cell with which the bipolar cell synapses.

Horizontal cells and amacrine cells are interneurons that are primarily responsible for lateral interactions within the retina. Amacrine cells receive inputs from bipolar cells, while horizontal cells receive inputs from photoreceptor cells. Amacrine cells operate in the inner plexiform layer, while horizontal cells operate in the outer plexiform layer. Both cell types are found in the inner nuclear layer of the retina.

Retinal ganglion cells propagate visual signals from the photoreceptor cells to the visual centers of the brain. Retinal ganglion cell axons project from the retina to the brain. There are a variety of subtypes of retinal ganglion cells, which can be classified by persons of skill in the art based on the size and morphology of the cell body and dendrites, as well as the extent of the dendritic field, for example the stratum of the inner plexiform layer to which the dendrites extend.

Glial cells, also called neuroglia, are non-neuronal cells that are located within the central and peripheral nervous system and provide physical and metabolic support to neurons. Muller glial cells are the major type of retinal glial cells. Without wishing to be bound by theory, it is thought that Muller glia are responsible for the maintain the structural and functional stability of retinal neural cells. This includes regulation of the extracellular environment, e.g. by neurotransmitter uptake, clearing of cellular debris, regulating ion levels, and storing glycogen, as well as providing electrical insulation and mechanical support for neural retinal cells.

As used herein, the “retinal pigment epithelium” “retinal pigment epithelial cells” or “RPEs” refers to a single layer of cuboidal cell, which is farthest layer of from the vitreous body of the eye, i.e. the RPE is between the NR layer and the choroid. RPE cells play an important role in maintaining visual function. RPE cells are phagocytic, and can engulf and eliminate photoreceptor outer segments. RPE cells help maintain the normal renewal of visual cells. RPE cells express a variety of markers during development, which can be divided into early, intermediate and late RPE markers. Early RPE markers include, but are not limited to melanocyte inducing transcription factor (MITF), tyrosinase related protein 1 (TYRP1), premelanosome protein (PMEL), and transmembrane protein with EGF like and two follistatin like domains (TMEFF2); intermediate RPE markers include, but are not limited to tyrosinase (TYR) and retinaldehyde binding protein (1RLBP1); and late RPE markers include, but are not limited to, retinoid isomerohydrolase RPE65 (RPE65), bestrophin 1 (BEST1), and retinal G protein coupled receptor (RGR).

As used herein, the “early eye field” refers to a domain in the anterior neural plate, established during gastrulation, that gives rise to the optic vesicles. The early eye field is a single, coherent group of cells whose identity can be determined through the expression of early eye field markers including, but not limited to, SIX homeobox 3 (Six3), SIX homeobox 6 (Six6), retinal homeobox gene 1 (Rx1), paired box 6 (Pax6), retinoid X receptor gamma (RXRγ), LIM homeobox 2 (LHX2) and retina and anterior neural fold homeobox (RAX). Early eye field cells differentiate into embryonic retinal cells, which in turn give rise retinal progenitor cells that differentiate into the various cell types found in the retina. Embryonic retinal cells express markers including, but not limited to, orthodenticle homeobox 2 (Otx2), Neuronal Differentiation 1 (NeuroD), B lymphocyte-induced maturation protein-1 (Blimpl), Transducin, Phosducin (PdC), RXRy and thyroid hormone receptor isoform (Tr-32), atonal bHLH transcription factor 7 (Atoh7) and insulin gene enhancer protein (ISI-1). Without wishing to be bound by theory, it is thought that retinal differentiation occurs as retinal progenitor cells undergo a series of competency changes in response to signals during development, so that RPCs at early developmental stages generate early retinal cell types (e.g., retinal ganglion cells), whereas late retinal progenitor cells generate the late cell types (e.g., Muller cells). However, as there is temporal overlap in the genesis of different retinal cell types, multiple retinal cell types can form concurrently, and retinal progenitor cells may be heterogenous.

Unless otherwise defined, all technical and scientific terms used herein have the same meaning as commonly understood by one of ordinary skill in the art to which this disclosure belongs. The terminology used in the description of the disclosure herein is for the purpose of describing particular embodiments only and is not intended to be limiting of the disclosure.

All publications, patent applications, patents and other references cited herein are incorporated by reference in their entireties.

Unless the context indicates otherwise, it is specifically intended that the various features of the disclosure described herein can be used in any combination. Moreover, the present disclosure also contemplates that in some embodiments of the disclosure, any feature or combination of features set forth herein can be excluded or omitted.

Methods disclosed herein can comprise one or more steps or actions for achieving the described method. The method steps and/or actions may be interchanged with one another without departing from the scope of the present invention. In other words, unless a specific order of steps or actions is required for proper operation of the aspect, the order and/or use of specific steps and/or actions may be modified without departing from the scope of the present invention.

### Methods of Producing Populations of Retinal Cells

The disclosure provides methods of producing populations of retinal cells from undifferentiated pluripotent stem cells. The methods comprise culturing populations of undifferentiated pluripotent stem cells in different combinations of growth factors and growth factor inhibitors, in a series of steps that induces differentiation of the undifferentiated pluripotent stem cells towards retinal cell fates. In an exemplary differentiate pathway, human embryonic stem cells (hESCs) are induced to differentiate into early eye field cells, which are induced to differentiate into embryonic retinal cells, which in turn are induced to differentiate into precursors and mature photoreceptor neuronal cells (PNCs). The resulting population of cells may contain a mixture of cell types. However, cells from the later stages of the pathway may predominate, and residual hESCs may be minimal or absent.

Without wishing to be bound by theory, it is thought that a composition comprising a mixed cell population may be a suitable therapeutic agent for vision disorders and diseases, particularly when delivered in combination with a scaffold, as the range of cell types increases the niches into which the cells can engraft when administered to a subject, and the increases of the number of fates that the cells can adopt upon administration.

In some aspects, provided herein are methods for obtaining a population of neural retinal cells, for example neural retinal cells, from undifferentiated pluripotent stem cells. In aspects, the method includes growing the undifferentiated pluripotent stem cells in a dynamic culture; seeding the undifferentiated pluripotent stem cells as single cells in a culture vessel; and culturing the undifferentiated pluripotent stem cells in the culture vessel under differentiation conditions to obtain the population of retinal cells, for example a population comprising NRCs. In embodiments of the invention, the culture vessel is a bioreactor, for example a PBS vertical wheel bioreactor.

### Methods of Inducing Retinal Cell Differentiation

In accordance with the present disclosure, human pluripotent stem cells (hPSCs) can be differentiated into different populations of cells through culture in a variety of different media comprising growth factors and growth factor inhibitors. In some embodiments, undifferentiated pluripotent stem cells are subjected to conditions sufficient for directed differentiation to produce a composition comprising a population of retinal cells, for example a population of cells comprising eye field cells, embryonic retinal cells, precursors of photoreceptor neuronal cells, mature photoreceptor neuronal cells, neuronal retina cells (NRCs) (e.g., retinal ganglion cells, horizontal neurons, amacrine neurons, rod bipolar cells, cone bipolar cells, rod photoreceptor cells, cone photoreceptor cells), Muller glia cells, retinal pigmented epithelial (RPE) cells, or any combination thereof. In some embodiments, the method comprises culturing a population of hPSCs in 3, 4, or 5 different culture media, each comprising a combination of growth factors and/or growth factor inhibitors, for 3, 4, or 5 time periods, under conditions sufficient to drive the population cells towards a target cell type, thereby producing a population of cells comprising the target cell type.

In some embodiments, the undifferentiated pluripotent stem cells are seeded in a culture vessel for differentiation as single cells and grown in suspension as cell aggregates for at least 14 weeks. In some embodiments, the undifferentiated pluripotent stem cells are seeded in a culture vessel for differentiation as single cells and grown in suspension as cell aggregates for 14-18 weeks. In examples, the cell concentration at seeding and vessel agitation velocity along the differentiation process may be adjusted to achieve optimal yield and quality. In further examples, the feeding frequency may be determined according to lactate and glucose levels along the differentiation process to achieve optimal yield and quality.

In some embodiments, the undifferentiated pluripotent stem cells are seeded at a density of 50,000 cell/mL to about 2,000,000 cells/mL, about 100,000 cells/mL to about 2,000,000 cells/mL, or about 200,000 cells/mL to about 500,000 cells/mL. In some embodiments, the cells are seeded at a density of about 200,000 cells/mL to about 300,000 cells/mL. In some embodiments, the cells are seeded at a density of about 150,000 cells/mL to about 250,000 cells/mL. In some embodiments the cells are seeded at a density of 250,000 cells/ml.

In some embodiments, the culture vessel is a bioreactor. In embodiments, the culture vessel is a vertical wheel bioreactor. In other embodiments, the bioreactor is a vertically-orientated bioreactor, e.g. VERTICAL-WHEEL® bioreactor (PBS Biotech, Camarillo, CA). In some embodiments, the bioreactor is a wave bioreactor. In some embodiments the bioreactor is a Gas Permeable Rapid Expansion bioreactor (e.g. G-REX®, Wilson Wolf Corp., Saint Paul, MN) bioreactor. The PBS wheel dynamic culture offers unique advantage to control the size of the aggregates while avoiding numerous steps of manual manipulation (such as seeding in microwells for aggregate formation, magnetic separation of undifferentiating cells, aggregate size filtration etc.) that otherwise prevent the translation of the technology to industrial grade. Although cell growth in bioreactors is done for many years with transformed mammalian cell lines, growing differentiated cells and moreover achieving controlled differentiation process within a bioreactor is not an obvious practice, as such differentiation needed a certain microenvironment that are generated within a niche in a 3D structure of a cell aggregate. A tight control is needed to keep the aggregates in the right size. Aggregates need to be small enough so that the differentiating factors will access the necessary cell layers but the velocity that control the aggregate size should maintain a minimal shear force to avoid cell damage. Moreover, the biological state of the undifferentiated cell origin may greatly affect the differentiation potential of the cells, and have impact on the final product yield, quality and cell viability.

In some aspects, culture conditions (e.g., optimal culture conditions) may be utilized in larger vessels for the differentiation process and scale up (e.g., to be commercially viable). In still other examples, the cell aggregates (e.g., organoids) described herein are grown in a dynamic suspension and directly differentiated into PNCs without the exclusion of pigmented cell aggregates (e.g., organoids) that contain retinal epithelial cells (RPEs). In examples, the cells after the differentiation process (e.g., mature PNCs or PNCs) contain about 1% to 30% RPEs. In examples, the cells after the differentiation process contain less than about 50% RPEs. In examples, the cells after the differentiation process contain less than about 40% RPEs. In examples, the cells after the differentiation process contain less than about 30% RPEs. In examples, the cells after the differentiation process contain between about 5% and about 30%, between about 10% and about 30%, or between about 20% and about 30% RPEs. In examples, the cells after the differentiation process contain between about 1% and about 25%, between about 1% and about 20%, between about 1% and about 15%, between about 1% and about 10%, or between about 1% and about 5% RPEs. Percentage of RPEs can be any value or subrange within the recited ranges, including endpoints.

In some embodiments, the aggregates have a size controlled by a rotation velocity of the culture vessel. In some embodiments, the size of the aggregates is from about 50 μm to about 900 μm in diameter. In some embodiments, the size of the aggregates is from about 100 m to about 800 μm in diameter. In some embodiments, the size of the aggregates is from about 50 μm to about 500 μm in diameter, from about 50 μm to about 300 μm in diameter, from about 100 μm to about 250 μm in diameter, from about 150 μm to about 350 μm in diameter, from about 300 μm to about 800 μm in diameter, from about 200 μm to about 600 μm in diameter, or from about 250 μm to about 450 μm in diameter. For example, as the aggregates grow in size, the velocity is increased to keep the aggregates in suspension and to generate controlled shear stress to control the size of the aggregates. For example, the velocity can start from about 30 rpms (revolutions per minute, also referred to as RPM), and can increase (e.g., ramp up) to about 50 rpms, about 60 rpms, about 70 rpms, about 80 rpms, about 90 rpms, or about 100 rpms. For example, the rotation velocity is increased during the differentiation process. The velocity may be increased by 1 rpm, 5 rpms, 10 rpms, 15 rpms, 20 rpms, or more at different stages throughout the differentiation process.

In embodiments, the aggregate size is between about 100 μm to about 800 μm in diameter, such as between 50 to 100 microns, between 50 to 150 microns, between 100 to 250 microns, or between 150 to 350 microns or between 250 to 450 microns. Size may be any value or subrange within the recited ranges, including endpoints.

In some embodiments, the culture conditions sufficient to produce aggregates of the desired size (e.g., 100 μm to 800 μm in diameter) comprise culturing the cells in a culture vessel. In some embodiments, the culture vessel is a is a vertical wheel bioreactor, a wave bioreactor, or a Gas Permeable Rapid Expansion bioreactor. In some aspects, the rotational velocity within the wheel bioreactor is increased over time thereby producing cellular aggregates that are 100 μm to 800 μm in diameter that are suspended in the bioreactor. In some aspects, the rotational velocity of the wheel bioreactor subjects the cellular aggregates to controlled shear stress to maintain optimal aggregate size. Without wishing to be bound by theory, it is thought that the culture conditions described herein allow for the uniform differentiation of all cells in the aggregate by allowing cells in the aggregate to have uniform exposure to differentiation factors in the cell culture medium.

Aggregates size can be controlled by the rotation velocity of the culture vessel (see Borys et al. Stem Cell Research & Therapy, 2021, 12:55, Overcoming bioprocess bottlenecks in the large-scale expansion of high-quality hiPSC aggregates in vertical-wheel stirred suspension bioreactors, doi.org/10.1186/s13287-020-02109-4). In embodiments, the size of the aggregates is from about 100 μm to about 800 μm. For example, the rotation velocity may be increased during the differentiation process. In exemplary embodiments, a 300-micron size aggregate of hESC derived embryoid bodies was found to be optimal for generation of CRX (Cone-Rod Homeobox) positive cells (see Yanai et al. 2013, Differentiation of Human Embryonic Stem Cells Using Size-Controlled Embryoid Bodies and Negative Cell Selection in the Production of Photoreceptor Precursor Cells, Tissue Engineering: Part C Volume 19, Number 10).

In some embodiments, the rotational velocity of the bioreactor is between 25 to 90 revolutions per minute (RPM, or rpms). In some embodiments, the rotational velocity of the bioreactor is between 35 to 80 RPM. In some embodiments, the initial rotational velocity, i.e. the rotational velocity at the start of the differentiation process, is about 30 to 50 RPM, and is increased to about 60 to 80 RPM by the end of the of the differentiation process. In some embodiments, the initial rotational velocity is about 30 to about 50 RPM, about 30 to about 45 RPM or about 35 to about 40 RPM. In some embodiments, the initial rotational velocity is about 30 RPM. In some embodiments, the initial rotational velocity is about 35 RPM. In some embodiments, the initial rotational velocity is about 40 RPM. In some embodiments, the initial rotational velocity is about 45 RPM. In some embodiments, the final rotational velocity, i.e. the rotational velocity at the end of the differentiation process, and before the cellular aggregates are optionally disaggregated and seeded in a static culture, is between about 60 to about 90 RPM. In some embodiments, the final rotational velocity is about 60 RPM. In some embodiments, the final rotational velocity is about 70 RPM. In some embodiments, the final rotational velocity is about 80 RPM. In some embodiments, the final rotational velocity is about 90 RPM.

In some embodiments, the methods comprise an initial rotational velocity of about 35 to 40 RPM during the first four weeks of the differentiation process, a second rotational velocity of about 50 to about 60 RPM during fifth to seventh weeks of the differentiation process, and third rotational velocity of about 70 to about 80 RPM during and after the eighth week of the differentiation process. The person of ordinary skill in the art will appreciate the rotational velocities provided herein are exemplary, e.g., suitable for use with the PBS wheel and similar wheel bioreactors to produce aggregates of between 100 and 800 microns in diameter. Alternative wheel bioreactors may different rotational velocities to achieve the desired aggregate size, which can be readily determined by persons of ordinary skill in the art.

In some embodiments, culturing the cells comprises the steps of i) culturing the undifferentiated pluripotent stem cells for a first time period under culture conditions sufficient to induce the stem cells to form cell aggregates (e.g., organoids), wherein the cell aggregates comprise cells expressing one or more early eye field markers selected from), retinal homeobox gene 1 (Rx1), paired box 6 (Pax6), retinoid X receptor gamma (RXRy), LIM homeobox 2 (LHX2) and retina and anterior neural fold homeobox (RAX).

In other embodiments, culturing the cells comprises culturing the cell aggregates from step i) above for a second time period under culture conditions sufficient to differentiate cell aggregates to cells expressing one or more Embryonic Retina markers selected from orthodenticle homeobox 2 (Otx2), neuronal differentiation 1 (NeuroD), B lymphocyte-induced maturation protein-1 (Blimp1), Transducin, Phosducin (PdC), RXRy and thyroid hormone receptor isoform (Tr-32), atonal bHLH transcription factor 7 (Atoh7) or insulin gene enhancer protein (Isl-1). In other embodiments, the differentiation process further includes culturing the cells expressing the one or more Embryonic Retina markers from step ii) above for a third time period under culture conditions sufficient to differentiate the cells to precursors of photoreceptor neural cells (PNCs) and mature P, wherein the PNCs express one or more proteins selected from Cone-rod homeobox (Crx), Recoverin, or Cone arrestin (CAR).

In other examples, at different stages of the differentiation process, different differentiating factors may be further introduced.

In some embodiments, the present disclosures relate to a method of producing a composition comprising a population of retinal cells. In some embodiments the method comprises (a) culturing a population of undifferentiated pluripotent stem cells in a first cell culture medium comprising Nicotinamide (NIC), a Wnt inhibitor such as rel-4-[(3aR,4S,7R,7aS)-1,3,3a,4,7,7a-Hexahydro-1,3-dioxo-4,7-methano-2H-isoindol-2-yl]-N-8-quinolinylbenzamide (IWRe), and Rock inhibitor (RI) for a first time period. In some embodiments, the RI is Y-27632 (trans-4-[(1R)-1-Aminoethyl]-N-4-pyridinylcyclohexanecarboxamide dihydrochloride). In some aspects, the undifferentiated pluripotent stem cells comprise human embryonic stem cells (hESCs) or human induced pluripotent stem cells (hiPSCs). In some aspects, the hESCs comprise HADC102 cells. In some aspects, the methods comprise culturing the population of cells produced in step (a) in a second cell culture medium comprising NIC and a Wnt inhibitor (e.g. IWRe) for a second time period. In some aspects, the methods comprise culturing the population of cells produced in step (b) in a third cell culture medium comprising NIC and Insulin-like growth factor 1 (IGF-1) for a third time period. In some aspects, the methods comprise (d) collecting the population of cells, thereby producing the composition comprising the population of retinal cells. In some aspects, the population of cells are cultured for at least 14 weeks in a culture vessel under conditions sufficient to produce cellular aggregates that are between about 100 μm to 800 μm in diameter.

In some embodiments, the method of producing a composition comprising a population of retinal cells comprises, before step (d), a step (i) comprising culturing the population of cells produced in step (c) in a fourth cell culture medium comprising IGF-1, NIC, and tert-Butyl (S)-{(2S)-2-[2-(3,5-difluorophenyl)acetamido]propanamido}phenylacetate (DAPT) for a fourth time period.

In some embodiments, the method of producing a composition comprising a population of retinal cells comprises, after step (i), a step (ii) comprising culturing the population of cells in step (i) in a fifth cell culture medium comprising IGF-1, Retinoic acid (RA), Taurine (TA), Brain-derived neurotropic factor (BDNF), and Neurorophin-4 (NT4) for a fifth time period.

In some embodiments, the first time period is 1-25 days, 1-15 days, 1-7 days, or 1-3 days. In some embodiments, the second time period is 1-30 days, 2-20 days, 11-20 days, or 3-14 days. In some embodiments, the third time period is 5 days to 14 weeks, 10 days to 3 weeks, 10 days to 8 weeks, or 2 weeks to 5 weeks. In some embodiments, the fourth time period is 2 weeks to 20 weeks, 2 weeks to 18 weeks, 2 weeks to 12 weeks, 4 weeks to 10 weeks, or 5 weeks to 6 weeks. In some embodiments, the fifth time period is 4 weeks to 18 weeks, 4 weeks to 12 weeks, 5 week to 9 weeks, 10 weeks to 25 weeks, 6 weeks to 24 weeks, 12 weeks to 18 weeks, 7 weeks to 16 weeks, or 7 weeks to 12 weeks. Time may be any value or subrange within the recited ranges, including endpoints.

In some examples, the first time period is between 1 to 7 days. In some examples, the second time period is between 2 to 20 days. In some embodiments, the third time period is between 10 days to 8 weeks. In some embodiments, the fourth time period is between 2 to 10 weeks. In some embodiments, the fifth time period is between 5 weeks to 18 weeks. In some embodiments, the fifth time period is between 9 weeks to 18 weeks.

In some embodiments, the first time period is 3-4 days, the second time period 10-11 days, the third time period is 20-21 days, the fourth time period is 7-8 days, and the fifth time period is 35-36 days. In other embodiments, the fifth time period is 63-64 days.

In some embodiments, the first time period is about 1-7 days, the second time period is about 2-20 days, the third time period is about 10 days to 8 weeks, the fourth time period is about 2 to 10 weeks, and the fifth time period is about 5 to 18 weeks.

In some embodiments, the methods comprise (i) collecting the cellular aggregates; (ii) dissociating the cellular aggregates to produce dissociated cells; and (iii) cryopreserving the dissociated cells using the methods described herein.

In alternative embodiments, the methods comprise, prior to step (d), (i) collecting the cellular aggregates; (ii) dissociating the cellular aggregates to produce dissociated cells; (iii) seeding the dissociated cells in tissue culture flasks; and (iv) culturing the dissociated cells under adherent static conditions for at least one week. In some embodiments, the dissociated cells are cultured for 1-5 weeks, 1-4 weeks, 1-3 weeks, or 1-2 weeks. In some embodiments, cells are collected from the tissue culture flasks and cryopreserved using the methods described herein.

The cellular aggregates can be collected by any methods known in the art including, but not limited to, centrifugation and filtration, or a combination thereof.

The cellular aggregates can be dissociated by any methods known in the art, including mechanical dissociation, chemical dissociation, and enzymatic digestion (e.g. with trypsin, such as TrpLE).

In some embodiments, for example where dissociated cells are cultured under adherent static conditions, the dissociated cells are seeded at a density of 50,000 cell/mL to about 2,000,000 cells/mL, about 100,000 cells/mL to about 2,000,000 cells/mL, or about 200,000 cells/mL to about 500,000 cells/mL. In some embodiments, the cells are seeded at a density of about 200,000 cells/mL to about 300,000 cells/mL. In some embodiments, the cells are reseeded at a density of about 150,000 cells/mL to about 250,000 cells/mL. In some embodiments the cells are seeded at a density of 250,000 cells/mL.

In some embodiments, the population of retinal cells retinal cells comprises early eye field cells, embryonic retinal cells, photoreceptor cells which may comprise precursors of photoreceptor neuronal cells (PNCs) or mature photoreceptor neuronal cells, or any combination thereof. In some embodiments, the population of retinal cells comprises neuronal retina cells (NRCs), Muller glia cells, retinal pigmented epithelial (RPE) cells, or any combination thereof. In some embodiments, the NRCs comprise PNCs, retinal ganglion cells, horizontal neurons, amacrine neurons, rod bipolar cells, cone bipolar cells, rod photoreceptor cells, cone photoreceptor cells or any combination thereof.

As described herein, in embodiments the pluripotent stem cells may be human embryonic stem cells (hESCs) or induced pluripotent stem cells (hiPSCs). In aspects, culturing the cells includes the steps of i) culturing the undifferentiated pluripotent stem cells, for a first time period under culture conditions sufficient to induce the stem cells to form cell aggregates (e.g., organoids), wherein the cell aggregates comprise cells expressing one or more Early Eye Field markers selected from Rx1, Pax6, RXRy, LHX2 and RAX. In other aspects, at different stages of the differentiation process, different differentiating factors may be further introduced.

In other aspects, culturing the cells further includes culturing the cell aggregates from step i) above for a second time period under culture conditions sufficient to differentiate cell aggregates to cells expressing one or more Embryonic Retina markers selected from Otx2, NeuroD, Blimp1, Transducin, Phosducin (PdC), RXRy and Tr-32, Atoh7 or Isl-1. In other embodiments, the differentiation process further includes culturing the cells expressing the one or more embryonic retina markers from step ii) above for a third time period under culture conditions sufficient to differentiate the cells to precursors of photoreceptor neural cells (PNCs) and mature photoreceptor neural cells, wherein the PNCs express one or more proteins selected from Crx, Recoverin, or Cone arrestin (CAR).

In an example differentiation protocol, the embryonic stem cells are differentiated using a first differentiating lineage and then further differentiated towards photoreceptor cells using IGF-1, retinoic acid (RA), BDNF, and NT4, to thereby produce the precursors and mature photoreceptor neuronal cells.

Additional contemplated differentiation agents include for example noggin, antagonists of FGF, (Dkk1 or IWR1e), nodal antagonists (Lefty-A), retinoic acid, taurine, GSK3b inhibitor (CHIR99021), notch inhibitor (DAPT), retinoic acid receptor (RAR) agonists or antagonists, agonists of FGF signaling pathway (aFGF, bFGF), agonists of the Hedgehog pathway (Shh), agonists of insulin growth factor pathway (IGF), agonists of the PI3-Kinase pathway, EGF pathway, BMP pathway, and Hippo pathway, and Rho-kinase (Rock) inhibitors.

Rho-kinase inhibitors (rho-associated protein kinase inhibitor or ROCK inhibitor) are a series of compounds that target rho kinase (ROCK) and inhibit the ROCK pathway. The ROCK inhibitor used in the methods described herein may be any ROCK inhibitor, including, but not limited to Y-27632 (trans-4-[(1R)-1-Aminoethyl]-N-4-pyridinylcyclohexanecarboxamide dihydrochloride), HA-1077 (Fasudil), HA-1100 (HydroxyFasudil), H-1152, 3-(4-Pyridyl)-1H-indole, N-(4-Pyridyl)-N′-(2,4,6-trichlorophenyl) urea, Aurothioglucose, LY294002 or a salt, base, ester or prodrug thereof.

Wnt inhibitors can be small molecules or proteins, including sFRP, Dkk, WIF, Wise/SOST, Cerberus, IGFBP, Shisa, Waif1, APCDD1, and Tiki1, that act to antagonize Wnt signaling, generally by preventing ligand-receptor interactions or Wnt receptor maturation. Exemplary Wnt inhibitors include rel-4-[(3aR,4S,7R,7aS)-1,3,3a,4,7,7a-Hexahydro-1,3-dioxo-4,7-methano-2H-isoindol-2-yl]-N-8-quinolinylbenzamide (IWRe) and IWP-4.

Such differentiation agents may be added at any stage of the differentiation procedure—e.g. prior to the first time period, during the first time period, during the second time period or following the second time period, during the third time period or following the third time period, during the fourth time period or following the fourth time period, during the fifth time period or following the fifth time period.

In some embodiments of the methods, the cells are cultured in the first time period with a Rock Inhibitor (RI, e.g., Y-27632), nicotinamide (NIC), and a Wnt inhibitor (e.g. IWRe1). In some embodiments, the Rock inhibitor can be at a concentration from about 0.01-100 μM, or from about 0.1 to 100 μM, or from about 1 to 100 μM, or from about 1 to 50 μM, or from about 1 to 40 μM, or from about 1 to 30 μM, or from about 1 to 20 μM, or from about 1 to 10 μM. In some embodiments, the Rock inhibitor can be at a concentration from about 1 to 10 μM. In some embodiments, the Rock inhibitor can be at a concentration of about 10 μM. The nicotinamide can be at a concentration from about 0.1 to 100 mM, or from about 1 to 100 mM, or from about 1 to 90 mM, or from about 1 to 80 mM, or from about 1 to 70 mM, or from about 1 to 60 mM, or from about 1 to 50 mM, or from about 1 to 40 mM, or from about 1 to 30 mM, or from about 1 to 20 mM. In some embodiments, the nicotinamide can be at a concentration from about 1 to 50 mM. In some embodiments, the Rock inhibitor can be at a concentration of about 10 mM. In other embodiments, the Wnt inhibitor can include any Wnt inhibitor, e.g., IWRe1. In some embodiments, the Wnt inhibitor can be at a concentration from about 0.01 to about 100 μM, or from about 0.5 to about 100 μM, or from about 0.5 to about 50 μM, or from about 0.5 to about 40 μM or from about 0.5 μM to about 30 μM, or from about 0.5 to about 20 μM. In some embodiments, the Wnt inhibitor can be at a concentration from about 0.5 to about 20 μM. In some embodiments the Wnt inhibitor can be at a concentration of about 3 μM. Concentrations may be any value or subrange within the recited ranges, including endpoints.

In some embodiments of the methods described herein, the cells are cultured for the second time period with NIC and a Wnt inhibitor. In some embodiments, the NIC can be at a concentration from about 0.1 to 100 mM, or from about 0.1 to 50 mM, or from about 1 to 100 mM, or from about 1 to 50 mM. In some embodiments, the nicotinamide can be at a concentration from about 1 to 50 mM. In some embodiments, the NIC can be at a concentration of about 10 mM. The Wnt inhibitor can include IWRe1 or Dkk1, sFRP, Dkk, WIF, Wise/SOST, Cerberus, IGFBP, Shisa, Waif1, APCDD1, and Tiki1. In some embodiments, the Wnt inhibitor includes IWRe1. The Wnt inhibitor can be at a concentration from about 0.01 to about 100 μM, or from about 0.5 to about 100 μM, or from about 0.5 to about 50 μM, or from about 0.5 to about 40 μM or from about 0.5 μM to about 30 μM, or from about 0.5 to about 20 μM. In some embodiments, the Wnt inhibitor can be at a concentration from about 0.5 to about 20 μM. In some embodiments the Wnt inhibitor can be at a concentration of about 3 μM. Concentrations may be any value or subrange within the recited ranges, including endpoints.

In some embodiments of the methods described herein, the cells are cultured in the third time period with NIC and Insulin growth factor-1 (IGF-1). In some embodiments, the nicotinamide can be at a concentration from about 0.1 to 100 mM, or from about 1 to 100 mM, or from about 1 to 90 mM, or from about 1 to 80 mM, or from about 1 to 70 mM, or from about 1 to 60 mM, or from about 1 to 50 mM, or from about 1 to 40 mM, or from about 1 to 30 mM, or from about 1 to 20 mM. In some embodiments, the nicotinamide can be at a concentration from about 1 to 50 mM. In some embodiments, the nicotinamide can be at a concentration of 10 mM. In some embodiments, the IGF-1 can be at a concentration from about can be at a concentration from about 0.01 to about 100 ng/mL, or from about 0.5 to about 100 ng/mL, or from about 0.5 to about 50 ng/mL, or from about 0.5 to about 40 ng/mL or from about 0.5 ng/mL to about 30 ng/mL, or from about 0.5 to about 20 ng/mL. In embodiments, the IGF-1 can be at a concentration from about can be at a concentration from about 0.5 to about 20 ng/mL In some embodiments the IGF-1 can be at a concentration of about 5 ng/mL. Concentrations may be any value or subrange within the recited ranges, including endpoints.

In further embodiments of the methods described herein, culturing the cells further includes differentiating for a fourth time period with NIC, IGF-1 and a Notch inhibitor. The nicotinamide can be at a concentration from about 0.1 to 100 mM, or from about 1 to 100 mM, or from about 1 to 90 mM, or from about 1 to 80 mM, or from about 1 to 70 mM, or from about 1 to 60 mM, or from about 1 to 50 mM, or from about 1 to 40 mM, or from about 1 to 30 mM, or from about 1 to 20 mM. In some embodiments, the nicotinamide can be at a concentration from about 1 to 50 mM, In some embodiments, the NIC can be at a concentration of about 10 mM. In some embodiments, the IGF-1 can be at a concentration from about 0.01 to about 100 ng/mL, or from about 0.5 to about 100 ng/mL, or from about 0.5 to about 50 ng/mL, or from about 0.5 to about 40 ng/mL or from about 0.5 ng/mL to about 30 ng/mL, or from about 0.5 to about 20 ng/mL. In some embodiments, the IGF-1 can be at a concentration from about 0.5 to about 20 ng/mL. In some embodiments, the IGF-1 can be at a concentration of about 5 ng/mL. In embodiments, the Notch inhibitor is DAPT, valproic acid, RO4929097, YO-1027, BT-GSI, CB-10, IMR-1, Psoralidin, avagacestat, J1051, and the like. In embodiments, the Notch inhibitor may be at a concentration from about 0.1 to 100 μM, or from about 1 to 100 μM, or from about 1 to 90 μM, or from about 1 to 80 μM, or from about 1 to 70 μM, or from about 1 to 60 μM, or from about 1 to 50 μM, or from about 1 to 40 μM, or from about 1 to 30 μM, or from about 1 to 20 μM. In some embodiments, the Notch inhibitor may be at a concentration from about 1 to 50 μM. In some embodiments, the Notch inhibitor may be at a concentration of about 10 μM. Concentrations may be any value or subrange within the recited ranges, including endpoints.

In further embodiments of the methods described herein, culturing the cells further includes differentiating for a fifth time period with IGF-1, retinoic acid (RA), taurine, BDNF and NT4. For example, the IGF can be at a concentration from about 1 to 100 ng/mL, or from about 1 to 50 ng/mL or from about 1 to 20 ng/mL, or from about 5 to 100 ng/mL, or from about 5 to 50 ng/mL, or from about 10 to 50 ng/mL, or from about 10.5 to 20 ng/mL. In some embodiments, the IGF can be at a concentration from about 0.5 to 20 ng/mL. In some embodiments, the IGF can be at a concentration of about 5 ng/mL. In other embodiments, the retinoic acid can be at a concentration from about 0.01 to 10 μM, or from about 0.01 to 5 μM, or from about 0.01 to 1 μM, or from about 0.1 to 10 μM, or from about 0.1 to 5 μM, or from about 0.1 to 20 μM. In other embodiments, the retinoic acid can be at a concentration from about 0.1 to 2 μM. In some embodiments, the RA can be at a concentration of about 0.5 μM. In other embodiments, the taurine (TA) may be at a concentration from about 0.1 to 1000 μM, or from about 1 μM to 1000 μM, or from about 10 to about 1000 μM, or from about 10 to 900 μM, or from about 10 to 800 μM, or from about 10 to 700 μM, or from about 10 to 600 μM, or from about 10 to 500 μM, or from about 10 to 400 μM, or from about 10 to 300 μM, or from about 10 to 200 μM, or from about 10 to 100 μM, or from about 10 to 400 μM. In other examples, the taurine (TA) may be at a concentration from about 10 to 400 μM. In some embodiments, the concentration of TA may be at a concentration of about 100 μM. In other embodiments, the BDNF can be at a concentration from about 1 to 500 ng/mL, or from about 1 to 400 ng/mL, or from about 1 to 300 ng/mL, or from about 1 to 200 ng/mL, or from about 1 to 100 ng/mL, or from about 2 to 500 mg/mL, or from about 2 to 400 ng/mL, or from 2 to 300 ng/mL, or from about 2 to 200 ng/mL. In other embodiments, the BDNF can be at a concentration from about 2 to 200 ng/mL. In some embodiments, the concentration of BDNF may be at a concentration of 20 ng/mL. In some embodiments, the NT4 can be at a concentration from about 1 to 500 ng/mL, or from about 1 to 400 ng/mL, or from about 1 to 300 ng/mL, or from about 1 to 200 ng/mL, or from about 1 to 100 ng/mL, or from about 2 to 500 mg/mL, or from about 2 to 400 ng/mL, or from 2 to 300 ng/mL, or from about 2 to 200 ng/mL. In some embodiments, the NT4 can be at a concentration from about 2 to 200 ng/mL. In some embodiments, the concentration of NT4 may be at a concentration of 20 ng/mL. Concentrations may be any value or subrange within the recited ranges, including endpoints.

In some embodiments, the present disclosure relates to a method of producing a composition of retinal cells comprising (a) culturing a population of undifferentiated pluripotent stem cells in a first cell culture medium comprising Nicotinamide (NIC) at a concentration of 10 mM, rel-4-[(3aR,4S,7R,7aS)-1,3,3a,4,7,7a-Hexahydro-1,3-dioxo-4,7-methano-2H-isoindol-2-yl]-N-8-quinolinylbenzamide (IWRe) at a concentration of 3 μm, and Rock inhibitor (RI) at a concentration of 10 μm for at least one day. In some embodiments, the method comprises (b) culturing the population of cells produced in step (a) in a second cell culture medium comprising NIC at a concentration of 10 mM, and IWRe at a concentration of 3 μm for at least 2 days. In some embodiments, the method comprises (c) culturing the population of cells produced in step (b) in a third cell culture medium comprising NIC at a concentration of 10 mM and Insulin-like growth factor 1 (IGF-1, or IGF1) at a concentration of 5 ng/mL for at least 10 days. In some embodiments, the method comprises (d) collecting the population of cells, thereby producing the composition comprising the population of retinal cells. In some embodiments, the populations of cells in are cultured for at least 14 weeks in a culture vessel under conditions sufficient to produce aggregates that are 100 μm to 800 μm in diameter.

In other embodiments, the present disclosure relates to a method of producing a composition of retinal cells comprising (a) culturing a population of undifferentiated pluripotent stem cells in a first cell culture medium comprising Nicotinamide (NIC) at a concentration of 10 mM, IWRe at a concentration of 3 μm, and Rock inhibitor (RI) at a concentration of 10 μm for at least one day. In some embodiments, the method comprises (b) culturing the population of cells produced in step (a) in a second cell culture medium comprising NIC at a concentration of 10 mM, and IWRe at a concentration of 3 m for at least 2 days. In some embodiments, the method comprises (c) culturing the population of cells produced in step (b) in a third cell culture medium comprising NIC at a concentration of 10 mM and Insulin-like growth factor 1 (IGF-1) at a concentration of 5 ng/mL for at least 10 days. In some embodiments, the method comprises (d) culturing the population of cells produced in step (c) in a fourth cell culture medium comprising IGF-t at a concentration of 5 ng/mL, NIC at a concentration of 10 mM, and DAPT at a concentration of 10 μM for at least 2 weeks. In some embodiments, the method comprises (e) collecting the population of cells, thereby producing the composition comprising the population of retinal cells. In some embodiments, the populations of cells in are cultured for at least 14 weeks in a culture vessel under conditions sufficient to produce aggregates that are 100 μm to 800 μm in diameter.

In other embodiments, the present disclosure relates to a method of producing a composition of retinal cells comprising (a) culturing a population of undifferentiated pluripotent stem cells in a first cell culture medium comprising Nicotinamide (NIC) at a concentration of 10 mM, IWRe at a concentration of 3 μm, and Rock inhibitor (RI) at a concentration of 10 μm for at least one day. In some embodiments, the method comprises (b) culturing the population of cells produced in step (a) in a second cell culture medium comprising NIC at a concentration of 10 mM, and IWRe at a concentration of 3 μm for at least 2 days. In some embodiments, the method comprises (c) culturing the population of cells produced in step (b) in a third cell culture medium comprising NIC at a concentration of 10 mM and Insulin-like growth factor 1 (IGF-1) at a concentration of 5 ng/mL for at least 10 days. In some embodiments, the method comprises (d) culturing the population of cells produced in step (c) in a fourth cell culture medium comprising IGF-t at a concentration of 5 ng/mL, NIC at a concentration of 10 mM, and DAPT at a concentration of 10 μM for at least 2 weeks. In some embodiments, the method comprises (e) culturing the population of cells produced in step (d) in a fifth cell culture medium comprising IGF-1 at a concentration of 5 ng/mL, Retinoic acid (RA) at a concentration of 0.5 μM, Taurine (TA) at a concentration of 100 μM, Brain-derived neurotropic factor (BDNF) at a concentration of 20 ng/mL, and Neurorophin-4 (NT4) at a concentration of 20 ng/mL for at least 5 weeks. In some embodiments, the method comprises (f) collecting the population of cells, thereby producing the composition comprising the population of retinal cells. In some embodiments, the populations of cells in are cultured for at least 14 weeks in a culture vessel under conditions sufficient to produce aggregates that are 300 μm to 800 μm in diameter.

In some embodiments, the methods described herein result in a population of cells wherein at least 50% of cells are viable, at least 60% of cells are viable, at least 70% of cells are viable, at least 80% of cells in the population are viable, at least 90% of cells in the population are viable, or at least 99% of cells in the population are viable. In some embodiments, at least 60% of cells in the population are viable. In some embodiments, at least 70% of cells in the population are viable. In some embodiments, at least 80% of cells in the population are viable. In some embodiments, at least 90% of cells in the population are viable. In some embodiments, at least 95% of cells in the population are viable. In some embodiments between about 50% to about 99% of cells in the population are viable, between about 60% to about 90% of cells are viable, between about 70% to about 90% of cells in the population are viable, between about 70% to about 80% of cells in the population of cells are viable, or between about 80% of cells to about 95% of cells in the population are viable. In some embodiments, between about 85% to about 95% of cells in the population are viable. In some embodiment, between about 50% to about 70% of cells in the population are viable. In some embodiments, between about 60% to about 80% of cells in the population are viable. Cell viability can be determined by any suitable assay known in the art, including vital dye based assays (e.g., MitoTracker Red, TMRE, Calcein and esterase based assays and the like).

Suitable basal cell culture media for culturing cell populations will be known to persons of ordinary skill in the art, and include commercially available basic media (i.e. a chemically defined medium or CDM), for example NUTRISTEM® (without TGFbeta and FGF2 for ESC differentiation, with TGFbeta and FGF2 for ESC expansion), NEUROBASAL™, KO-DMEM, DMEM, DMEM/F12, CELLGRO™ Stem Cell Growth Medium, or X-VIVO™. The basic medium may be supplemented with a variety of agents as known in the art dealing with cell cultures. The following is a non-limiting reference to various supplements that may be included in the culture to be used in accordance with the present disclosure: serum or with a serum replacement containing medium, such as, without being limited thereto, knock out serum replacement (KOSR), NUTRIDOMA-CS, TCH™, N2, N2 derivative, or B27 or a combination; an extracellular matrix (ECM) component, such as, without being limited thereto, fibronectin, laminin, collagen and gelatin. In some embodiments, the cell culture medium comprises a chemically defined medium (CDM) supplemented with N2, B27, or a combination thereof, optionally supplemented (with BrainPhys™, The ECM may then be used to carry the one or more members of the TGFI3 superfamily of growth factors; an antibacterial agent, such as, without being limited thereto, L-glutamine, beta mercaptoethanol, penicillin and streptomycin; and non-essential amino acids (NEAA), neurotrophins which are known to play a role in promoting the survival of SCs in culture, such as, without being limited thereto, BDNF, NT3, NT4.

In some embodiments, the present disclosure relates to a pharmaceutical composition produced by any of the above methods.

In some embodiments, the present disclosure relates to a method of treating a subject with a vision condition comprising a therapeutically effect amount of the pharmaceutical composition produced by any of the above methods.

### Cryopreservation of Retinal Cells

In some embodiments of the methods described herein, the population of retinal cells are cryopreserved and ready for administration to a subject upon thawing. In embodiments, the population of retinal cells are cryopreserved in a cryopreservation medium. In embodiments, the cryopreservation medium includes a cryoprotective agent, for example glycerol, sucrose, dimethyl sulfoxide (DMSO), or other suitable cryoprotective agent. In embodiments, the cryoprotective agent includes glycerol. In embodiments, the cryoprotective agent includes sucrose. In embodiments, the cryoprotective agent includes DMSO. In embodiments, the cryoprotective agent includes dextran.

In some embodiments, the cells are in a suspension when cryopreserved. For example, the cellular aggregates are dissociated, optionally the dissociated cells are cultured in static culture conditions, followed by filtration and/or centrifugation and resuspension in a cryopreservation medium.

In alternative embodiments, the cells are disposed upon or contained within a scaffold, and both the cells and the scaffold are cryopreserved.

In some embodiments, the cryopreservation medium includes about 0.1% to about 40% of the cryoprotective agent. In embodiments, the cryopreservation medium includes about 0.1% to about 30% of the cryoprotective agent. In embodiments, the cryopreservation medium includes about 0.10% to about 20% of the cryoprotective agent. In embodiments, the cryopreservation medium includes about 0.10% to about 10% of the cryoprotective agent. In embodiments, the cryopreservation medium includes about 0.1% to about 5% of the cryoprotective agent. In embodiments, the cryopreservation medium includes about 1% to about 40% of the cryoprotective agent. In embodiments, the cryopreservation medium includes about 1% to about 30% of the cryoprotective agent. In embodiments, the cryopreservation medium includes about 1% to about 20% of the cryoprotective agent. In embodiments, the cryopreservation medium includes about 1% to about 10% of the cryoprotective agent. In embodiments, the cryopreservation medium includes about 1% to about 5% of the cryoprotective agent. The percentage may be measured as weight of cryoprotective agent per volume of medium. The percentage may be measured as volume of cryoprotective agent per volume of medium. The percentage may be any value or subrange within the recited ranges, including endpoints.

In some embodiments, the cryopreservation comprises CRYOSTEM™, CRYOSTOR® CS2, CRYOSTOR® CS5, CRYOSTOR® CS10.

In other embodiments, the cells can be cryopreserved during the differentiation process. For example, the cells from step ii), e.g., culturing for a second time period to differentiate to cells expressing embryonic retina markers, can be cryopreserved, e.g., between days 14 to 120. The cells can be cryopreserved in a cryosolution including, for example, CRYOSTEM™, CRYOSTOR® CS2, CRYOSTOR® CS5, CRYOSTOR® CS10. In embodiments, the cryosolution may be specialized in freezing cell aggregates and the cryosolution comprises CRYOSTEMm.

In some embodiments, the cryopreservation comprises a cryopreservation medium suitable for administration to the eye of a subject.

In some embodiments, the cells can be cryopreserved after the first, second, third, fourth, or fifth time period. In some embodiments, a therapeutically effective amount of the cryopreserved cells can be thawed, and the thawed cells are immediately administered to an eye of a subject with a vision condition. In some embodiments, a therapeutically effective amount of the cryopreserved cells can be thawed, and the thawed cells are cultured prior to being administered to an eye of a subject with a vision condition. In further embodiments, the cryopreserved cells are thawed, and the thawed cells are then further cultured in accordance with any of the methods described herein.

In other embodiments, the cells can be cryopreserved after the second time period to differentiate to cells expressing embryonic retina markers at or about the completion of the first time period. In other embodiments, the cells can be cryopreserved after the third time period to differentiate to cells expressing precursors of photoreceptor neural cells (PNCs) and mature photoreceptor neural cells. For example, the cryopreserved cells are thawed and cultured in any remaining steps of the method.

In other embodiments described herein are methods for preparing a retinal cell composition (e.g., a composition comprising PNCs) for administration to a subject directly after thawing. For example, the method includes (a) suspending the cells prepared according to the methods herein in a cryopreservation media to form a cell suspension, (b) storing the cell suspension at a cryopreservation temperature, and (c) thawing the cryopreserved suspension. For example, the cryopreservation media can include one or more of adenosine, dextran-40, lactobionic acid, HEPES (N-(2-Hy droxyethyl) piperazine-N′-(2-ethanesulfonic acid)), sodium hydroxide, L-glutathione, potassium chloride, potassium bicarbonate, potassium phosphate, dextrose, sucrose, mannitol, calcium chloride, magnesium chloride, potassium hydroxide, sodium hydroxide, dimethyl sulfoxide (DMSO), or water.

In some embodiments the cryopreservation temperature is less than or equal to −80° C., or less than or equal to −140° C. In some embodiments of the methods described herein, the population of cryopreserved retinal cells is at a concentration of 1×10⁵ cells per mL to about 100×10⁶ cells per mL.

In some embodiments of the methods described herein, the population of cryopreserved retinal cells is at a concentration of 2×10⁶ cells per mL to about 5×10⁶ cells per mL. In some embodiments of the methods described herein, the population of cryopreserved retinal cells are stored at a volume of about 100 μL to about 1 mL. In some embodiments, the cryopreserved retinal cells are stored at a volume of about 250 μL. In some embodiments, the cryopreserved retinal cells are stored at a volume of about 600 μL.

### Methods for Expanding and Maintaining Pluripotent Cells

Provided herein are methods for expanding and maintaining human embryonic stem cells (hESCs), for example HADC102 cells, in an undifferentiated, pluripotent state. In some embodiments, the methods comprise the steps of (a) coating tissue culture flasks with LN521 for static expansion human embryonic stem cells in NutriStem® growth media supplemented with TGFbeta and FGF2, and (b) culturing the adherent hESCs for a period of time. In some embodiments, hESCs are expanded further by repeating steps (a) and (b). In some embodiments, thee methods comprise step (c) harvesting the cultured human embryonic stem cells produced by static expansion using TrypLE Select. The harvested hESCs are then differentiated using the methods described herein.

Provided herein are methods for expanding and maintaining human embryonic stem cells (hESCs), for example HADC102 cells, in an undifferentiated, pluripotent state, the method comprising the steps of (a) simultaneously combining human embryonic stem cells and an extracellular matrix component (ECM) in growth media in tissue culture flasks for static expansion, and (b) culturing the adherent hESCs for a period of time.

In an aspect, provided herein are methods for expanding and maintaining human embryonic stem cells (hESCs) in an undifferentiated, pluripotent state, the method comprising the steps of (a) simultaneously combining human embryonic stem cells and an extracellular matrix component (ECM) in growth media in tissue culture flasks for static expansion, and (b) culturing the adherent hESCs for a period of time. In some embodiments, the growth medium comprises NutriStem® supplemented with TGFbeta and FGF2.

In some embodiments, the cultured human embryonic stem cells of the static expansion are harvested non-enzymatically using ReLeSR™ and cultured in mTeSR™ plus media on iMatrix-511 coated vessels. In some embodiments, hESCs are expanded further by repeating steps (a) and (b).

In some embodiments, the cultured human embryonic stem cells of the static expansion are harvested and further differentiated.

In an aspect, provided herein are methods for expanding and maintaining human embryonic stem cells (hESCs) in an undifferentiated, pluripotent state, the method comprising the steps of (a) simultaneously combining human embryonic stem cells, an extracellular matrix component (ECM), and a microcarrier in growth media to form a suspendable expansion complex, and (b) culturing the suspendable expansion complex for a period of time.

In some embodiments, the cultured human embryonic stem cells of the suspendable expansion complex are harvested and expanded further by repeating steps (a) and (b).

In some embodiments, the cultured human embryonic stem cells of the suspendable expansion complex are harvested and further differentiated.

Human embryonic stem cells can be isolated from human blastocysts. Human blastocysts are typically obtained from human in vivo preimplantation embryos or from in vitro fertilized (IVF) embryos. Alternatively, a single cell human embryo can be expanded to the blastocyst stage. For the isolation of human ES cells the zona pellucida is removed from the blastocyst and the inner cell mass (ICM) is isolated by a procedure in which the trophectoderm cells are lysed and removed from the intact ICM by gentle pipetting. The ICM is then plated in a tissue culture flask containing the appropriate medium which enables its outgrowth. Following 9 to 15 days, the ICM derived outgrowth is dissociated into clumps either by a mechanical dissociation or by an enzymatic degradation and the cells are then re-plated on a fresh tissue culture medium. Colonies demonstrating undifferentiated morphology are individually selected by micropipette, mechanically dissociated into clumps, and re-plated. Resulting ES cells are then routinely split every 4-7 days. For further details on methods of preparation human ES cells, see Reubinoff et al. Nat Biotechnol 2000, May: 18(5): 559; Thomson et al., [U.S. Pat. No. 5,843,780; Science 282: 1145, 1998; Curr. Top. Dev. Biol. 38: 133, 1998; Proc. Natl. Acad. Sci. USA 92: 7844, 1995]; Bongso et al., [Hum Reprod 4: 706, 1989]; and Gardner et al., [Fertil. Steril. 69: 84, 1998].

In addition, ES cells can be obtained from other species, including mouse (Mills and Bradley, 2001), golden hamster [Doetschman et al., 1988, Dev Biol. 127: 224-7], rat [Iannaccone et al., 1994, Dev Biol. 163: 288-92], rabbit [Giles et al. 1993, Mol Reprod Dev. 36: 130-8; Graves & Moreadith, 1993, Mol Reprod Dev. 1993, 30 36: 424-33], several domestic animal species [Notarianni et al., 1991, J Reprod Fertil Suppl. 43: 255-60; Wheeler 1994, Reprod Fertil Dev. 6: 563-8; Mitalipova et al., 2001, Cloning. 3: 59-67] and non-human primate species (Rhesus monkey and marmoset) [Thomson et al., 1995, Proc Natl Acad Sci USA. 92: 7844-8; Thomson et al., 1996, Biol Reprod. 55: 254-9].

Extended blastocyst cells (EBCs) can be obtained from a blastocyst of at least nine days post fertilization at a stage prior to gastrulation. Prior to culturing the blastocyst, the zona pellucida is digested [for example by Tyrode's acidic solution (Sigma Aldrich, St Louis, MO, USA)] so as to expose the inner cell mass. The blastocysts are then cultured as whole embryos for at least nine and no more than fourteen days post fertilization (i.e., prior to the gastrulation event) in vitro using standard embryonic stem cell culturing methods.

Another method for preparing ES cells is described in Chung et al., Cell Stem Cell, Volume 2, Issue 2, 113-117, 7 Feb. 2008. This method comprises removing a single cell from an embryo during an in vitro fertilization process. The embryo is not destroyed in this process.

EG (embryonic germ) cells are prepared from the primordial germ cells obtained from fetuses of about 8-11 weeks of gestation (in the case of a human fetus) using laboratory techniques known to anyone skilled in the arts. The genital ridges are dissociated and cut into small portions which are thereafter disaggregated into cells by mechanical dissociation. The EG cells are then grown in tissue culture flasks with the appropriate medium. The cells are cultured with daily replacement of medium until a cell morphology consistent with EG cells is observed, typically after 7-30 days or 1-4 passages. For additional details on methods of preparation human EG cells see Shamblott et al., [Proc. Natl. Acad. Sci. USA 95: 13726, 1998] and U.S. Pat. No. 6,090,622.

Yet another method for preparing ES cells is by parthenogenesis. The embryo is also not destroyed in the process.

The cells may be expanded in suspension, with or without a microcarrier, or in a monolayer. The expansion of the mixed population of cells in monolayer cultures or in suspension culture may be modified to large scale expansion in bioreactors or multi/hyper stacks by methods well known to those versed in the art.

According to some embodiments, the expansion phase is effected for at least one to 20 weeks, for example at least one week, at least 2 weeks, at least 3 weeks, at least 4 weeks, at least 5 weeks, at least 6 weeks, at least 7 weeks, at least 8 weeks, at least 9 weeks or even 10 weeks. In embodiments, the expansion phase is effected for 1 week to 10 weeks, such 2 weeks to 10 weeks, 3 weeks to 10 weeks, 4 weeks to 10 weeks, or 4 weeks to 8 weeks. The time period may be any value or subrange within the recited ranges, including endpoints.

According to still other embodiments, the expansion phase is effected until a suitable lactate concentration in the cell culture medium, and/or precent confluence is achieved. Percent confluence is the percentage of the culture vessel surface area that appears covered by a layer of cells when observed by microscopy. In some embodiments, the undifferentiated pluripotent stem cells are cultured until the lactate concentration in the cell culture medium is between about 1.0 to 13.0 mM, or between about 1.5-12.5 mM. In some embodiments, cells are cultured until the lactate concentration in the cell culture medium is between about 1.68-12.29 mM. In some embodiments, the percent confluence is between 5% and 85%.

According to still other embodiments, the mixed population of cells are passaged at least one time during the expansion phase, at least twice during the expansion phase, at least three times during the expansion phase, at least four times during the expansion phase, at least five times during the expansion phase, at least six times during the expansion phase, or at least seven times during the expansion phase.

When cells are collected enzymatically, it is possible to continue the expansion for more than 8 passages, more than 9 passages and even more than 10 passages (e.g. 11-15 passages). The number of total cell doublings can be increased to greater than 30, e.g. 31, 32, 33, 34 or more. (See international patent application publication number WO 2017/021973, incorporated herein by reference in its entirety).

An extracellular matrix (ECM) is a three-dimensional network consisting of extracellular macromolecules and minerals, such as collagen, enzymes, glycoproteins and hydroxyapatite that provide structural and biochemical support to surrounding cells. Because multicellularity evolved independently in different multicellular lineages, the composition of ECM varies between multicellular structures; however, cell adhesion, cell-to-cell communication and differentiation are common functions of the ECM.

The animal extracellular matrix includes the interstitial matrix and the basement membrane. Interstitial matrix is present between various animal cells (i.e., in the intercellular spaces). Gels of polysaccharides and fibrous proteins fill the interstitial space and act as a compression buffer against the stress placed on the ECM. Basement membranes are sheet-like depositions of ECM on which various epithelial cells rest. Each type of connective tissue in animals has a type of ECM: collagen fibers and bone mineral comprise the ECM of bone tissue; reticular fibers and ground substance comprise the ECM of loose connective tissue; and blood plasma is the ECM of blood.

Suitable extracellular matrix components for use within the scope of the present disclosure may include, but are not necessarily limited to, Matrigel®, vitronectin, gelatin, collagen I, collagen IV, laminin (e.g. laminin 521), fibronectin poly-D-lysine, their derivatives, or a combination thereof. In specific embodiments, the human laminin is human laminin 511 E8 fragment.

In some embodiments, the microcarriers may comprise one or more of polystyrene, cross-linked dextran, magnetic particles, microchips, cellulose, hydroxylated methacrylate, collagen, gelatin, polystyrene, plastic, glass, ceramic, or silicone. In some embodiments, the microcarriers are composed of polystyrene, surface-modified polystyrene, chemically modified polystyrene, cross-linked dextran, cellulose, acrylamide, collagen, alginate, gelatin, glass, DEAE-dextran, or a combination thereof. In some embodiments, the microcarrier is composed of polystyrene. In some embodiments, the microcarrier is composed of surface-modified polystyrene. In some embodiments, the microcarrier is composed of chemically modified polystyrene. In some embodiments, the microcarrier is composed of cross-linked dextran. In some embodiments, the microcarrier is composed of cellulose. In some embodiments, the microcarrier is composed of acrylamide. In some embodiments, the microcarrier is composed of collagen. In some embodiments, the microcarrier is composed of alginate. In some embodiments, the microcarrier is composed of gelatin. In some embodiments, the microcarrier is composed of glass. In some embodiments, the microcarrier is composed of DEAE-dextran. In some embodiments, the microcarriers are not coated.

In some embodiments, the microcarriers are coated. In embodiments, the microcarriers may be coated with Matrigel®, laminin, vitronectin, collagen, their derivatives, or a combination thereof. In embodiments, the microcarriers may be coated by poly-lysine, poly-L-lysine, poly-D-lysine, fibronectin, tenascin, dextran, a peptide, or a combination thereof. In some embodiments, the microcarrier is coated with laminin. In some embodiments, the microcarrier is coated with Matrigel®. In some embodiments, the microcarrier is coated with collagen. In some embodiments, the s microcarrier is coated with poly-lysine. In some embodiments, the microcarrier is coated with poly-L-lysine. In some embodiments, the microcarrier is coated with poly-D-lysine. In some embodiments, the microcarrier is coated with vitronectin. In some embodiments, the microcarrier is coated with fibronectin. In some embodiments, the microcarrier is coated with tenascin. In some embodiments, the microcarrier is coated with dextran. In some embodiments, the microcarrier is coated with a peptide.

In some embodiments, the microcarriers may be spherical, smooth, macroporous, rod-shaped, or a combination thereof. In some embodiments, the microcarriers may be coupled with protamine or polylysine. In some embodiments, the microcarrier is spherical. In some embodiments, the s microcarrier is ellipsoidal. In some embodiments, the microcarrier is rod-shaped. In some embodiments, the microcarrier is disc-shaped. In some embodiments, the microcarrier is porous. In some embodiments, the microcarrier is non-porous. In some embodiments, the microcarrier is smooth. In some embodiments, the microcarrier is flat.

In some embodiments, the microcarriers are neutral. In some embodiments, the microcarriers are negatively charged. In some embodiments, the microcarriers are hydrophilic.

In some embodiments, the microcarriers may have a surface area of 25 cm², 50 cm², 75 cm², 100 cm², 125 cm², 150 cm², 175 cm², 200 cm², 225 cm², 250 cm², 500 cm², 625 cm², 750 cm², 1,000 cm², 1,250 cm², 5,000 cm², or 7,500 cm². The surface area may be any value or subrange within the recited ranges, including endpoints.

In specific embodiments, the microcarriers are surface treated to enhance cell attachment, maximizing cell yield and viability. The microcarriers may be comprised of USP Class VI polystyrene material, which provides a consistent platform. In some embodiments, the microcarriers create a synthetic surface on the microcarriers for stem cell expansion. An enhanced attachment surface treatment infuses the surface of the microcarriers with oxygen to improve cell attachment. In some embodiments, the microcarriers are nonpyrogenic. In some embodiments, the microcarriers are optimized for mesenchymal stem cell applications. In specific embodiments, the beads may vary in size from 125-212 μm. In specific embodiments, the density of the microcarriers may be 1.026±0.004. In specific embodiments, the microcarriers may be 360 cm²/gram.

In some embodiments, the method comprises combining the hESCs with laminin or a derivative thereof to improve the cell attachment to the carrier surface. In specific embodiments, the laminin is human laminin 511. As alternative embodiments, several other extracellular matrices may be used for cell attachment, such as including, but not necessarily limited to, vitronectin, fibronectin, collagen, Matrigel@, or derivatives thereof.

In some embodiments, the cells may be cultured for one day, two days, three days, four days, five days, six days, seven days, eight days, nine days, ten days, eleven days, twelve days, thirteen days, or fourteen days.

In some embodiments, the cells may be cultured in a working volume of between 10 nL and 3,000 mL, for example about 10 mL, 20 mL, 30 mL, 40 mL, 50 mL, 100 mL, 250 mL, 500 mL, 750 mL, 1,000 mL, or 3,000 mL. The volume may be any value or subrange within the recited ranges, including endpoints.

In some embodiments, the cultured cells may be expanded further.

In some embodiments, the cultured cells may remain undifferentiated. Undifferentiated cells may be identified by expression of various markers, such as including, but not necessarily limited to, SSEA-5, TRA-1-60, Oct-4, and Nanog. In some embodiments, undifferentiated cells express SSEA-5. In some embodiments, undifferentiated cells express TRA-1-60. In some embodiments, undifferentiated cells express Oct-4. In some embodiments, undifferentiated cells express Nanog. In some embodiments, undifferentiated cells express both SSEA-5 and TRA-1-60. In some embodiments, undifferentiated cells express both Oct-4 and Nanog. In some embodiments, undifferentiated cells express SSEA-5, TRA-1-60, Oct-4, and Nanog (IPC #0).

In some embodiments, the cells may be cultured in a feeder cell-conditioned medium. ES culturing methods may include the use of feeder cell layers which secrete factors needed for stem cell proliferation, while at the same time, inhibiting their differentiation. The culturing is typically effected on a solid surface, for example a surface coated with gelatin or vimentin. Exemplary feeder layers include human embryonic fibroblasts, adult fallopian epithelial cells, primary mouse embryonic fibroblasts (PMEF), mouse embryonic fibroblasts (MEF), murine fetal fibroblasts (MFF), human embryonic fibroblast (HEF), human fibroblasts obtained from the differentiation of human embryonic stem cells, human fetal muscle cells (HFM), human fetal skin cells (HFS), human adult skin cells, human foreskin fibroblasts (HFF), human umbilical cord fibroblasts, human cells obtained from the umbilical cord or placenta, and human marrow stromal cells (hMSCs). Growth factors may be added to the medium to maintain the ESCs in an undifferentiated state. Such growth factors include bFGF and/or TGF. In another embodiment, agents may be added to the medium to maintain the hESCs in a naive undifferentiated state—see for example Kalkan et al., 2014, Phil. Trans. R. Soc. B, 369: 20130540.

hESCs are typically plated on top of the feeder cells 1-4 days later in a supportive medium (e.g. NUTRISTEM®, NUT(+) with human serum albumin, mTeSR™ plus, or mTeSR™1 StemFit®). Additional factors may be added to the medium to prevent differentiation of the ESCs such as bFGF and TGFβ3. Once a sufficient amount of hESCs is obtained, the cells may be mechanically disrupted (e.g. by using a sterile tip or a disposable sterile stem cell tool; 14602 Swemed). Alternatively, the cells may be removed by enzymatic treatment (e.g. collagenase A, or TrypLE™ Select). This process may be repeated several times to reach the necessary amount of hESC. According to some embodiments, following the first round of expansion, the hESCs are removed using TrypLE™ Select and following the second round of expansion, the hESCs are removed using collagenase A.

Feeder cell free systems have also been used in ES cell culturing, such systems utilize matrices supplemented with serum replacement, cytokines and growth factors (including IL6 and soluble IL6 receptor chimera) as a replacement for the feeder cell layer. Stem cells can be grown on a solid surface such as an extracellular matrix (e.g., MATRIGEL®, laminin or vitronectin) in the presence of a culture medium—for example the Lonza L7™ system, mTeSR™, StemPro™, XFKS®, E8, NUTRISTEM®). Unlike feeder-based cultures which require the simultaneous growth of feeder cells and stem cells and which may result in mixed cell populations, stem cells grown on feeder-free systems are easily separated from the surface. The culture medium used for growing the stem cells contains factors that effectively inhibit differentiation and promote their growth such as MEF-conditioned medium and bFGF.

Also within the scope of the present disclosure are methods for expanding and maintaining human embryonic stem cells (hESCs) in an undifferentiated state, comprising culturing human pluripotent stem cells on a non-adherent surface to obtain a population of undifferentiated hESCs, combining said population of undifferentiated hESCs with microcarriers in growth media, and expanding said population of cells.

Examples of non-adherent cell culture plates include those manufactured by Nunc (e.g. Hydrocell Cat No. 174912), etc. In other embodiments, non-adherent suspension culture dishes may be used (e.g., Corning).

According to some embodiments, when the cells are cultured on the non-adherent substrate, e.g. cell culture plates, the atmospheric oxygen conditions are 20%. However, manipulation of the atmospheric oxygen conditions is also contemplated such that the atmospheric oxygen percent is less than about 20%, 15%, 10%, 9%, 8%, 7%, 6% or even less than about 5% (e.g. between 1%-20%, 1%-10% or 0-5%). According to other embodiments, the cells are cultured on the non-adherent substrate initially under normal atmospheric oxygen conditions and then lowered to less than normal atmospheric oxygen conditions.

While methods described above are directed to methods of expanding and maintaining hESCs, analogous methods directed to induced pluripotent stem cells (iPSCs) are also within the scope of the present disclosure. iPSCs are a type of stem cell derived from somatic cells which have been reprogrammed back into a pluripotent state through the introduction of pluripotency associated genes, and are available from a variety of sources. The person of ordinary skill in the art will appreciate the changes necessary to adapt the hESC methods described above for use with iPSCs and the like.

It is appreciated that commercially available stem cells can also be used in aspects and embodiments of the present disclosure. Human ES cells may be purchased from the NIH human embryonic stem cells registry, www.grants.nih.govstem_cells/or from other hESC registries. Non-limiting examples of commercially available embryonic stem cell lines are HADC102, ESI, BGO 1, BG02, BG03, BG04, CY12, CY30, CY92, CY1O, TE03, TE32, CHB-4, CHB-5, CHB-6, CHB-8, CHB-9, CHB-10, CHB-11, CHB-12, HUES 1, HUES 2, HUES 3, HUES 4, HUES 5, HUES 6, HUES 7, HUES 8, HUES 9, HUES 10, HUES 11, HUES 12, HUES 13, HUES 14, HUES 15, HUES 16, HUES 17, HUES 18, HUES 19, HUES 20, HUES 21, HUES 22, HUES 23, HUES 24, HUES 25, HUES 26, HUES 27, HUES 28, CyT49, RUES3, WA01, UCSF4, NYUES 1, NYUES2, NYUES3, NYUES4, NYUESS, NYUES6, NYUES7, UCLA 1, UCLA 2, UCLA 3, WA077 (H7), WA09 (H9), WA13 (H13), WA14 (H14), HUES 62, HUES, 63, HUES 64, CT1, CT2, CT3, CT4, MA135, Eneavour-2, WIBR 1, WIBR2, WIBR3, WIBR4, WIBRS, WIBR6, HUES 45, Shef3, Shef6, BINhem19, BJNhem20, SAGO 1, and SA001.

### Compositions

The disclosure provides populations of cells expressing the markers described herein. The skilled artisan will appreciate that the populations of cells as described herein can comprise mixed populations of cell types, whose identities are reflected in percentages of cells in the population expressing one or more of the markers described herein. Individual cells in the population may express only a single marker described below, or individual cells may express combinations of markers described below, depending on the differentiation state of the cells.

In some embodiments, cells in the population express one or more of SIX homeobox 3 (six3), SIX homeobox 6 (six6), phosphodiesterase 6H (PDE 6H), visual system homeobox 2 (CHX10 or VSX2), premelanosome protein (PMEL), protein kinase C alpha (PKCa), ELAV like RNA binding protein 3/4 (HuC/D), orthodenticle homeobox 2 (Otx2), neuronal differentiation 1 (NeuroD), B lymphocyte-induced maturation protein-1 (Blimp1). Transducin, Phosducin (PdC), retinoid X receptor gamma (RXRy), thyroid hormone receptor isoform (Tr-32), atonal bHLH transcription factor 7 (Atoh7), insulin gene enhancer protein (Isl-1), retinal homeobox gene 1 (Rx1), paired box 6 (Pax6), LIM homeobox 2 (LHX2), or retina and anterior neural fold homeobox (RAX).

In some embodiments, cells in the population express one or more Early Eye Field markers selected from the group consisting of Six3, Six6, Rx1, Rax, Pax6, RXRy, and Lhx2.

In some embodiments, cells in the population express one or more Embryonic Retina markers selected from the group consisting of Otx2, NeuroD, Blimp1, Transducin, Phosducin (PdC), RXRy and Trf-32, Atoh7 and Isl-1.

In some embodiments, cells in the population express one or more photoreceptor neuronal cell markers selected from the group consisting of Cone-rod homeobox (Crx), Recoverin, Cone arrestin (CAR) and Rhodopsin.

In some embodiments, cells in the population express one or more photoreceptor neuronal cell markers selected from the group consisting of Cone-rod homeobox (Crx), Recoverin, and Cone arrestin (CAR).

In some embodiments, greater than or equal to 1% of cells, greater than or equal to 3% of cells, greater than or equal to 20% of cells, greater than or equal to 50% of cells, greater than or equal to 70% of cells, greater than or equal to 90% of cells, or greater than or equal to 99% of cells in the population express one or more early eye field markers. In some embodiments, between about 1% to about 99%, about 3% to about 90%, about 20% to about 70%, about 5% to about 50%, about 10% to about 50%, or about 10% to about 80% of cells in the population express one or more early eye field markers.

In some embodiments greater than or equal to 1% of cells, greater than or equal to 3% of cells, greater than or equal to 20% of cells, greater than or equal to 50% of cells, greater than or equal to 70% of cells, greater than or equal to 90% of cells, or greater than or equal to 99% of cells in the population express one or more embryonic retina markers. In some embodiments, between about 1% to about 99%, about 3% to about 90%, about 20% to about 70%, about 5% to about 50%, about 10% to about 50%, or about 10% to about 80% of cells in the population express one or more embryonic retina markers.

In some embodiments, greater than or equal to 10% of the cells in the population express Cone-rod homeobox (Crx); greater than or equal to 3% of the cells in the population express Recoverin; greater than or equal to 3% of the cells in the population express Cone arrestin (CAR); and less than or equal to 1% of the cells in the population express TRA-1-60 and/or SSEA5.

In some embodiments, greater than or equal to 50% of the cells in the population express Cone-rod homeobox (Crx); greater than or equal to 12% of the cells in the population express Recoverin; greater than or equal to 60% of the cells in the population express Cone arrestin (CAR); and less than or equal to 1% of the cells in the population express TRA-1-60 and/or SSEA5.

In some embodiments, greater than or equal to 60% of the cells in the population express Cone-rod homeobox (Crx); greater than or equal to 70% of the cells in the population express Recoverin; greater than or equal to 75% of the cells in the population express Cone arrestin (CAR); and less than or equal to 1% of the cells in the population express TRA-1-60 and/or SSEA5.

In some embodiments, between about 45% to about 65% of the cells in the population express Cone-rod homeobox (Crx); between about 10% to about 15% of the cells in the population express Recoverin; between about 50% to about 70% of the cells in the population express Cone arrestin (CAR); and between about 0.001% to about 1.5% of the cells in the population express TRA-1-60 and/or SSEA5.

In some embodiments, between about 50% to about 70% of the cells in the population express Cone-rod homeobox (Crx); between about 60% to about 80% of cells in the population express Recoverin; between about 70% to about 80% of cells in the population express Cone arrestin (CAR); and between about 0.001% to about 1.5% of the cells in the population express TRA-1-60 and/or SSEA5.

In some embodiments, greater than or equal to 5% of cells, greater than or equal to 10% of cells, greater than or equal to 50% of cells, greater than or equal to 70% of cells, or greater than 90% of cells in the population express Crx. In some embodiments greater than or equal to 10% of cells in the population express Crx. In some embodiments greater than or equal to 15% of cells in the population express Crx. In some embodiments, between about 5% to about 90% of cells, about 10% to about 70% of cells, or about 50% to about 60% of cells in the population express Crx. In some embodiments, between about 40% to about 60% of the cells express Crx. In some embodiments, between about 60% to about 80% of the cells express Crx.

In some embodiments, greater than or equal to 1% of cells, greater than or equal to 3% of cells, greater than or equal to 10% of cells, greater than or equal to 20% of cells, greater than or equal to 50% of cells, greater than or equal to 70% of cells, greater than or equal to 90% of cells, or greater than or equal to 99% of cells in the population express Recoverin. In some embodiments, greater than or equal to 3% of cells in the population express Recoverin. In some embodiments, greater than or equal to 5% of cells in the population express Recoverin. In some embodiments, between about 1% to about 99%, about 3% to about 90%, about 20% to about 70%, or about 50% to about 60% of cells in the population express Recoverin. In some embodiments, between about 10% to about 15% of the cells express Recoverin. In some embodiments, between about 65% to about 85% of cells express Recoverin.

In some embodiments, greater than or equal to 3% of cells, greater than or equal to 5% of cells, greater than or equal to 20% of cells, greater than or equal to 50% of cells, greater than or equal to 70% of cells, greater than or equal to 90% of cells, or greater than or equal to 99% of cells in the population express CAR. In some embodiments, greater than or equal to 3% of cells in the population express CAR. In some embodiments, greater than or equal to 5% of cells in the population express CAR. In some embodiments, greater than or equal to 6% of cells in the population express CAR. In some embodiments, greater than or equal to 10% of cells in the population express CAR. In some embodiments, between about 3% to about 99%, about 5% to about 90%, about 20% to about 70%, or about 50% to about 60% of cells in the population express CAR. In some embodiments, about 50% to about 70% of cells in the population express CAR. In some embodiments, about 65% of cells to about 75% of cells in the population express CAR. In some embodiments bout 70% of cells to about 80% of cells in the population express CAR.

In some embodiments, greater than or equal to 3% of cells, greater than or equal to 5% of cells, greater than or equal to 20% of cells, greater than or equal to 50% of cells, greater than or equal to 70% of cells, greater than or equal to 90% of cells, or greater than or equal to 99% of cells in the population express Rhodopsoin. In some embodiments, greater than or equal to 3% of cells in the population express Rhodopsin. In some embodiments, greater than or equal to 5% of cells in the population express Rhodopsin. In some embodiments, greater than or equal to 6% of cells in the population express Rhodopsin. In some embodiments, greater than or equal to 10% of cells in the population express Rhodopsin. In some embodiments, between about 3% to about 99%, about 5% to about 90%, about 20% to about 70%, or about 50% to about 60% of cells in the population express Rhodopsin. In some embodiments, about 50% to about 70% of cells in the population express Rhodopsin. In some embodiments, about 65% of cells to about 75% of cells in the population express Rhodopsin. In some embodiments bout 70% of cells to about 80% of cells in the population express Rhodopsin.

In some embodiments, less than or equal to 0.001%, less than or equal to 0.01%, less than or equal to 0.1%, less than or equal to 1%, or less than or equal to 5% of cells in the population express TRA-1-60. In some embodiments, less than or equal to 0.1% of cells in the population express TRA-1-60. In some embodiments, 0 to about 5%, about 0.001% to about 1%, or about 0.01% to about 0.1% of cells in the population express TRA-1-60.

In some embodiments, less than or equal to 0.001%, less than or equal to 0.01%, less than or equal to 0.1%, less than or equal to 1%, or less than or equal to 5% of cells in the population express SSEA5. In some embodiments, less than or equal to about 0.1% of cells in the population express SSEA5. In some embodiments 0 to about 5%, about 0.001 to about 1%, or about 0.01 to about 0.1% of cells in the population express SSEA5.

In some embodiments, greater than or equal to 10%, greater than or equal to 20%, greater than or equal to 30%, or greater than or equal to 40% of the cells in the population express Pax6. In some embodiments, between about 3% to about 99%, about 5% to about 90%, about 20% to about 70%, or about 50% to about 60% of cells in the population express Pax6.

In some embodiments, less than or equal to 50%, less than or equal to 40%, less than or equal to 30%, less than or equal to 20%, or less than or equal to 10% of the cells in the population express beta tubulin 3. In some embodiments, less than or equal to 40% of the cells in the population express beta tubulin 3.

In some embodiments, less than or equal to 50%, less than or equal to 40%, less than or equal to 30%, less than or equal to 20%, or less than or equal to 10% of the cells in the population express premelanosome protein (PMEL). In some embodiments, less than or equal to 30% of the cells in the population express PMEL.

In some embodiments, less than or equal to 50%, less than or equal to 40%, less than or equal to 30%, less than or equal to 20%, or less than or equal to 10% of the cells in the population express PKCa. In some embodiments, less than or equal to 30% of the cells in the population express PKCa.

In some embodiments, less than or equal to 20%, less than or equal to 15%, less than or equal to 10%, less than or equal to 5%, or less than or equal to 3% of the cells in the population express HuCD. In some embodiments, less than or equal to 10% of the cells in the population express HuCD.

In some embodiments, between 30% to 90% of cells in the population express Pax6; between 5% to 30% of cells in the population express beta tubulin 3; between 1% to 30% of cells in the population express PMEL; between 3% to 30% of cells in the population express PKCa, and/or between 0.5% to 10% of cells in the population express HuCD.

Methods of determining marker expression will be known to persons of ordinary skill in the art and include, inter alia, flow cytometry analysis (FACS), and immunohistochemistry based methods.

In some embodiments, the population of cells comprises retinal cells. In some embodiments, the population of retinal cells comprises early eye field cells, embryonic retinal cells, photoreceptor cells which may comprise precursors of photoreceptor neuronal cells (PNCs) or mature photoreceptor neuronal cells, or any combination thereof. In some embodiments the population of retinal cells comprises neuronal retina cells (NRCs), Muller glia cells, retinal pigmented epithelial (RPE) cells, or any combination thereof. In some embodiments, the NRCs comprise PNCs, retinal ganglion cells, horizontal neurons, amacrine neurons, rod bipolar cells, cone bipolar cells, rod photoreceptor cells, cone photoreceptor cells or any combination thereof.

### Pharmaceutical Compositions

The disclosure provides pharmaceutical compositions comprising populations of cells expressing the markers described herein and a pharmaceutically acceptable carrier.

In some embodiments, provided herein are compositions comprising cells and cell populations prepared by the methods described. For example, provided herein are pharmaceutical compositions for administrating to a subject, where the composition includes the mature PNCs prepared according to the methods described herein. For example, the composition can also include a cryopreservation media or medium. In some embodiments, the cryopreservation medium comprises a cryoprotective agent which may comprise one or more of adenosine, dextran-40, lactobionic acid, HEPES (N-(2-Hydroxyethyl) piperazine-N′-(2-ethanesulfonic acid)), sodium hydroxide, L-glutathione, potassium chloride, potassium bicarbonate, potassium phosphate, dextrose, sucrose, mannitol, calcium chloride, magnesium chloride, potassium hydroxide, sodium hydroxide, dimethyl sulfoxide (DMSO), and water.

In embodiments, the pharmaceutical composition includes about 0.1% to about 40% of the cryoprotective agent. In embodiments, the pharmaceutical composition includes about 0.10% to about 30% of the cryoprotective agent. In embodiments, the pharmaceutical composition includes about 0.1% to about 20% of the cryoprotective agent. In embodiments, the pharmaceutical composition includes about 0.1% to about 10% of the cryoprotective agent. In embodiments, the pharmaceutical composition includes about 1% to about 10% of the cryoprotective agent. In embodiments, the pharmaceutical composition includes about 0.1% to about 5% of the cryoprotective agent. In embodiments, the pharmaceutical composition includes about 1% to about 40% of the cryoprotective agent. In embodiments, the cryopreservation medium includes about 1% to about 30% of the cryoprotective agent. In embodiments, the pharmaceutical composition includes about 1% to about 20% of the cryoprotective agent. In embodiments, the pharmaceutical composition includes about 1% to about 10% of the cryoprotective agent. In embodiments, the pharmaceutical composition includes about 1% to about 5% of the cryoprotective agent. The percentage may be measured as weight of cryoprotective agent per volume of medium. The percentage may be measured as volume of cryoprotective agent per volume of medium. The percentage may be any value or subrange within the recited ranges, including endpoints.

In some embodiments, the pharmaceutical composition comprises at least about 1,000 cells, at least about 5,000 cells, at least about 25,000 cells, 50,000 cells, at least about 250,0000 cells, at least about 500,000 cells, at least about 1 million cells, at least about 10 million cells, at least about 20 million cells, or at least about 25 million cells. In some embodiments the pharmaceutical composition comprises between about 5,000 cells and 25 million cells, between about 50,000 cells and 10 million cells, between about 100,000 cells and 1 million cells, between about 500,000 cells and 10 million cells, between about 1 million cells and 10 million cells, or between about 1 million cells and 5 million cells. In some embodiments the pharmaceutical composition comprises between about 5,000 cells and 25 million cells. In some embodiments the pharmaceutical composition comprises between about 50,000 cells and 10 million cells. In some embodiments the pharmaceutical composition comprises between about 100,000 cells and 25 million cells.

In embodiments, the pharmaceutical composition includes populations of cells as described herein or populations of cells prepared according to the methods herein and the cells can be at a concentration from about 1×10⁵ cells per mL to about 100×10⁶ cells per mL, or from about 1×10⁵ cells per mL to about 1×10⁶ cells per mL. In some embodiments, the pharmaceutical composition comprises from about 100,000 cells per mL to about 100 million cells per mL, from about 250,000 cells to about 50 million cells per mL, from about 500,000 cells to about 25 million cells per mL, or from about 2 million to about 5 million cells per mL. In some embodiments, the pharmaceutical compositions comprise from about 2×10⁶ cells per mL to about 5×10⁶ cells per mL. In some embodiments, the pharmaceutical composition comprises between about 100,000 cells and 1 million cells. In some embodiments, the pharmaceutical composition comprises between about 100,000 cells and 500,000 cells. In some embodiments, the pharmaceutical composition comprises between about 500,000 cells and 1 million cells.

In other embodiments, the pharmaceutical composition including the cells prepared by the methods herein can be stored in a volume from about 100 μI, to about 1 mL, or about 250 μL, or about 600 μL, or about 1000 mL. The pharmaceutical composition can further include a cryopreservation media, wherein the cryopreservation media includes a cryosolution, e.g. CryoStor® (CS2, CS5 or CS10) or CryoStem™.

In some embodiments, the present disclosure relates to a method of making a pharmaceutical composition comprising a population of cells as described herein. In embodiments, the pharmaceutical composition includes cells prepared according to the methods herein and the cells (e.g., PNCs or mature PNCs) express one or more markers selected from Crx, Recoverin, Cone Arrestin (CAR), or combinations thereof. The pharmaceutical composition may also include other NRCs, e.g. RPEs. In some embodiments, the method of making a pharmaceutical composition comprising a population of retinal cells derived herein comprises culturing a population of undifferentiated pluripotent stem cells under culture conditions sufficient to differentiate cells in the population of the undifferentiated cells into retinal cells; and produce a cellular aggregate, wherein the cellular aggregate is between 100 μm-800 μm in diameter.

In some embodiments, the pharmaceutical composition comprises a population of cells in a suspension, e.g. suspended in a pharmaceutically acceptable carrier, diluent or excipient.

In embodiments, the pharmaceutical compositions can be formulated for administration to the eye by injection, for example, by subretinal, intravitreal or suprachoroidal injection. Formulations for injection may be presented in unit dosage form, for example, in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, for example, sterile pyrogen-free water, before use. In addition to the formulations described previously, the compositions can also be formulated as a depot preparation. Such long-acting formulations can be administered by implantation (e.g., subcutaneously). Thus, for example, the compositions can be formulated with suitable polymeric or hydrophobic materials (for example as an emulsion in an acceptable oil) or ion exchange resins, or as sparingly soluble derivatives, for example, as a sparingly soluble salt.

The nature of pharmaceutical compositions as described herein is dependent on the mode of administration and can readily be determined by one of ordinary skill in the art. The pharmaceutical compositions described herein can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, and glycerol. A modulatory compound can be formulated in various ways, according to the corresponding route of administration. For example, liquid solutions can be made for administration by drops into the ear, for injection, or for ingestion; gels or powders can be made for ingestion or topical application. Methods for making such formulations are well known and can be found in, for example, “Remington's Pharmaceutical Sciences.”

Pharmaceutical compositions in accordance with the present disclosure can further comprise a pharmaceutically-acceptable carrier. In embodiments, a composition can be formulated to thaw and administered directly into a subject, e.g. via injection, without additional manipulation prior to administration.

### Scaffolds

In some embodiments of the pharmaceutical compositions of the disclosure, and methods of using same, the population of cells are contained within, and/or disposed upon a scaffold.

Scaffolds can take any suitable form for implantation into the retina, for example a sheet, disk, a capsule, a mesh, or other two and/or three-dimensional structures designed to induce engraftment of the population of cells in appropriate positions in the retina. Without wishing to be bound by theory, it is thought that use of a scaffold can effectively target populations of cells to regions of the retinal that have undergone degeneration or damage, and promote engraftment of the population of cells, and connectivity of the engrafted cells to host retinal cells. As a further advantage, scaffolds can be used to deliver cell populations to large areas of the retina, for example an area that has undergone physical damage (e.g., a tear) or degeneration. For example, two dimensional scaffolds can be used to create planar sheets of retinal progenitor cells and/or retinal cells, which can then be implanted into the subretinal space (Singh, D. et al. Biomaterials 154: 158-168 (2018). By fine tuning the topographical properties of the scaffold, and including micro- or nano-patterned structures on the scaffold with extracellular matrix (ECM) properties, scaffolds can be designed to hold stem cell, retinal progenitor cell, and/or retinal cell populations effectively, and deliver these cell populations as a patch into the subretinal space (Nair et al., Appl Sci (Basel) 11:2154 (2021)). In general, a suitable two-dimensional scaffold should be thin enough to allow nutrient exchange between the choriocapillaris and the retina, and should not physically distort the retina's photoreceptor layer.

In some embodiments, the scaffold is biocompatible. In some embodiments, the scaffold is biodegradable. In some embodiments, the scaffold is biocompatible and biodegradable. Suitable scaffold materials include, but are not limited to materials designed to mimic naturally occurring components of the retinal extracellular matrix, such as collagen, gelatin, chondroitin sulfate, and hyaluronic acid (GCH), and combinations thereof. Such materials are generally non-immunogenic, and promote cell attachment. Additional suitable scaffold materials include, but are not limited to, biocompatible materials such as polyphosphazenes, polyanhydrides, polyacetals, polyorthoesters, polyphosphates, polycaprolactones, polyurethanes, polypeptides, polycarbonates, polyamides, polysaccharides, polyamino acids, other polymers, proteins, metals or ceramics. In some aspects, the scaffold can be formed in whole or in part from a biodegradable hydrogel, which can be hyaluronan-based (e.g., Hystem®). Such hydrogels are described in WO2019/028088, the contents of which are incorporated by reference herein. In addition, hyaluronan-based hydrogels (trade name) that mimic the natural extracellular matrix Environment (ECM, Renevia®) may be suitable for use in the scaffolds described herein. As a further example, the scaffold may comprise decellularized tissue, such as retinal tissue.

Methods of making scaffolds will be known to persons of ordinary skill in the art, and include, inter alia, 3D printing using suitable materials. Alternatively, or in addition, the material of the scaffold can be electrospun, deposited, coated, lyophilized, or crosslinked.

### Methods of Treatment

The disclosure provides methods for treating vision conditions (e.g., eye related diseases resulting in vision loss) in a subject, the methods comprising administering to a subject a therapeutically effective amount of the pharmaceutical compositions described herein.

The disclosure provides method of preventing or treating eye related diseases (e.g., those resulting in vision loss) in a subject in need thereof. In further embodiments, the method comprises administering to the subject an effective amount of a composition comprising the cells prepared in accordance with the methods described herein.

The disclosure provides compositions for use in the treatment or prevention of vision conditions in a subject, the use comprising administering to a subject a therapeutically effective amount of the pharmaceutical compositions described herein.

The disclosure provides compositions for use in the manufacture of a medicament for the treatment of a vision condition in a subject, comprising administering to a subject a therapeutically effective amount of the pharmaceutical compositions described herein.

In some embodiments, the methods for treating eye related diseases (e.g., vision loss) comprises administering to a subject a composition comprising the cells (e.g., PNCs, mature PNCs, or NRCs) prepared according to the methods described herein, in combination with methods for controlling the outset of symptoms. In particular, the combination treatment can include administering readily known treatments which will be known to persons of skill in the art.

In some embodiments, the methods comprise administering a therapeutically effective amount of a pharmaceutical composition comprising a population of retinal cells produced by the methods described herein. In some embodiments, the population of retinal cells comprises a population of cells wherein (a) greater than or equal to 10% of the cells in the population express Cone-rod homeobox (Crx); (b) greater than or equal to 3% of the cells in the population express Recoverin; (c) greater than or equal to 3% of the cells in the population express Cone arrestin (CAR); and (d) less than or equal to 1% of the cells in the population express TRA-1-60 and/or SSEA5. In some embodiments, the population of retinal comprises a population of cells wherein (a) between about 10% to 70% of the cells in the population express Crx; (b) between about 3% to 90% of the cells in the population express Recoverin; (c) between about 3% to 90% of the cells in the population express CAR; and (d) between 0 to about 0.1% of the cells in the population express TRA-1-60 and/or SSEA5. In some embodiments, the population of retinal cells comprises early eye field cells, embryonic retinal cells, precursors of photoreceptor neuronal cells (PNCs), mature photoreceptor neuronal cells, or any combination thereof. In some embodiments, the population of retinal cells comprises neuronal retina cells (NRCs), Muller glia cells, retinal pigmented epithelial (RPE) cells, or any combination thereof. In some embodiments, the NRCs comprise precursors of PNCs, retinal ganglion cells, horizontal neurons, amacrine neurons, rod bipolar cells, cone bipolar cells, rod photoreceptor neuronal cells, cone photoreceptor neuronal cells or any combination thereof. In some embodiments, the population of retinal cells comprises neuronal retina cells (NRCs). In some embodiments, the population of retinal cells comprises photoreceptor cells (e.g., rod and/or cone photoreceptor cells). In some embodiments, the population of retinal cells comprises precursors of PNCs and mature photoreceptor neuronal cells.

In some embodiments, the vision condition comprises (is the result of) a neurodegenerative disease of the retina or damage to the retina.

In some embodiments, the neurodegenerative disease of the retina comprises a genetic disease, for example Stargardt's disease, Leber congenital amaurosis, retinitis pigmentosa, congenital color blindness, Best's disease. Alternatively, or in addition, the neurodegenerative disease of the retina can be caused by an additional underlying pathology (e.g., diabetic retinopathy), and/or environmental factors such as diet, age (age related macular degeneration), sunlight exposure, alcohol exposure and the like.

In some embodiments, the neurodegenerative disease of the retina comprises Leber congenital amaurosis, congenital color vision anomalies, Stargardt's disease, Best's disease, Doyne's disease, cone dystrophy, retinitis pigmentosa, X-linked retinoschisis, Usher syndrome, age-related macular degeneration, atrophic age-related macular degeneration, neovascular AMD, diabetic maculopathy, proliferative diabetic retinopathy (PDR), cystoid macular edema, central serous retinopathy, retinal detachment, intraocular inflammation, glaucoma or posterior uveitis. Neurodegenerative diseases of the retina are described in US20210189430, the contents of which are incorporated by reference. As a further example, neurodegenerative diseases of the retina that can be treated by the compositions and methods described herein include, but are not limited to, Stargardt's disease, diabetic retinopathy, macular degeneration, retinitis pigmentosa, Leber congenital amaurosis, cone-rod dystrophy, choroideremia, and X-linked retinoschisis. In some embodiments, the macular degeneration comprises age related macular degeneration.

In some embodiments, the vision condition is a result of damage to the retina. Damage to the retina can be the result of physical trauma. Alternatively, or in addition, damage to the retina encompasses, but is not limited to, retinal tears, retinal detachment, retinal puckers, epiretinal membrane and macular holes.

The methods and compositions described herein can be used to treat conditions of the macula. The macula is a small area in the center of the retina that provides greater visual acuity for straight-ahead vision. Exemplary macular conditions include, but are not limited to, branch retinal vein occlusion, central retinal vein occlusion, central serous retinopathy, choroidal neovascular membranes, cytomegalovirus retinitis, diabetic retinopathy, histoplasmosis, macular degeneration (dry and wet forms), macular edema, macular hole, macular pucker, macular telangiectasia, retinal detachment, retinitis pigmentosa, retinoblastoma, retinopathy of prematurity, Stargardt disease and Usher syndrome. The skilled artisan will appreciate that the conditions described herein may affect the macula only, or may affect the macula and regions of the retina other than the macula.

In some embodiments, the methods described herein comprise administering the compositions described herein to the eye of the subject. In some embodiments, the compositions are administered into or adjacent to the retina of the subject. For example, the compositions may be administered directly into the retinal layer. Alternatively, or in addition, the compositions may be administered adjacent to the retina, for example into the subretinal space.

In some embodiments, the compositions described herein are administered by injection, implantation, or transplantation.

The described compositions can be administered as a pharmaceutically or physiologically acceptable preparation or composition containing a physiologically acceptable carrier, excipient, or diluent, and administered to the tissues of the recipient organism of interest, including humans and non-human animals. In some embodiments, the compositions comprise a cell suspension, i.e. a population cells generated by the methods described herein suspended in a pharmaceutically acceptable carrier.

In alternative embodiments, the composition comprises a scaffold comprising the population of cells, which is administered to the subject by implantation of transplantation the scaffold comprising the population of cells. In some embodiments, the scaffold comprises a sheet, a disk, a capsule, or a mesh. In some embodiments, the scaffold is biocompatible, biodegradable, or both.

In some embodiments, for example those embodiments where the composition comprises a cell suspension, the composition is administered to the retina via injection. In some embodiments, the injection comprises intravitreal, subretinal or suprachoroidal injection of a suspension comprising the population of cells. Suprachoroidal injection will be known to persons of skill in the art, and is described, for example in U.S. Pat. No. 9,956,114, as well as U.S. Application No. US 2022-0280341, the contents of which are incorporated by reference herein. Subretinal injection and intravitreal injection methods are described, for example in U.S. Pat. Nos. 9,795,452; 9,567,376; 9,433,688, and 10,081,659; as well as U.S. Application No. 2022-0233768, the contents of which are incorporated by reference herein.

In some embodiments, the methods comprise administering volume of the composition that is between about 0.1 μL to about 1 mL, including all numbers within the range, depending on the size of the area to be treated, the concentration of cells, the route of administration, and the desired effect of the method. In some embodiments, the volume is about 50 μL. In some embodiments, the volume is about 70 μL. In some embodiments, the volume is about 100 μL. In some embodiments, the volume is about 125 μL. In some embodiments, the volume is about 150 μL. In some embodiments, the volume is about 175 μL. In some embodiments, the volume is about 200 μL. In some embodiments, the volume is about 250 μL. In some embodiments, the volume is about 300 μL. In some embodiments, the volume is about 450 μL. In some embodiments, the volume is about 500 μL. In some embodiments, the volume is about 600 μL. In some embodiments, the volume is about 750 μL In some embodiments, the volume is about 850 μL. In some embodiments, the volume is about 1000 μL. In some embodiments, the volume is between about 10 μL to about 800 μL, between about 30 μL to 700 μL, between about 50 μL to about 500 μL, between about 50 μL to about 300 μL, between about 50 μL to about 250 μL, between about 100 μL to about 500 μL, between about 100 to about 250 μL, between about 100 μL to about 800 μL, between about 200 μL to about 1 mL, between about 300 μL to about 800 μL, between about 400 μL to about 800 μL, or between about 500 to about 1 mL.

In some embodiments, for example those embodiments where the composition comprises a cell suspension, the population of cells are administered at a concentration of between about 1×10⁴ cells per mL to about 100×10⁷ cells per mL. In some embodiments, the population of cells are at concentration of between about 1×10⁵ cells per mL to about 100×10⁶ cells per mL, or from about 1×10⁵ cells per mL to about 1×10⁶ cells per mL. In some embodiments, the population of cells are at a concentration of between about 1×10⁵ cells per mL to about 100×10⁶ cells per mL.

In some embodiments, the methods comprise thawing composition of a population of cells in a cryopreservation medium described herein, and administering the thawed composition directly to the subject. In alternative embodiments, the methods comprise thawing a population of cells in a cryopreservation medium, followed by culturing the cells and/or transferring the cells to a pharmaceutically acceptable carrier, and administering the cells to the subject.

In embodiments, a therapeutically effective amount of the composition (e.g., the cells (e.g., PNCs, mature PNCs, or NRCs)) in humans can be any therapeutically effective amount. In one embodiment, the composition is administered thrice daily, twice daily, once daily, fourteen days on (four times daily, thrice daily or twice daily, or once daily) and 7 days off in a 3-week cycle, up to five or seven days on (four times daily, thrice daily or twice daily, or once daily) and 14-16 days off in 3 week cycle, or once every two days, or once a week, or once every 2 weeks, or once every 3 weeks. In examples, the composition is administered once a week, or once every two weeks, or once every 3 weeks or once every 4 weeks for at least 1 week, in some embodiments for 1 to 4 weeks, from 2 to 6 weeks, from 2 to 8 weeks, from 2 to 10 weeks, or from 2 to 12 weeks, 2 to 16 weeks, or longer (e.g., 1, 2, 3, 4, 5, 6, 7, 8, 9, 10, 11, 12, 36, 48, or more weeks). In some embodiments, the therapeutically effective amount of the composition is administered every month, every other month, every three months, every four months, every six months, or annual. In some embodiments, a single administration of the composition is sufficient to have a therapeutic effect.

In some embodiments, administration of the compositions described herein reduces the severity of or eliminates one or more clinical signs of a vision condition in the subject. Clinical signs of vision conditions include, but are not limited to, decreased peripheral vision, decreased central vision (e.g., when reading), decreased night vision, loss of color perception, reduction in visual acuity, decreased photoreceptor function, and pigmentary changes.

In some embodiments, administration of the compositions described herein reduces the severity of or eliminates retinal degeneration or damage. For example, administration of the compositions described herein can reduce the degenerated or damage area of the retina, reduce the severity of degeneration or damage, or reduce the number of damaged, dead or dying cells. Methods of measuring retinal damage will be apparent to the skilled artisan and include, but are not limited to, optical coherence tomography (OCT), electroretinography (ERG), and fundus autofluorescence imaging.

### Kits and Articles of Manufacture

The disclosure provides kits comprising the populations of cells, or pharmaceutical compositions comprising same, produced using the methods described herein. In some embodiments, the population of cells comprises NRCs, mature PNCs, and/or PNCs prepared in accordance with the methods described herein. In some embodiments, the kit further comprises reagents (e.g., cryopreservation reagents and pharmaceutically acceptable carriers or diluents).

The present invention also provides packaging and kits comprising pharmaceutical compositions for use in the methods of the present invention. The kit can comprise one or more containers selected from the group consisting of a bottle, a vial, an ampoule, a blister pack, syringe and cryovial. The kit can further include one or more of instructions for use in treating and/or preventing a disease, condition or disorder of the present invention (e.g., an eye related disease, e.g., vision loss), one or more syringes, one or more applicators, or a sterile solution suitable for thawing and reconstituting a pharmaceutical composition of the present invention.

### Exemplary Embodiments

The invention can be understood with reference to the following enumerated embodiments.

Embodiment 1. A method for obtaining a population of neural retina cells (NRCs) from undifferentiated pluripotent stem cells, the method comprising:
(a) growing the undifferentiated pluripotent stem cells in a dynamic culture;
(b) seeding the undifferentiated pluripotent stem cells as single cells in a culture vessel;
   and
(c) differentiating the undifferentiated pluripotent stem cells in the culture vessel under conditions to obtain the population of NRCs.

Embodiment 2. The method of embodiment 1, wherein the culture vessel is a bioreactor.

Embodiment 3. The method of embodiment 2, wherein the bioreactor is a vertical wheel bioreactor.

Embodiment 4. The method of embodiments 1-3, wherein the undifferentiated pluripotent stem cells are seeded for differentiation as single cells and grown in suspension as cell aggregates for at least 14-18 weeks.

Embodiment 5. The method of embodiment 4, wherein the cell aggregates have a size controlled by a rotation velocity of the culture vessel, wherein the size of the aggregates is from about 100 μm to about 800 μm.

Embodiment 6. The method of embodiment 5, wherein the rotation velocity is increased during the differentiation process.

Embodiment 7. The method of any one of embodiments 1 to 6, wherein step (c) comprises:
(i) culturing the undifferentiated pluripotent stem cells, for a first time period under culture conditions sufficient to induce the stem cells to form cell aggregates, wherein the cell aggregates comprise cells expressing one or more Early Eye Field markers selected from Rx1, Pax6, RXRy, LHX2 and RAX; and
(ii) culturing the cell aggregates from step i) for a second time period under culture conditions sufficient to differentiate the cell aggregates to cells expressing one or more Embryonic Retina markers selected from Otx2, NeuroD, Blimp1, Transducin, Phosducin (PdC), RXRy and Tr-32, Atoh7 or IS1-1; and
(iii) culturing the cells expressing the one or more Embryonic Retina markers from step ii) for a third time period under culture conditions sufficient to differentiate the cells to precursors of photoreceptor neural cells (PNCs) and mature PNCs, wherein the PNCs express one or more proteins selected from Crx, Recoverin, or Cone arrestin (CAR).

Embodiment 8. The method of any one of embodiments 1 to 7, wherein the pluripotent stem cells are human embryonic stem cells (hESCs).

Embodiment 9. The method of any one of embodiments 1 to 7, wherein the pluripotent stem cells are human induced pluripotent stem cells (hiPSCs).

Embodiment 10. The method of any one of embodiments 1 to 7, wherein step (c) comprises differentiating for a first time period with a Rock Inhibitor (RI), nicotinamide (NIC), and a Wnt inhibitor, differentiating for a second time period with NIC and IWRe1, differentiating for a third time period with NIC and insulin growth factor-1 (IGF-1), differentiating for a fourth time period with NIC, IGF-1 and a Notch inhibitor, and differentiating for a fifth time period with IGF-1, retinoic acid (RA), taurine and NT4.

Embodiment 11. The method of embodiment 10, wherein the first time period is 0-7 days, and/or the second time period is 2-20 days, and/or the third time period is 10 days to 8 weeks, and/or the fourth time period is 4-10 weeks, and/or the fifth time period is 12-18 weeks.

Embodiment 12. The method of any one of embodiments 1 to 7, wherein the first time period is from about 3 days to about 120 days, or from about 5 days to about 15 days, or about 3 days.

Embodiment 13. The method of embodiment 7, wherein the second time period is from about 1 days to about 120 days, or from about 5 days to about 15 days, or about 11 days.

Embodiment 14. The method of embodiment 7, wherein the cells in step ii) are cryopreserved between days 14 to 120.

Embodiment 15. The method of Embodiment 14, wherein cryopreservation is in a cryosolution.

Embodiment 16. The method of embodiment 7, wherein the cryosolution comprises CryoStem.

Embodiment 17. The method of embodiment 7, further comprising the step of cryopreserving the cells from step (ii) at or about the completion of the first time period.

Embodiment 18. The method of embodiment 16 or 17, wherein the cryopreserved cells are thawed, and wherein the thawed cells are cultured.

Embodiment 19. The method of embodiment 18, wherein the thawed cells are cultured in accordance with embodiment 7 step (iii).

Embodiment 20. The method of embodiment 7, wherein the cells from step iii) are cryopreserved after the third time period.

Embodiment 21. The method of embodiment 20, wherein the cryopreserved cells are thawed and cultured in any remaining steps of the method.

Embodiment 22. A method of preparing a photoreceptor neural cell (PNC) composition for administration to a subject directly after thawing, said method comprising: (a) suspending the PNCs prepared according to any one of embodiments 1 to 21 in a cryopreservation media to form a cell suspension, (b) storing the cell suspension at a cryopreservation temperature, and (c) thawing the cryopreserved suspension.

Embodiment 23. The method of embodiment 22, wherein the cryopreservation media comprises one or more of adenosine, dextran-40, lactobionic acid, HEPES (N-(2-Hydroxyethyl) piperazine-N′-(2-ethanesulfonic acid)), sodium hydroxide, L-glutathione, potassium chloride, potassium bicarbonate, potassium phosphate, dextrose, sucrose, mannitol, calcium chloride, magnesium chloride, potassium hydroxide, sodium hydroxide, dimethyl sulfoxide (DMSO), or water.

Embodiment 24. A pharmaceutical composition for administrating to a subject, said composition comprising the mature PNCs prepared according to any one of embodiments 7 to 23 and a cryopreservation media.

Embodiment 25. The pharmaceutical composition of embodiment 24, wherein the cryopreservation media comprises one or more of adenosine, dextran-40, lactobionic acid, HEPES (N-(2-Hydroxyethyl) piperazine-N′-(2-ethanesulfonic acid)), sodium hydroxide, L-glutathione, potassium chloride, potassium bicarbonate, potassium phosphate, dextrose, sucrose, mannitol, calcium chloride, magnesium chloride, potassium hydroxide, sodium hydroxide, dimethyl sulfoxide (DMSO), and water.

Embodiment 26. The pharmaceutical composition of embodiments 24 or 25, wherein the PNCs are at a concentration of about 1×10⁵ cells per mL to about 100×10⁶ cells per mL.

Embodiment 27. The pharmaceutical composition of any one of embodiments 24 to 26, wherein the pharmaceutical composition is stored at a volume of about 100 μL to about 1 mL, or about 250 μL, or about 600 μL

Embodiment 28. The pharmaceutical composition of any one of embodiments 24 to 27, wherein the cryopreservation media comprises a cryosolution.

Embodiment 29. The pharmaceutical composition of any one of embodiments 24 to 28, wherein the cryosolution comprises CRYOSTOR® CS2, CRYOSTOR® CS5, CRYOSTOR® CS10 or CRYOSTEM™.

Embodiment 30. The pharmaceutical composition of any one of embodiments 22 to 29, wherein the PNCs express one or more markers selected from Crx, Recoverin, Cone Arrestin (CAR), or combinations thereof.

Embodiment 31. The method of any one of embodiments 7 to 21, wherein the cells cultured after step (iii) comprise about 1%-30% retinal epithelial cells.

Embodiment 32. A method for treating vision loss in a subject, the method comprising administering to said subject a therapeutically effective amount of the composition according to any one of embodiments 24 to 30.

Embodiment 33. The method of embodiment 32, wherein the administering comprises administering the composition into or adjacent to the retina of the subject.

Embodiment 34. The method of embodiment 32 or 33, wherein the administering is by injection, implantation, or transplantation.

### EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### ABBREVIATIONS AND DEFINITIONS

RI: Rock Inhibitor
NIC: Nicotinamide
IWRe1: Wnt pathway inhibitor
IGF-1: Insulin-like growth factor 1
DAPT: Notch inhibitor
BDNF: brain-derived neurotrophic factor
NT4: neurotrophin-4

### Example 1—Dynamic Differentiation Protocol: Production Run 44 (PR-44)

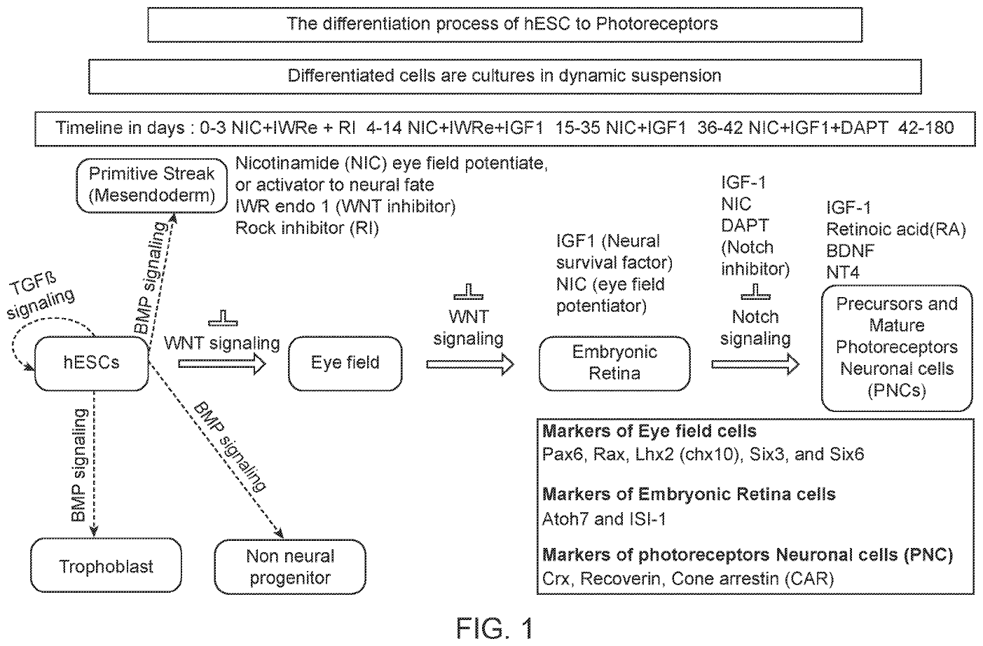

The differentiation of human Embryonic Stem Cells (hESCs) to Retinal cells was based on a stepwise differentiation process in which cells are cultured in dynamic suspension and supplemented with relevant growth factors at each stage (FIG. 1). The process started by seeding hESCs (HADC102) as single cell suspension at two concentrations in a PBS Wheel™ small scale vertical wheel bioreactor.

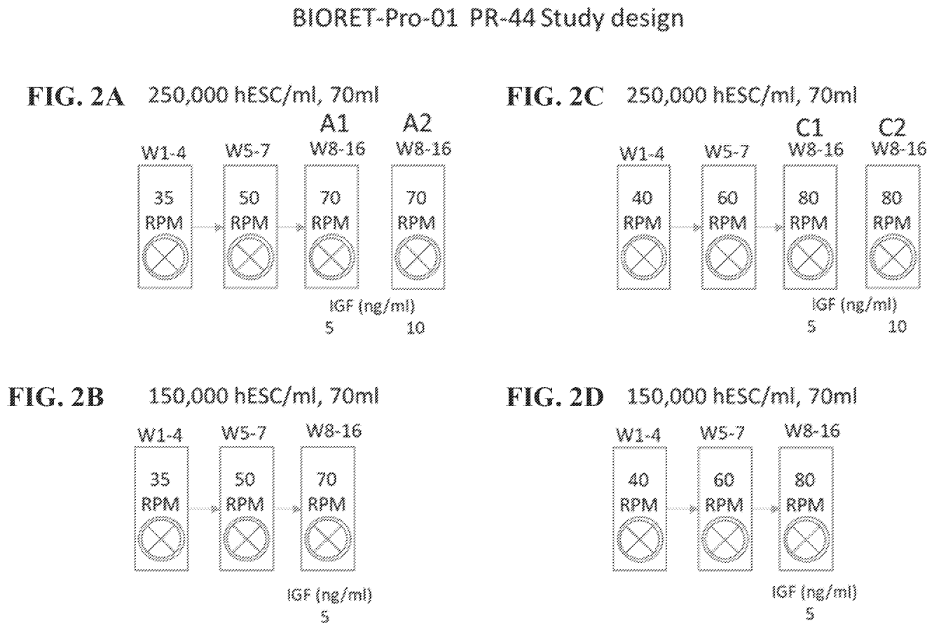

Cells were grown in dynamic culture at four different rotation velocities (A-D) according to FIGS. 2A-2D. The initial PBS Wheel rotation velocity was 35 RPM or 40 RPM, at the time of seeding. The PBS Wheel rotation velocity was increased every 1-3 weeks by 15 RPM or by 20 RPM according to in FIGS. 2A-2D.

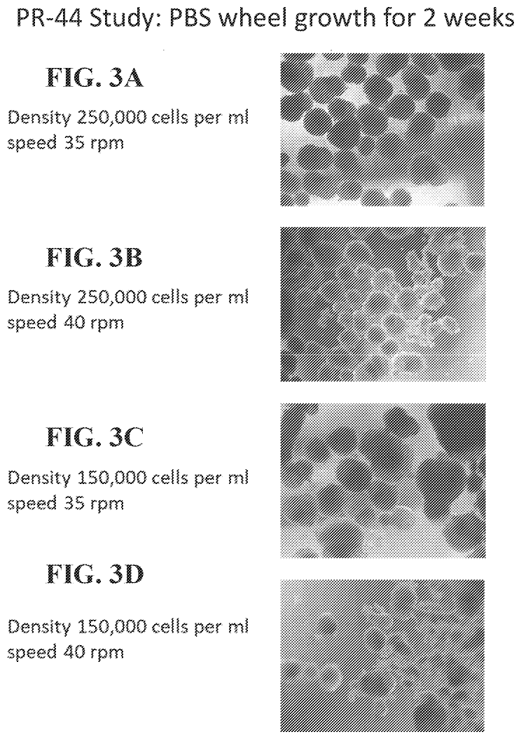

The size of the aggregates was maintained relatively small, between 50 to 100 microns, between 50 to 150 microns, between 100 to 250 microns, or between 150 to 350 microns or between 250 to 450 microns. Aggregates were smaller as the rotation velocity increases (FIGS. 3A-3B). The velocity increased throughout differentiation in order to limit the size of the aggregates, as larger size may correlate with the development of a necrotic core and the center of the aggregate. Variable accessibility of factors into the aggregate may lead to variable cell populations and reduced control on the directed differentiation.

The cells were fed twice per week by removing 75% of the medium and exchanging with fresh media with the factors according to Table 1 below:

TABLE 1
Differentiation reagents list and days of use

| Day in process | Small molecule | Final concentration | |
|---|---|---|---|
| Days 0-3 | RI | 10 | μm |
| | NIC | 10 | mM |
| | IWRel | 3 | μM |
| Days 3-14 | NIC | 10 | mM |
| | IWRe-1 | 3 | μM |
| Weeks 2-5 | NIC | 10 | mM |
| | IGF-1 | 5 | ng/mL |
| Weeks 5-6 | NIC | 10 | mM |
| | IGF-1 | 5 | ng/mL |
| | DAPT | 10 | μM |
| Weeks 7-12/16 | IGF-1 | 5 | ng/mL |
| | Retinoic acid (RA) | 0.5 | μM |
| | Taurine (TA) | 100 | μM |
| | BDNF | 20 | ng/mL |
| | NT4 | 20 | ng/mL |

*RI: Rock Inhibitor; NIC: Nicotinamide, IWRe1: Wnt pathway inhibitor; IGF-1: Insulin-like growth factor 1; DAPT: Notch inhibitor; BDNF: brain-derived neurotrophic factor; NT4: neurotrophin-4.

Differentiation Process of hESCs to Eve Field Cells

At first, the cells were cultured in media containing growth factors including Rock inhibitor (RI), Nicotinamide (NIC), and Wnt inhibitor (IWRe1). This combination of growth factors was used from the initiation of the differentiation to day 3 and was referred to as the first time period.

Then the cells were supplemented with media containing growth factors including NIC and IWRe1. This combination of growth factors was used from day 3 to day 14 and was referred to as the second time period.

Differentiation to Embryonic Retina cells

Differentiation towards Embryonic Retina cells was initiated by culturing the cells with media containing the growth factors NIC, and Insulin growth factor 1 (IGF-1). This combination of growth factors was used from week 2 to week 5 and was referred to as the third time period.

At week #4 of differentiation, the cells were tested for the expression of pluripotent cells using an established hESC residual FCM (flow cytometry) based assay. Results showed that hESC residual population was less than 0.1% (Table 2, below). Cellular differentiation continued for additional 12 weeks.

TABLE 2
Determination of hESCs residual cells
at PR-44 week #6 of differentiation

| QCT-21-076 | % TRA-1-60+ |
|---|---|
| Sample Name | Oct-4+ cells |
| QCFFHESCCN0 l_PC (Positive control) | 86.47% |
| OPRREF01A_NC (Negative control) | 0.00% |
| PR44_Control (static method) | 0.03% |
| PR_A (250,000 hESC/mL) | 0.03% |
| PR_B (150,000 hESC/mL) | 0.10% |

### Differentiation to PNC

Differentiation towards PNCs was initiated by culturing the cells with media containing the growth factors NIC (1-50 mM), IGF-1 (0.5-20 ng/mL), and Notch inhibitor (DAPT 1-50 μM). This combination of growth factors was used from week 5 to week 6 and was referred to as the fourth time period.

Then cells were cultured with media containing growth factors including IGF-1 (0.5-20 ng/mL), Retinoic acid (RA, 0.1-2 μM), Taurine (TA, 10-400 μM), BDNF (2-200 ng/mL), and NT4 (2-200 ng/mL). This combination of growth factors was used from week 7 to either week 12 or week 16, and was referred to as the fifth period of time.

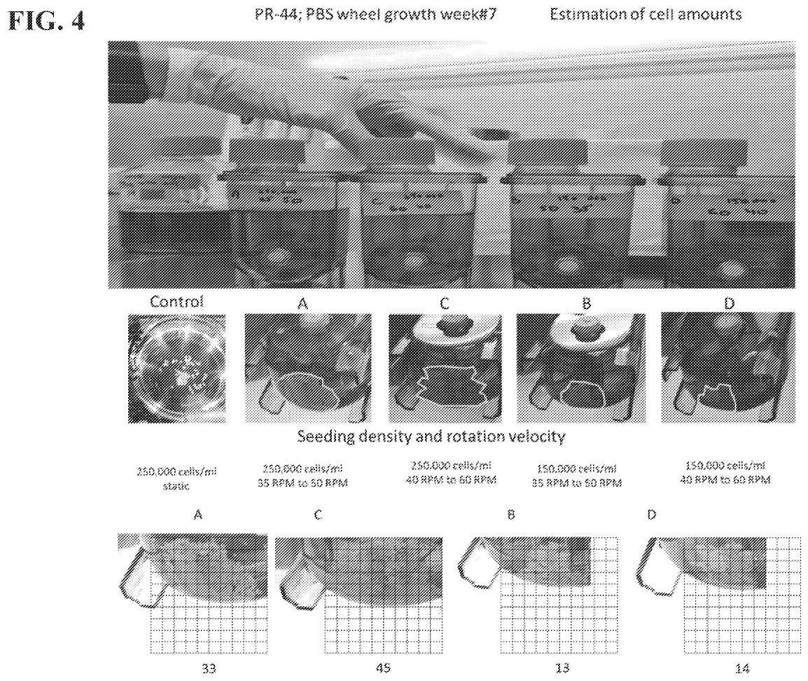

At week #7, the amount of cells was estimated by visual inspection. The 0.1 L bioreactors were removed from their magnetic drive and aggregates were settled at the bottom of the vessels. The area covered by aggregates was assessed to provide an indication of the relative amount of cells in each vessel (FIG. 4). The results showed that the initial seeding densities were the major factor determining the amount of cells 7 weeks into the differentiation process (about twice the amount of cells in the higher seeding density versus the lower seeding density). An additional 35% increase in aggregate amount may be attributed to higher rotational velocity of the dynamic culture (as in Group C Vs. Group A in FIG. 4).

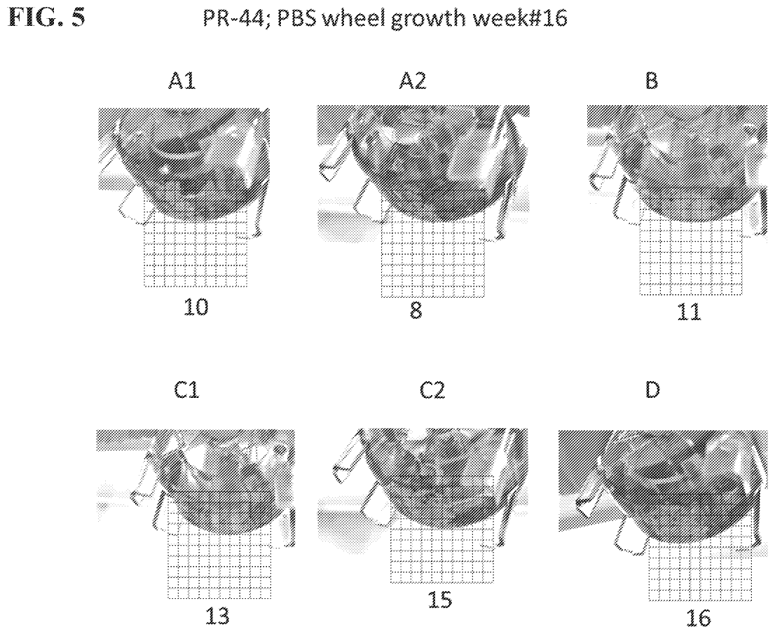

At week #8 cells of groups A and C were roughly split into two. IGF-1 was added at a basic concentration of 5 ng/mL (A1, A2 of FIG. 5) or twice as much at a concentration of 10 ng/mL (C1, C2 of FIG. 5). At week #116, the cell amount was estimated again. As illustrated by FIG. 5, although a significant number of cells were sampled at weeks 4, 8, and 12 for flow cytometry, immunostaining, and QPCR, mainly from groups A1, A2, C1 and C2, there was a higher number of cells in these conditions relative to the corresponding groups B and D, with a total of more than a 10 fold increase in the final product yield relative to the static non adherent culture.

### Example 2—Dynamic Differentiation Protocol: Production Run 45 (PR-45)

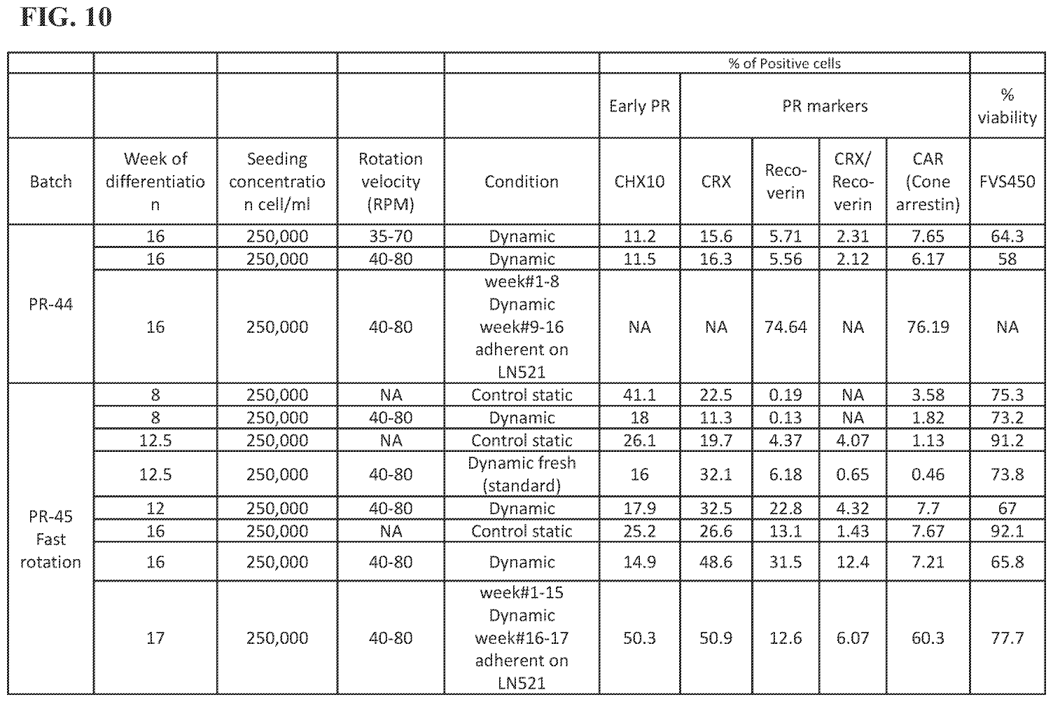

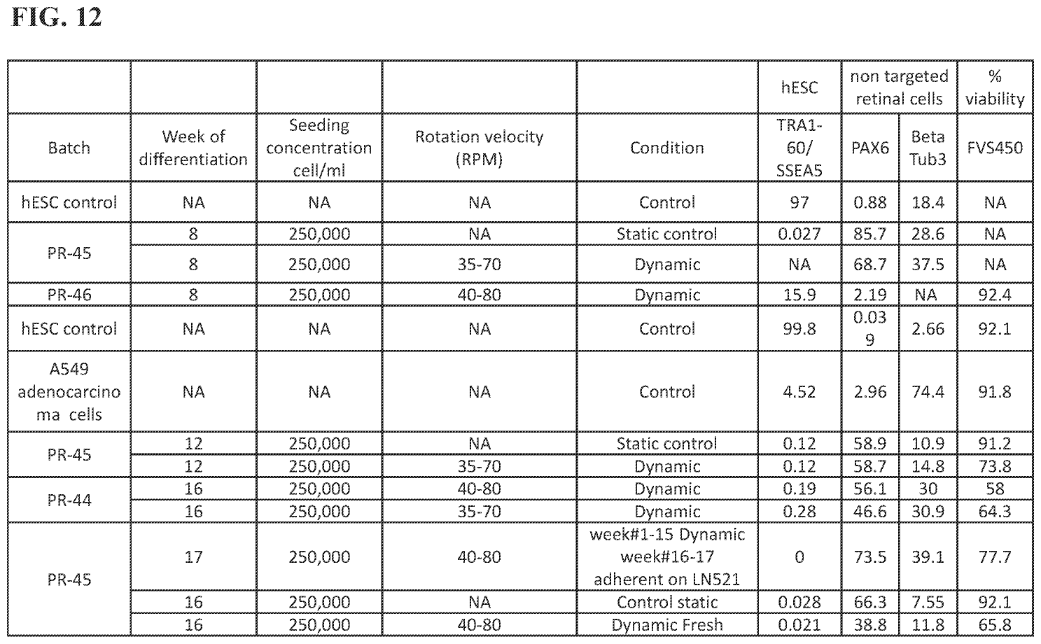

In a repeat of the differentiation process, PR-45, cells were cultured as in Example 1. However the cells were all seeded at the high cell concentration of 250,000/ml and a high rotational velocity of 40-80 RPM (As for PR-44 group C1) was applied for the full 16 weeks of differentiation in several PBS wheel vessels. Some vessels were sampled at the end of 8, 12 and 16 differentiation weeks for the expression of various cell markers using flow cytometry, in comparison to the control static non-adherent cultures that were grown in parallel (FIGS. 10-12). The quantitative monitoring of PR markers expression (FIG. 10) enables in process control that is crucial for achieving viable industrial process, with go-no go point alone the 16 weeks of differentiation.

### Example 3—Quality Determination: Production Run 46 (PR-46)

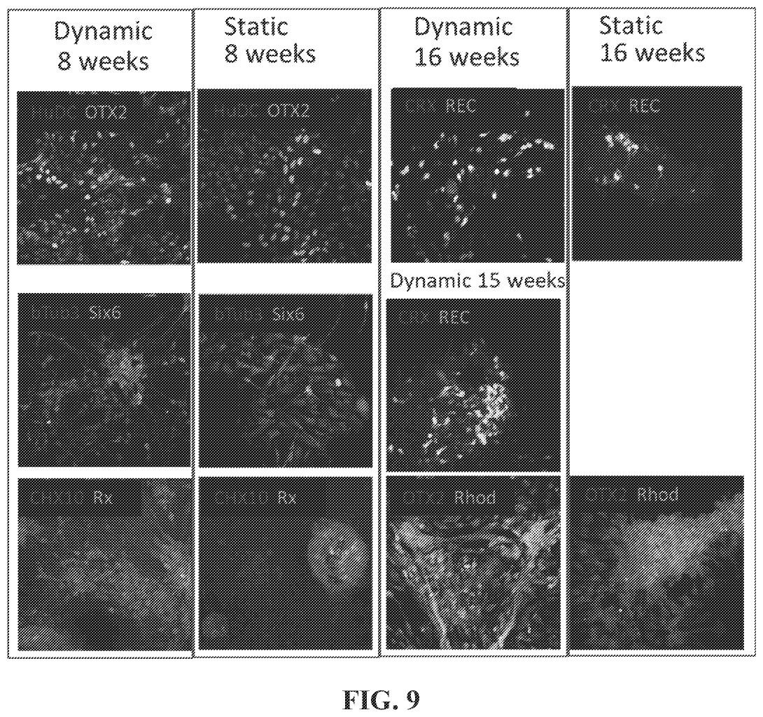

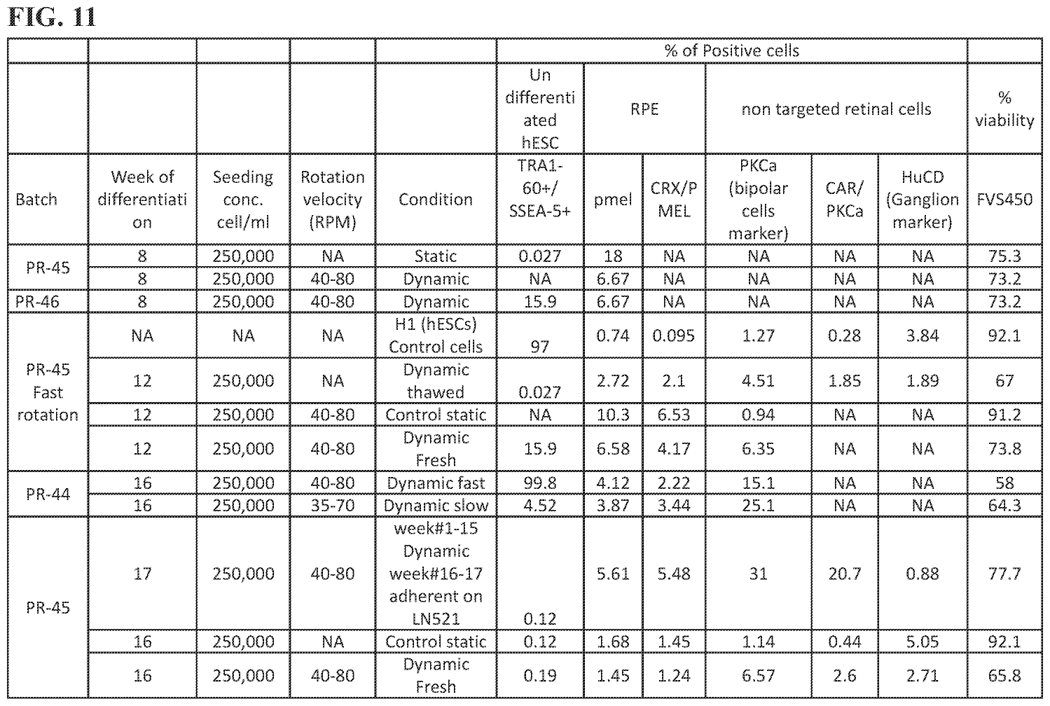

Beside the PR (production run) markers (e.g., CAR, Crx, Recoverin and Rhodopsin), a crucial monitoring of non-targeted cell populations was established (FIGS. 9, 11-12), for in process monitoring of product safety specifications. For example, Production run PR-46 testing at week 8 for the expression of undifferentiated cell markers TRA-1-60 and SSEA5 detected high levels (>1%, FIG. 11) of positive cells, and that production run was halted. In addition, cells at week 8 and week 15 of PR-44 and PR-45, were dissociated by TrypLE Select and seeded on LN521 coated TC flasks. These flattened cultures were harvested 2 weeks later, and aliquots were frozen for testing by flow cytometry. Significant difference is demonstrated between the control static cultures and the dynamic cultures, specifically showing increased expression of photoreceptors (PR) markers such as CRX and Recoverin and the end of 16 weeks differentiation in dynamic conditions versus the static control. Interestingly, shifting from the non-adherent dynamic culture to adherent static culture increased the expression of Cone Arrestin (CAR) almost 10 folds, increasing yet again the prospects of industrial grade process development from a product quality aspect.

### Example 4-Quantification of Cell Metabolism to Monitor Viable Cell Amount

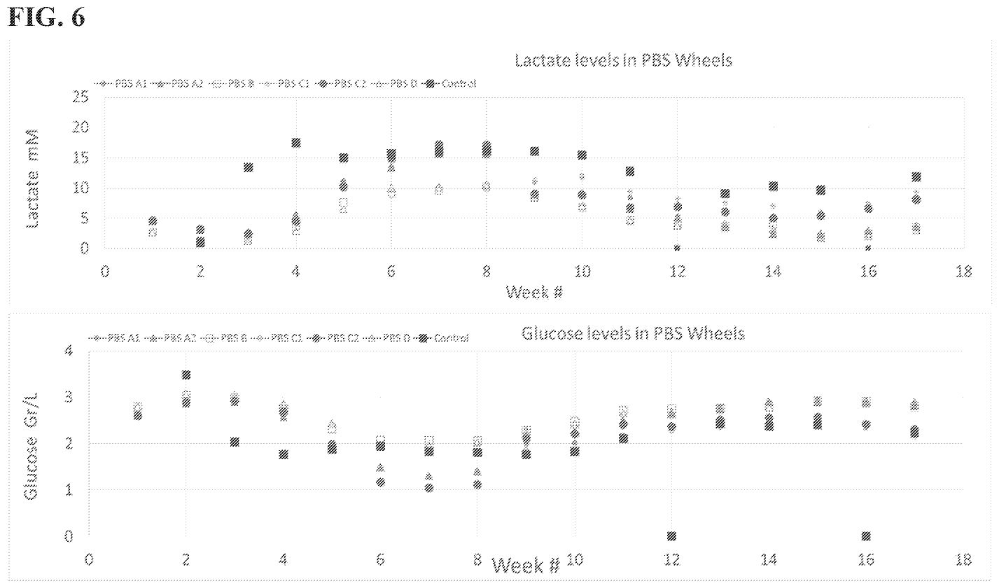

Glucose and lactate levels were determined before each media replacement. Metabolite levels correspond to the amount of living cells, where glucose levels reduce and lactate level increases as more living cells are present. The graphs shown in FIG. 6 demonstrate that glucose consumption of groups A and C of PR-44 was similar to the control static group throughout the study. Taking into account that Groups A and C of PR-44 were split at week 8, and more cells were sampled out of their vessels through differentiation, the difference in the initial seeded cells was maintained throughout the process. Increasing agitation velocity (Groups C1 and C2 of PR-44) also increased metabolism (i.e. lower glucose and higher lactate then lower velocities), while increasing IGF-1 concentration didn't seem to significantly affect metabolism (for example groups C1 Vs. C2 of PR-44).

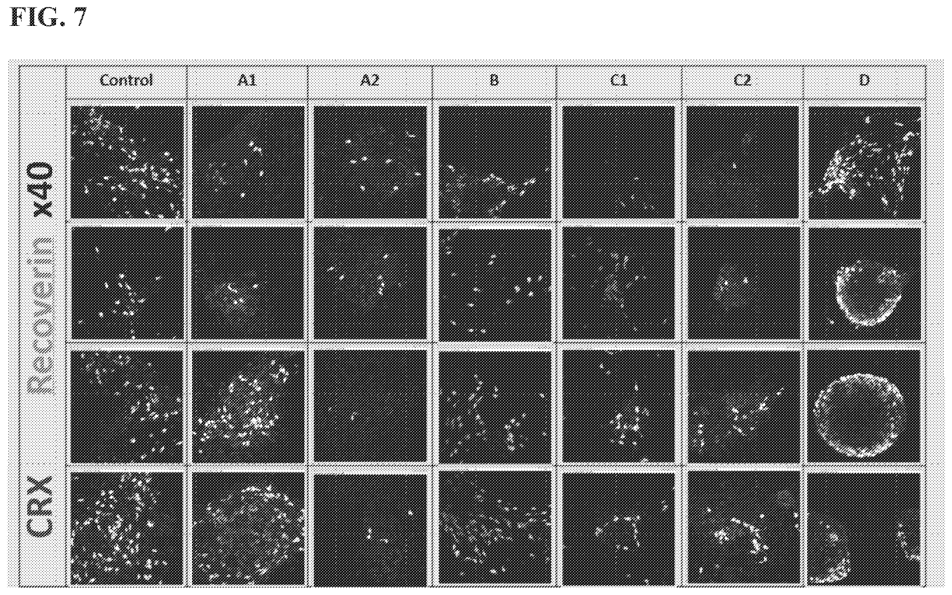

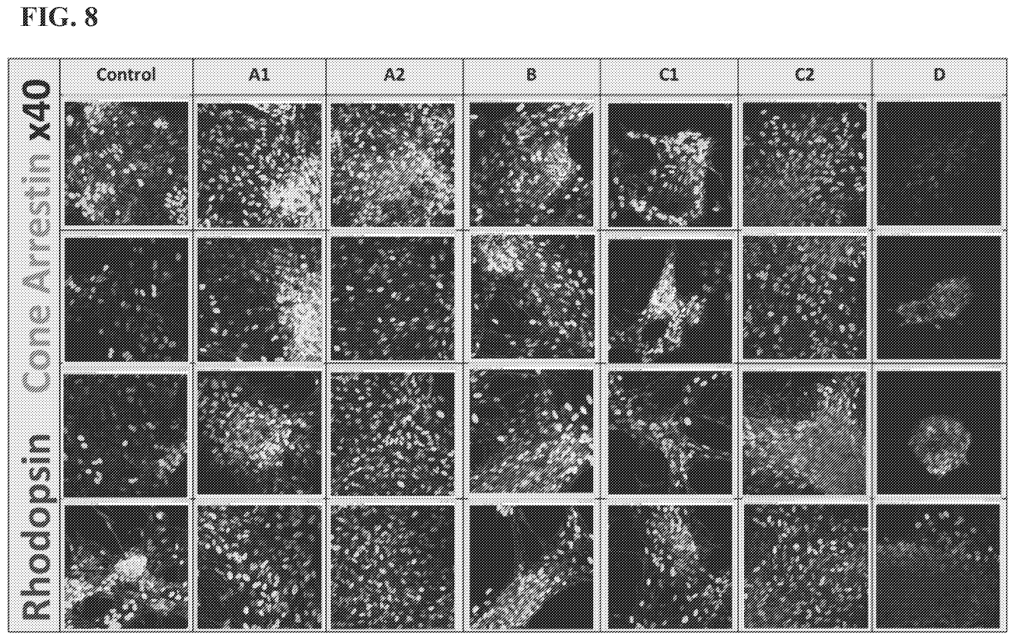

Cells were collected from the bioreactors after 8 or 16 weeks, dissociated and seeded for 2 weeks on PDL/laminin coated cover glasses. Immunostaining clearly identified the different PR cell populations, including the early photoreceptors. Double staining with CRX and Recoverin antibodies demonstrated a large population of cells expressing CRX and some cells expressing Recoverin (FIG. 7 and FIG. 9). Interestingly, Recoverin staining illuminated relatively long neurites in all conditions where IGF-1 was used at 5 ng/mL. In contrast, in the conditions where IGF-1 was used at 10 ng/mL, the positive cells had significantly shorter nerite, indicating that high IGF-1 may maintain cells at a relatively immature state. Immunostaining with both anti-CAR antibody and anti-Rhodopsin antibody identified two distinct mature photoreceptors populations: a large Cone Arrestin (CAR) positive cell population indicated (i.e. Cone photoreceptors), and the Rhodopsin positive cells (FIG. 8). The labeling of the Rhodopsin antibody was mainly at the cells periphery.

### REFERENCES

Transl Vis Sci Technol. 2019 May 30; 8 (3):30.
Reichman, S. et al., Generation of Storable Retinal Organoids and Retinal Pigmented Epithelium from Adherent Human iPS Cells in Xeno-Free and Feeder-Free Conditions Stem Cells 2017(5):1176-1188.
Gonzalez-Cordero, A. et al. Recapitulation of Human Retinal Development from Human Pluripotent Stem Cells Generates Transplantable Populations of Cone Photoreceptors. Stem Cell Reports 2017, 9:820-837.
EP3583204A1
EP3761998A1
EP2796545A1
Meyer, S. et al, Stem Cells 29 (2011) 1206-1218.
Eiraku, M. et al., Self-organizing optic-cup morphogenesis in three-dimensional culture, Nature 472 (2011) 51-56.
Meyer, J. et al., Modeling early retinal development with human embryonic and induced pluripotent stem cells, Proc. Natl. Acad. Sci. U.S.A 106(2009) 0.16703-16698.
Nakano, T. Self-formation of optic cups and storable stratified neural retina from human ESCs, Cell Stem Cell 10 (2012) 771-785.
Osakada, F. et al. Nat. Biotechnol. 26 (2008): 224-215.
Osakada, F. et al., Stepwise differentiation of pluripotent stem cells into retinal cells, Nat. Protoc. 4 (2009) 811-824.
Phillips, M. et al., Blood-derived human iPS cells generate optic vesicle-like structures with the capacity to form retinal laminae and develop synapses, Invest. Ophthalnol. Vis. Sci. 53 (2012) 2007-2019.
Reichman, S. et al., From confluent human iPS cells to self-forming neural retina and retinal pigmented epithelium, Proc. Natl. Acad. Sci. U.S.A 111 (2014) 8518-8523.
Zhong, X., et al. Generation of three-dimensional retinal tissue with functional photoreceptors from human iPSCs, Nat. Commun. 5 (2014) 4047.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All references, e.g., U.S. patents, U.S. patent application Publications, PCT patent applications designating the U.S., published foreign patents and patent applications cited herein are incorporated herein by reference in their entireties. GenBank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

While this invention has been particularly shown and described with references to preferred embodiments thereof, it will be understood by those skilled in the art that various changes in form and details may be made therein without departing from the scope of the invention encompassed by the appended claims.

What is claimed is:

1. A pharmaceutical composition comprising a population of retinal cells, wherein:
(a) greater than or equal to 10% of the cells in the population express Cone-rod homeobox (Crx);
(b) greater than or equal to 3% of the cells in the population express Recoverin;
(c) greater than or equal to 3% of the cells in the population express Cone arrestin (CAR); and
(d) less than or equal to 1% of the cells in the population express TRA-1-60 and/or SSEA5;
wherein the pharmaceutical composition comprises a pharmaceutically acceptable carrier.

2. The pharmaceutical composition of claim 1, wherein greater than or equal to 15% of the cells in the population express Crx.

3. The pharmaceutical composition of claim 1 or 2, wherein greater than or equal to 5% of the cells in the population express Recoverin.

4. The pharmaceutical composition of any one of claims 1-3, wherein greater than or equal to 6% of the cells in the population express CAR.

5. The pharmaceutical composition of any one claims 1-4, wherein the population of retinal cells comprises cells that express at least one marker selected from the group consisting of SIX homeobox 3 (six3), SIX homeobox 6 (six6), phosphodiesterase 6H (PDE 6H), visual system homeobox 2 (CHX10 or VSX2), premelanosome protein (PMEL), protein kinase C alpha (PKCa), ELAV like RNA binding protein 3/4 (HuC/D), orthodenticle homeobox 2 (Otx2), neuronal differentiation 1 (NeuroD), B lymphocyte-induced maturation protein-1 (Blimp1), Transducin, Phosducin (PdC), retinoid X receptor gamma (RXRv), thyroid hormone receptor isoform (Tr-32), atonal bHLH transcription factor 7 (Atoh7), insulin gene enhancer protein (Isl-1), retinal homeobox gene 1 (Rx1), paired box 6 (Pax6), LIM homeobox 2 (LHX2), and retina and anterior neural fold homeobox (RAX).

6. The pharmaceutical composition of claim 5, wherein:
(e) greater than or equal to 30% of the cells in the population express Pax6;
(f) less than or equal to 40% of the cells in the population express beta tubulin 3;
(g) less than or equal to 30% of the cells in the population express PMEL;
(h) less than or equal to 30% of the cells in the population express PKCa; or
(i) less than or equal to 10% of the cells in the population express HuCD.

7. The pharmaceutical composition of any one of claims 1-6, wherein less than or equal to 0.1% of the cells in the population express TRA-1-60 and/or SSEA5.

8. A pharmaceutical composition of any one of claims 1-7, wherein:
(a) between about 10% to 70% of the cells in the population express Crx;
(b) between about 3% to 90% of the cells in the population express Recoverin;
(c) between about 3% to 90% of the cells in the population express CAR; and
(d) between 0 to about 0.1% of the cells in the population express TRA-1-60 and/or SSEA5.

9. The pharmaceutical composition of any one of claims 1-8, wherein the population of retinal cells comprises early eye field cells, embryonic retinal cells, precursors of photoreceptor neuronal cells (PNCs), mature photoreceptor neuronal cells, or any combination thereof.

10. The pharmaceutical composition of claim 9, wherein:
(i) the early eye field cells comprise cells expressing Six3, Six 6, Rx1, Pax6, RXRγ, LHX2 and/or RAX;
(ii) the embryonic retinal cells comprise cells express Otx2, NeuroD, Blimp1, Transducin, Phosducin (PdC), RXRy and Tr-32, Atoh7 and/or Isl-1; or
(iii) the precursors of PNCS and mature PNCs comprise cells expressing Crx, Recoverin, and/or Cone arrestin (CAR).

11. The pharmaceutical composition of any one of claims 1-10, wherein the population of retinal cells comprises neuronal retina cells (NRCs), Muller glia cells, retinal pigmented epithelial (RPE) cells, or any combination thereof.

12. The pharmaceutical composition of claim 11, wherein the NRCs comprise PNCs, retinal ganglion cells, horizontal neurons, amacrine neurons, rod bipolar cells, cone bipolar cells, rod photoreceptor cells, cone photoreceptor cells or any combination thereof.

13. The pharmaceutical composition of any one of claims 1-12, comprising a cryopreservation medium.

14. The pharmaceutical composition of claim 13, wherein the cryopreservation medium comprises a cryoprotective agent selected from the group consisting of glycerol, sucrose, dextran and dimethyl sulfoxide (DMSO).

15. The pharmaceutical composition of claim 14, wherein the pharmaceutical composition comprises about 0.10% to about 40% of the cryoprotective agent.

16. The pharmaceutical composition of claim 14, wherein the pharmaceutical composition comprises about 1% to about 10% of the cryoprotective agent.

17. The pharmaceutical composition of any one of claims 1-16, wherein the population of retinal cells comprises between 5,000 cells and 25 million cells.

18. The pharmaceutical composition of any one of claims 1-17, wherein the population of retinal cells are at a concentration of 1×10⁵ cells per mL to about 100×10⁶ cells per mL.

19. The pharmaceutical composition of any one of claims 1-17, wherein the population of cells are in a suspension.

20. The pharmaceutical composition of any one of claims 1-17, wherein the population of cells are in a scaffold.

21. The pharmaceutical composition of claim 20, wherein the scaffold is biocompatible and/or biodegradable.

22. A method of making the pharmaceutical composition of any one of claims 1-21, comprising culturing a population of undifferentiated pluripotent stem cells under culture conditions sufficient to:
(i) differentiate cells in the population of the undifferentiated cells into retinal cells; and
(ii) produce a cellular aggregate, wherein the cellular aggregate is between 100 μm-800 μm in diameter.

23. A method of producing a composition comprising a population of retinal cells, the method comprising:
(a) culturing a population of undifferentiated pluripotent stem cells in a first cell culture medium comprising Nicotinamide (NIC), rel-4-[(3aR,4S,7R,7aS)-1,3,3a,4,7,7a-Hexahydro-1,3-dioxo-4,7-methano-2H-isoindol-2-yl]-N-8-quinolinylbenzamide (IWRe), and Rock inhibitor (RI) for a first time period;
(b) culturing the population of cells produced in step (a) in a second cell culture medium comprising NIC and IWRe, for a second time period;
(c) culturing the population of cells produced in step (b) in a third cell culture medium comprising NIC and Insulin-like growth factor 1 (IGF-1) for a third time period; and
(d) collecting the population of cells, thereby producing the composition comprising the population of retinal cells;
wherein the populations of cells in are cultured for at least 14 weeks in a culture vessel under conditions sufficient to produce cellular aggregates that are 100 μm to 800 μm in diameter.

24. The method of claim 23, wherein the cells are cultured in dynamic culture conditions.

25. The method claim 23 or 24, wherein the culture vessel is a bioreactor.

26. The method of any one of claims 23-25, wherein the culture vessel comprises a vertical wheel bioreactor, a wave bioreactor, or a Gas Permeable Rapid Expansion bioreactor.

27. The method of claim 26, wherein the culture vessel comprises a wheel bioreactor, and wherein rotational velocity of the wheel bioreactor is increased at each of steps (a)-(c) thereby producing cellular aggregates that are 100 μm to 800 μm in diameter that are suspended in the wheel bioreactor.

28. The method of claim 27, wherein the rotational velocity of the wheel bioreactor subjects the cellular aggregates to controlled shear stress.

29. The method of any one of claims 26-28, wherein the rotational velocity is between 35 to 80 revolutions per minute (RPM).

30. The method of any one of claims 26-29, comprising an initial rotational velocity of about 30-50 RPM that is increased to about 60-80 RPM by step (e).

31. The method of the any one of claims 23-30, wherein:
(i) the first time period is between 1-15 days,
(ii) the second time period is between 1-30 days, and/or
(iii) the third time period is between 5 days and 14 weeks.

32. The method of any one of claims 23-31, comprising, before step (d), a step comprising (i) culturing the population of cells produced in step (c) in a fourth cell culture medium comprising IGF-1, NIC, and tert-Butyl (S)-{(2S)-2-[2-(3,5-difluorophenyl)acetamido]propanamido}phenylacetate (DAPT) for a fourth time period.

33. The method of claim 32, wherein the fourth time period is between 1 week to 18 weeks.

34. The method of claim 32 or 33, comprising, after step (i), a step (ii) comprising culturing the population of cells in step (i) in a fifth cell culture medium comprising IGF-1, Retinoic acid (RA), Taurine (TA), Brain-derived neurotropic factor (BDNF), and Neurorophin-4 (NT4) for a fifth time period.

35. The method of claim 34, wherein the fifth time period is 4 weeks to 24 weeks.

36. The method of any one of claims 23-35, wherein the RI is at a concentration of between about 1-20 μM in the first cell culture medium.

37. The method of any one of claims 23-36, wherein the NIC is at a concentration of between about 1-50 mM in the first, second, third, and/or fourth cell culture medium.

38. The method of any one of claims 23-37, wherein the IWRe is at a concentration of between about 0.01-20 μM in the first and/or second cell culture medium.

39. The method of any one of claims 23-38, wherein the IGF-1 is at a concentration of between about 0.5-20 ng/mL in the third, fourth, and/or fifth cell culture medium.

40. The method of any one of claims 32-39, wherein the DAPT is at a concentration of between about 1-50 μM in the fourth cell culture medium.

41. The method of any one of claims 34-40, wherein the BDNF is at a concentration of between about 5-50 ng/mL in the fifth cell culture medium.

42. The method of any one of claims 34-41, wherein the NT4 is at a concentration of between about 2-200 ng/mL in the fifth cell culture medium.

43. The method of any one of claims 34-42, wherein the TA is at a concentration of between 10-400 μM in the fifth cell culture medium.

44. The method of any one of claims 23-43, wherein the undifferentiated pluripotent stem cells comprise human embryonic stem cells (hESCs) or human induced pluripotent stem cells (hiPSCs).

45. The method of claim 44, wherein the hESCs comprise HADC102 cells.

46. The method of any one of claims 23-45, wherein the population of retinal cells comprises early eye field cells, embryonic retinal cells, precursors of photoreceptor neuronal cells (PNCs), mature photoreceptor neuronal cells, or any combination thereof.

47. The method of any one of claims 23-46, wherein the population of retinal cells comprises neuronal retina cells (NRCs), Muller glia cells, retinal pigmented epithelial (RPE) cells, or any combination thereof.

48. The method of claim 47, wherein the NRCs comprise PNCs, retinal ganglion cells, horizontal neurons, amacrine neurons, rod bipolar cells, cone bipolar cells, rod photoreceptor cells, cone photoreceptor cells or any combination thereof.

49. The method of any one of claims 23-48, comprising cryopreserving the population of retinal cells.

50. The method of claim 49, wherein the cryopreservation comprises suspending the population of cells in a cryopreservation medium to form a cell suspension and storing the cell suspension at less than or equal to −80° C., or less than or equal to −140° C.

51. The method of claim 50, wherein the cryopreservation comprises a cryopreservation medium suitable for administration to the eye of a subject.

52. The method of claim 50 or 51, wherein the cryopreservation medium comprises a cryoprotective agent selected from the group consisting of glycerol, sucrose, dextran, and dimethyl sulfoxide (DMSO).

53. The method of claim 52, wherein the cryopreservation medium comprises about 0.1% to about 40% of a cryoprotective agent.

54. The method of claim 52, wherein the pharmaceutical composition comprises about 1% to about 10% of the cryoprotective agent.

55. The method of claim 50 or 51, wherein the cryopreservation medium comprises 2% DMSO, 5% DMSO, or 10% DMSO.

56. The method of any one of claims 50-55, wherein the cryopreservation medium comprises adenosine, dextran-40, lactobionic acid, HEPES (N-(2-Hydroxyethyl) piperazine-N′-(2-ethanesulfonic acid)), sodium hydroxide, L-glutathione, potassium chloride, potassium bicarbonate, potassium phosphate, dextrose, sucrose, mannitol, calcium chloride, magnesium chloride, potassium hydroxide, sodium hydroxide, water, or a combination thereof.

57. The method of any one of claims 23-56, comprising, prior to step (a), seeding the undifferentiated pluripotent stem cells as single cells in a culture vessel at a density of 50,000 cell/mL to about 2,000,000 cells/mL.

58. The method of any one of claims 23-57, comprising, prior to step (d):
(i) collecting the cellular aggregates;
(ii) dissociating the cellular aggregates to produce dissociated cells;
(iii) seeding the dissociated cells in tissue culture flasks; and
(iv) culturing the dissociated cells under adherent static conditions for at least one week.

59. The method of claim 58, wherein the dissociated cells are cultured for 1-3 weeks.

60. A method of producing a composition comprising a population of retinal cells, the method comprising:
(a) culturing a population of undifferentiated pluripotent stem cells in a first cell culture medium comprising Nicotinamide (NIC) at a concentration of 10 mM, rel-4-[(3aR,4S,7R,7aS)-1,3,3a,4,7,7a-Hexahydro-1,3-dioxo-4,7-methano-2H-isoindol-2-yl]-N-8-quinolinylbenzamide (IWRe) at a concentration of 3 μm, and Rock inhibitor (RI) at a concentration of 10 μm for at least one day;
(b) culturing the population of cells produced in step (a) in a second cell culture medium comprising NIC at a concentration of 10 mM, and IWRe at a concentration of 3 μm for at least 2 days;
(c) culturing the population of cells produced in step (b) in a third cell culture medium comprising NIC at a concentration of 10 mM and IGF-1 at a concentration of 5 ng/mL for at least 10 days; and
(d) collecting the population of cells, thereby producing the composition comprising the population of retinal cells;
wherein the populations of cells in are cultured for at least 14 weeks in a culture vessel under conditions sufficient to produce aggregates that are 100 μm to 800 μm in diameter.

61. A method of producing a composition comprising a population of retinal cells, the method comprising:
(a) culturing a population of undifferentiated pluripotent stem cells in a first cell culture medium comprising Nicotinamide (NIC) at a concentration of 10 mM, rel-4-[(3aR,4S,7R,7aS)-1,3,3a,4,7,7a-Hexahydro-1,3-dioxo-4,7-methano-2H-isoindol-2-yl]-N-8-quinolinylbenzamide (IWRe) at a concentration of 3 μm, and Rock inhibitor (RI) at a concentration of 10 μm for at least one day;
(b) culturing the population of cells produced in step (a) in a second cell culture medium comprising NIC at a concentration of 10 mM and IWRe at a concentration of 3 μm, for at least 2 days;
(c) culturing the population of cells produced in step (b) in a third cell culture medium comprising NIC at a concentration of 10 mM and IGF-1 at a concentration of 5 ng/mL for at least 10 days; and
(d) culturing the population of cells produced in step (c) in a fourth cell culture medium comprising IGF-1 at a concentration of 5 ng/mL, NIC at a concentration of 10 mM, and tert-Butyl (S)-{(2S)-2-[2-(3,5 difluorophenyl)acetamido]propanamido}phenylacetate (DAPT) at a concentration of 10 M for at least 2 weeks; and
(e) collecting the population of cells, thereby producing the composition comprising the population of retinal cells;
wherein the populations of cells in are cultured for at least 14 weeks in a culture vessel under conditions sufficient to produce aggregates that are 100 μm to 800 μm in diameter.

62. A method of producing a composition comprising a population of retinal cells, the method comprising:
(a) culturing a population of undifferentiated pluripotent stem cells in a first cell culture medium comprising Nicotinamide (NIC) at a concentration of 10 mM, rel-4-[(3aR,4S,7R,7aS)-1,3,3a,4,7,7a-Hexahydro-1,3-dioxo-4,7-methano-2H-isoindol-2-vl]-N-8-quinolinylbenzamide (IWRe) at a concentration of 3 μm, and Rock inhibitor (RI) at a concentration of 10 μm for at last 1 day;
(b) culturing the population of cells produced in step (a) in a second cell culture medium comprising NIC at a concentration of 10 mM and IWRe at a concentration of 3 μm for at least 2 days;
(c) culturing the population of cells produced in step (b) in a third cell culture medium comprising NIC at a concentration of 10 mM and IGF-1 at a concentration of 5 ng/mL for at least 10 days;
(d) culturing the population of cells produced in step (c) in a fourth cell culture medium comprising IGF-1 at a concentration of 5 ng/mL, NIC at a concentration of 10 mM, and tert-Butyl (S)-{(2S)-2-[2-(3,5 difluorophenyl)acetamido]propanamido}phenylacetate (DAPT) at a concentration of 10 μM for at least 2 weeks;
(e) culturing the population of cells produced in step (d) in a fifth cell culture medium comprising IGF-1 at a concentration of 5 ng/mL, Retinoic acid (RA) at a concentration of 0.5 μM, Taurine (TA) at a concentration of 100 μM, Brain-derived neurotropic factor (BDNF) at a concentration of 20 ng/mL, and Neurorophin-4 (NT4) at a concentration of 20 ng/mL for at least 5 weeks; and
(f) collecting the population of cells, thereby producing the composition comprising the population of retinal cells;
wherein the populations of cells in are cultured for at least 14 weeks in a culture vessel under conditions sufficient to produce aggregates that are 100 μm to 800 μm in diameter.

63. The method of any one of claims 60-62, wherein the culture vessel comprises a wheel bioreactor, and wherein rotational velocity of the wheel bioreactor is increased at each of steps (a)-(c) thereby producing cellular aggregates that are 100 μm to 800 μm in diameter that are suspended in the wheel bioreactor.

64. A pharmaceutical composition, produced by the methods of any one of claims 22-63.

65. A method of treating a subject with a vision condition, comprising administering a therapeutically effective amount of the pharmaceutical composition of any one of claims 1-21 or 64 to an eye of the subject.

66. A method treating a subject with a vision condition, comprising administering a therapeutically effective amount of a pharmaceutical composition comprising a population of retinal cells, wherein:
(a) between about 10% to 70% of the cells in the population express Crx;
(b) between about 3% to 90% of the cells in the population express Recoverin;
(c) between about 3% to 90% of the cells in the population express CAR; and
(d) between 0 to about 0.1% of the cells in the population express TRA-1-60 and/or SSEA5;
wherein the composition is administered eye of the subject.

67. The method of claim 65 or 66, wherein the composition is administered via injection to the retina.

68. The method of claim 67, wherein the injection comprises intravitreal, subretinal or suprachoroidal injection of a suspension comprising the population of cells.

69. The method of claim 65 or 66, comprising implantation of a scaffold comprising the population of cells.

70. The method of claim 69, wherein the scaffold is implanted into the subretinal space.

71. The method of any one of claims 65-70, wherein the vision condition comprises a neurodegenerative disease of the retina or damage to the retina.

72. The method of claim 71, wherein the neurodegenerative disease of the retina comprises Stargardt's disease, diabetic retinopathy, macular degeneration, retinitis pigmentosa, Leber congenital amaurosis, cone-rod dystrophym, choroideremia, or X-linked retinoschisis.

73. The method of claim 72, wherein the macular degeneration comprises age related macular degeneration.
